(19) United States
(12) Patent Application Publication
(10) Pub. No.: US 2022/0408730 A1
(43) Pub. Date: Dec. 29, 2022

(54) PLANT TREATMENT

(71) Applicant: INSTITUTO SUPERIOR DE AGRONOMIA, Lisboa (PT)

(72) Inventors: Giovanni DEL FRARI, Lestans (IT); Maria Helena MENDES DA COSTA FERREIRA CORREIA DE, Vila Franca de Xira (PT); Ana Isabel GUSMÃO LIMA, Amora (PT); Ricardo Manuel DE SEIXAS BOAVIDA FERREIRA, Lisboa (PT)

(73) Assignee: INSTITUTO SUPERIOR DE AGRONOMIA, Lisboa (PT)

(21) Appl. No.: 17/616,918

(22) PCT Filed: Jun. 5, 2020
(86) PCT No.: PCT/EP2020/065742

§ 371 Date: Dec. 6, 2021

(30) Foreign Application Priority Data
Jun. 6, 2019 (PT) 115569

(51) Int. Cl. A01N 63/30; A01N 63/20; C07K 14/415; A01N 65/08

(52) U.S. Cl. CPC A01N 63/30; A01N 63/20; C07K 14/415; A01N 65/08; C12R 2001/645

(57) ABSTRACT

Use of an adaptogen for the treatment and/or control and/or prevention of a disease or infestation of a plant by application of said adaptogen to the plant, wherein:
(i) the adaptogen is Blad or a variant thereof and the location of the disease or infestation is in a region of the plant which is different to the region where the adaptogen is applied; or
(ii) the adaptogen is skopobiota which is used to inoculate said plant or part thereof;
   and preferably said adaptogen is in the form of a composition.

### FIELD OF THE INVENTION

The invention relates to treating a plant.

### BACKGROUND TO THE INVENTION

The dynamics of our civilization is leading us to a situation where ultimately all pathogenic microorganisms will be present at all locations. Human mobility and activity, human-derived climate change and our struggle against human, animal and plant pathogens and pests are at the forefront of this state of affairs. To feed the over 7 billion world population we have no alternative but to use toxic (phyto)pharmaceutical products. As a result, we are inevitably pushing the evolution of undesirable organisms and complex macromolecular structures (ranging from weeds and insects, through fungi and bacteria, to virus) towards areas which are becoming increasingly difficult to address by researchers.

### SUMMARY OF THE INVENTION

The inventors work relates to the susceptibility of plants to disease and infestation, as well as molecules and mechanisms that can be used to protect plants from disease and infestation. The invention relates to uses and methods relating to treating plants, as well as to protected plants and specific compositions containing two or more microorganisms that can be used to protect plants or in the case of a synergistic application of BLAD as described herein, the compositions may contain one or more microorganism of the kind in question. In preferred embodiments, the invention comprises specific compositions containing two or more microorganisms that can be used to protect plants. The invention also relates to identifying plants which are in need of treatments, and optionally treating them, for example as described herein.

The invention provides use of the Blad protein or a variant thereof and/or use of skopobiota for the treatment and/or control and/or prevention of a disease or infestation of a plant, wherein:
said Blad or said variant is applied to the plant, wherein the location of the disease or infestation is in a region of the plant which is different to the region where the composition is applied; and/or
said skopobiota is applied to the plant or to a part thereof, typically as an inoculation.

The invention provides use of a composition for the treatment and/or control and/or prevention of a disease or infestation of a plant by application of said composition to the plant, wherein the location of the disease or infestation is in a region of the plant which is different to the region where the composition is applied, wherein said composition comprises a polypeptide comprising the Blad sequence shown in SEQ ID NO: 4 or an active variant thereof which has Blad activity and which comprises a sequence which has at least 70% identity to either SEQ ID NO: 4 or a fragment of SEQ ID NO: 4 which is at least 100 amino acids in length.

The invention also provides use of a composition comprising skopobiota for the treatment and/or control and/or prevention of a disease or infestation of a plant by inoculation of said plant or part thereof with said composition.

The invention provides combined use of Blad or a variant thereof with skopobiota. The invention provides a method of treatment and/or control and/or prevention of a disease or infestation of a plant by inoculation of said plant or part thereof with a composition containing skopobiota and by the external application of a composition containing an antimicrobial agent comprising a polypeptide comprising the Blad sequence shown in SEQ ID NO: 4 or an active variant thereof which has antimicrobial activity and which comprises a sequence which has at least 70% identity to either SEQ ID NO:4 or a fragment of SEQ ID NO: 4 which is at least 100 amino acids in length.

The invention provides a method of detection of a disease in the vasculature of an externally asymptomatic plant comprising the steps of inoculation of said plant or part thereof with a composition containing skopobiota, the external application of a composition containing an antimicrobial agent comprises a polypeptide comprising the Blad sequence shown in SEQ ID NO:4 or an active variant thereof which has antimicrobial activity and which comprises a sequence which has at least 70% identity to either SEQ ID NO:4 or a fragment of SEQ ID NO:4 which is at least 100 amino acids in length; and obtaining an image of part of the vasculature of said plant by X-ray or tomography in order to determine the extent of any infection present therein.

The invention provides a method of assessing the health of a plant comprising typing the microbiome of the plant to thereby determine whether the plant is in need of inoculation with microorganisms to improve its microbiome, wherein the typing preferably comprises DNA barcoding of the microbiome.

The invention provides a typing method which can be used to select a plant for treatment according to any use or method as described herein.

The invention provides a plant which is obtained by any use or method of the invention which relates to treatment of a plant, which optionally relates to applying Blad or a variant thereof or a skopobiota. The invention provides a skopobiota as described herein, or which is suitable for use in any use or method of the invention.

### BRIEF DESCRIPTION OF THE FIGURES

FIG. 1 shows 0-conglutin precursor, 531-amino acid residue sequence (61,931.39 Da). The Blad 173-amino acid residue sequence (20,408.95 Da) is highlighted in yellow (Monteiro et al, 2003, 2006).

FIG. 2. Barplots of the relative abundances of the 20 most abundant taxa identified to species (s) or genus (g) level, found in rooted grapevine cuttings non-inoculated (Water), or inoculated with Phaeomoniella chlamyobspora (Pathogen) or a consortium of wood endophytes (Skopobiota) or a combination of both (Pathogen+Skopobiota). Grapevines were treated with either Blad-containing oligomer (BCO) or potassium permanganate (Control) or copper oxychloride and sulfur (CuS) or fosetyl-aluminium and penconazol (Systemics). ‘Unassigned’ are taxa identified to a lower taxonomic level than family or non-identified, ‘Others’ are taxa not included in the 20 most abundant. On top, barplots grouped by ‘Inoculation’ type; bottom, barplots grouped by ‘Fungicide’ treatment.

FIG. 3 show two photos of a nontreated pruning wound and a treated pruning wound. Immediately after pruning, 50 μL of water (Left, control) or 50 μL of a Blad/BCO solution (right) were applied onto the wounds. The photos were taken at the same plant a couple of months later, at bud break. Compare with the natural development state of the vineyard in the background. Twenty-four replicates were prepared for each treatment.

FIG. 4. Differential heat tree matrix depicting the changes in taxa abundance between different fungicide treatments. The size of the individual nodes in the grey cladogram depicts the number of taxa identified at that taxonomic level. The smaller cladograms show pairwise comparisons between each treatment, with the colour illustrating the log₂ fold change: a red node indicates a lower abundance of the taxon in the tissue group stated on the abscissa, than in the tissue group stated on the ordinate. A blue node indicates the opposite.

FIG. 5 shows ecological networks of interaction among taxa, under different fungicide treatments. Individual taxa are represented by nodes and interactions are represented by undirected lines.

FIG. 6 shows the L. albus β-conglutin precursor encoding sequence (SEQ ID NO: 1).

FIG. 7 shows the internal fragment of the β-conglutin precursor encoding sequence that corresponds to Blad (SEQ ID NO: 3).

FIG. 11 shows Blad's predicted secondary structure.

FIG. 12 compares the amino acid numbers of variants.

FIG. 13 relates to Blad-153.

FIG. 14 relates to Blad-169.

FIG. 15 relates to Blad-173.

FIG. 16 relates to Blad-185.

FIG. 17 compares variants of Blad.

FIG. 18 compares Blad-173 and variants.

FIG. 19 relates to Blad-127.

FIG. 20 relates to Blad-147.

FIG. 21 relates to Blad-199.

FIG. 22 relates to Blad experiment number 1.

FIG. 23 relates to Blad experiment number 2.

FIG. 24 compares Blads-127, -147 and -199.

FIG. 25 relates to Blad experiment number 3.

FIG. 26 compares the biological activity of Blads-127, -147 and -199 with Blad-173.

### DETAILED DESCRIPTION

Overall Summary

This application describes three approaches which may be applied individually or in combination to address/target, specifically and efficiently, in both preventive and curative ways, plants diseases in general and, specifically, those recalcitrant diseases currently under uncontrolled dissemination and for which neither control nor treatment exist because their causal agents thrive in plant locations which are inaccessible to our chemical treatments (e.g. inside the dead xylem of woody tissues) or in the soil. The three approaches aim at maintaining (i.e. not disturbing) and/or improving/enhancing the stability of the plant/soil microbiotas in a balanced way, rather than killing the pathogens.

A healthy plant comprises several microbiotas, some of which live within each organ (from the seed to the dead xylem in the case of woody plants), whereas others live in close contact with the plant surface (e.g. leaves and roots). An increasing body of evidence suggests that many plant diseases result from imbalances in the healthy microbiota in a way quite similar with the human gut microbiota when we ingest, for example, a broad-spectrum antibiotic.

Several causes are responsible for the imbalances that occur in plant microbiotas, most notably intensive pesticide applications or the ‘blind’ application of huge numbers of one or a few microbial species, as biological pesticides, which may reduce the prevalence of a specific pathogen but further disrupt the already unstable microbiota imbalance. Thus, for example, an intensively cultivated vineyard must often be subjected to 10 to 14-day interval fungicide applications to minimize the risk of losing most or all grape production due to the downy and powdery mildews. However, if that very same vineyard is abandoned and left on its own, grapes are produced and the plants seem to cope and live with the causal agents of those diseases.

A number of plant diseases are spreading rapidly across the globe, for which we have neither ways of treatment nor methods to control their dissemination. Even if we did, the generalized application of unspecific fungicides kills the majority of the mycobiota, creating favorable conditions for the free-from-competition very fast grow of the first fungal species that will ensue. The most notorious examples are the microbial pathogens that grow in the soil and infect/enter the plants via the roots and those which thrive in the woody tissues of perennial plants. Well known examples include:
the oomycota Phytophthora cinamommi (a soilborne root- and wood-infecting pathogen) in cork oak, chestnut trees and a huge number of other plants,
the oomycote belonging to the genus Pythium, Phytophthora (e.g. P. infestans), and fungi belonging to the genus Fusarium (soilborne root- and aerial part-infecting pathogens) in several horticultural species (carrots, tomato and potato),
the protozoan Plasmodiophora brassicae (a soilborne root-infecting pathogen) in several Brassica,
the fungal cluster (wood-infecting pathogens) responsible for grapevine trunk diseases, and
the bacterium Xylella fastidiosa (a wood-infecting pathogen) in grapevine and olive trees.

The present application addresses methodologies which aim at modulating, without killing, the microbiota either in a preventive or curative way. Depending on the location of the target microbiota, three different tools may be combined and used to maintain/modulate it in a local or distant organ (including the rhizosphere) in a balanced way:

(i) A prebiotic, such as Blad (a component of a large oligomer untranslocatable within the plant, and a known contact fungicide and plant biostimulant), which when applied on a plant tissue (e.g. sprayed onto the leaves) will modulate the microbiota in another, often inaccessible, plant organ and/or the rhizosphere. In this way, Blad addition to a plant (e.g. sprayed onto the leaves) will prevent/treat several plant diseases without killing fungi and oomycetes but rather by balancing the microbiotas, either locally or in a faraway organ, including inaccessible tissues (e.g. the dead xylem of woody trunks and roots) and the rhizosphere.

(ii) A probiotic, such as a skopobiota, namely a specifically designed, exact and reproducible, mixture of fungi applied to the plant tissues (e.g. injected into the wood, sprayed onto leaves) or the soil with a given purpose, in the present case to maintain/improve/enhance the stability of microbiotas in an balanced way.

(iii) A means to deliver the skopobiota to the soil such as, for example, biochar, bentochar (a novel mixture composed of bentonite and biochar) or any other suitable porous material previously inoculated with the skopobiota which, upon addition to the soil, will deliver its load of microbes to the soil during an extended period of time.

(iv) The plant biostimulant effect that results from soil application of biochar, bentochar (a novel mixture composed of bentonite and biochar) or any other suitable porous material previously inoculated or not with a skopobiota which, upon addition to the soil, will deliver its load of microbes to the soil during an extended period of time.

(v) Prevention/treatment of several plant diseases without killing fungi but rather by balancing the microbiotas, either locally or in a faraway organ, including inaccessible tissues (e.g. the dead xylem of woody trunks and roots) and the rhizosphere, by soil application of biochar, bentochar (a novel mixture composed of bentonite and biochar) or any other suitable porous material previously inoculated or not with a skopobiota which, upon addition to the soil, will deliver its load of microbes to the soil during an extended period of time.

(vi) The synergistic effects that result from any combination of the above tools.

Particular Aspects

The invention has different aspects, including:
plant xylem (trunk, stem and root) disease or infestation control,
treating a plant using Blad or a variant of Blad,
changing the microbiome or skopobiota of a plant, for example for disease or infestation control.

As used herein the term ‘pathogen’ refers to both disease and infestation situations, for example caused by microorganisms, multicellular microorganisms and insects. The term ‘disease’ as used herein includes infestations, for example as caused by insects. As used herein the term ‘Blad’ covers use of its variants, including homologues of Blad, fragments of Blad or homologues of fragments of Blad. The invention is described herein with reference to its two main aspects, use of Blad and use of a skopobiota, either used alone or in combination. It is understood that all features described herein can be used with either of these two aspects, for example in terms of species of plant, type of plant, the status of the plant, the pathogen which is relevant.

The invention provides adaptogens for plants, including Blad as prebiotic-like compound and skopobiota as a probiotic. The invention relates to the plant mycobiome, but is not restricted to the mycobiome. The term ‘adoptogen’ as used herein includes both Blad and skopobiota.

The invention relates to the following as listed below. The adoptogens of the invention may be used in all these different aspects:
typing and altering the wood microbiome
use of microbes (and adaptogens) as antagonists
techniques used to study microbiomes, including growth on artificial culture media, metagenomics and metabarcoding
plant defense boosters
metabarcoding analysis and mycobiome exchange
metabarcoding analyses of woody tissues
microbiome strengthening, for example for plant health promotion.

Delivery of Skopbiota Using Porous Material

The invention provides uses and methods in which the skopobiota is delivered to the plant using a porous material. In such uses and methods the porous material is preferably applied to the soil in which the plant is growing. The uses and methods are preferably combined with any other use or method mentioned herein, for example those that relate to use of Blad. The invention provides a method of applying a skopobiota to a plant by application of said skopobiota to soil where the plant is growing, wherein a porous material, preferably comprising biochar or a mixture of bentonite and biochar, is contacted with the skopbiota to enhance delivery of the skopobiota to the plant. The skopobiota and porous material may be applied separately to the soil or in a combined form. In one embodiment they are both applied within 10 metres of the plant, for example within 5, 2 or 1 metre of the plant. The porous material is preferably in the form of spheres, for example with a volume of 0.5 to 8 mm³. The skopbiota is typically present in the final combination with the porous material at 1×10⁵ microbes per mL or 10⁵ conidia/mL. The invention includes a means to deliver skopobiota to the soil such as, for example, biochar, bentochar (a novel mixture composed of bentonite and biochar) or any other suitable porous material previously inoculated with the skopobiota which, upon addition to the soil, will deliver its load of skopobiota to the soil during an extended period of time. The plant biostimulant effect preferably results from soil application of biochar, bentochar (a novel mixture composed of bentonite and biochar) or any other suitable porous material previously inoculated or not with a skopobiota which, upon addition to the soil, delivers the skopobiota to the soil during an extended period of time. The porous material preferably comprises bentochar, typically made from bentonite and biochar. The bentochar may be produced by mixing volumes of bentonite, biochar dust and water, for example in a ratio of (1:2.6:1). In some embodiments the bentonite and biochar are present at a ratio range of 1:1.5 to 1:5. The bentochar may be made into spheres of different volumes (<8 mm³), which are preferably dried in the oven (50 C) overnight.

The porous material may be used with any skopobiota described herein, including any combination of organisms described herein. The porous material may be used with skopobiota comprising at least one or all of: Cladosporium, Penicillium, Trichoderma harzianum and Alternaria infectoria. The porous material may be used with skopobiota comprising at least one or all of: Alternaria, Aureobasidium, Didymella glomerata and Setophaeosphaeria citri.

In the method or use of the invention the skopobiota loaded porous material may typically be used at 1% to 20% v/v of soil, for example at 2% to 15% v/v with soil or 4% to 8% v/v with soil.

### Additional Aspects of the Invention

1. Blad is an adaptogen or adaptogenic substance of the prebiotic-like type with a mode of action which may therefore improve all plant ailments (diseases, plagues and abiotic stressors).
2. Application of the contact and non-translocable fungicide Blad at a site (e.g. leaves) to treat/control plant infections which are taking place far away from the application point (e.g. stem/trunk or roots, typically inaccessible from a pesticide point of view). The prior art understanding of Blad's fungicidal activity would indicate that in these circumstance it would not be effective nevertheless it would appear that according to this new application Blad would appear to possess ‘tele’-fungicide activity which constitutes a complete departure from the understanding in this field to date.
3. Application of Blad according to aspect 1 but in a preventive way.
4. Foliar application of Blad to treat/control esca-related infections in grapevine.
5. Foliar application of Blad to prevent esca-related infections in grapevine.
6. Foliar application of Blad to treat/control grapevine trunk diseases.
7. Foliar application of Blad prevent grapevine trunk diseases.
8. Foliar application of Blad to treat/control Phytophthora infections in herbaceous plants.
9. Foliar application of Blad to prevent Phytophthora infections in herbaceous plants.
10. Foliar application of Blad to treat/control Phytophthora infections in trees.
11. Foliar application of Blad to prevent Phytophthora infections in trees.
12. Foliar application of Blad to treat/control Phytophthora infections in any one of cork oak, Kiwi and chestnut trees.
13. Foliar application of Blad to prevent Phytophthora infections in any one of cork oak, Kiwi and chestnut trees.
14. Said infections include any of the infections described herein including Xylella fastidiosa.
15. A skopobiota is an adaptogen or adaptogenic group of microorganisms of the probiotic type with a mode of action which may therefore improve all plant ailments (diseases, plagues and abiotic stressors).
16. Inoculation with a skopobiota (a careful selection of several to many endophytes) with the objectives specified in aspects 1 to 13.
17. Inoculation with a skopobiota to control/treat any plant infection.
18. The application to the same plant of both Blad and a skopobiota exhibit complementary and synergistic bioactivities in what concerns all plant ailments (diseases, plagues and abiotic stressors).
19. Inoculation with a skopobiota to prevent any plant infection.
20. Inoculation with a skopobiota plus application of Blad with the objectives specified in aspects 1 to 13, to exploit the synergy between Blad application and skopobiota inoculation.
21. Inoculation with a skopobiota plus application of Blad to control/treat any plant infection.
22. Inoculation with a skopobiota plus application of Blad to prevent any plant infection.
23. Non-destructive detection of functional or non-functional xylem infection in nurseries by X-ray simple or tomographic methods together with any combination Blad application and/or skopobiota inoculation with the objectives specified in aspects 1 to 19.
24. Non-destructive detection of functional or non-functional xylem infection under field or greenhouse conditions by X-ray simple or tomographic methods together with any combination Blad application and/or skopobiota inoculation with the objectives specified in aspects 1 to 19.

Preferred Aspects

The use of Blad/BCO, a previously known contact (i.e. non-systemic) and, due to its chemical nature and large size, untranslocable fungicide, plant biostimulant and bacteriostatic agent against Gram—bacteria in the present submission focuses on its application at a site (e.g. leaves) to control pathogens which are located far away from the site of delivery/administration (e.g. in the trunk or roots, typically inaccessible even from a translocable and systemic pesticide point of view).

The mechanisms involved indicate a mode of action in the direction of modulating the microbiome located elsewhere from the point of application, hence strengthening of the holobiont's health. This operates as a ‘tele-antimicrobial activity’ or a ‘tele-microbiome modulating activity’, microbiome fortifier, since Blad/BCO interferes with the microbiome composition and is capable of controlling pathogens located far away (e.g. in the stem/trunk and roots, typically inaccessible from a pesticide point of view) from the application site (e.g. the leaves). Blad is therefore acting as an “adaptogen” for plants, including acting as a prebiotic-like agent, which interferes indirectly with the plant microbiome and that induces resistance to stresses and promotes overall plant health and growth. In this way, Blad/BCO may be favorably applied to all plant ailments (diseases, plagues and abiotic stressors). Promotion of plant growth and protection by direct contact with pathogenic fungi and Gram—bacteria are not novel approaches.

The skopobiota involves a careful selection of fungi that belong to the endosphere of the (woody or not) plant of interest, i.e. by inoculation the infected plant with a consortium of endophytes, involving those that are normally predominant in the healthy tissues under consideration. This is a novel approach, meaning that it may be applied to all plant ailments (diseases, plagues and abiotic stressors).

Blad will act as fortifier of the natural mycobiome, e.g. by making it ‘stronger’/more resistant to stressors in general (and fight pathogens, possibly several pathogens simultaneously, in particular). On the other hand, the scope of the skopobiota is to enhance the current microbiome and make it less susceptible to pathogen attacks, making it more resistant, rather than restoring the ‘healthy’ microbiome. It may operate as a buffer, making it less prone to changes.

The synergistic combined use of Blad/BCO application with inoculation with a selected consortium of endophytes to control/treat plant infections located in the stem/trunk and/or roots. This is a novel approach, meaning that it may be applied to all plant ailments (diseases, plagues and abiotic stressors).

The combined use of any of the above procedures with the X-ray tomographic methods described in the patents derived from the International Application No. PCT/EP2011/068320. This is also a novel approach, meaning that it may be applied to all plant ailments (diseases, plagues and abiotic stressors).

Knowing that Blad or the Blad-containing oligomer (BCO) is a contact (i.e. non-systemic) and non-translocable fungicide and taking into account the definitions provided above for biostimulants, it is clear that the effect of applying Blad or BCO at a site (e.g. leaves) which is far away from the site of infection (e.g. functional or non-functional xylem in trunk and roots, typically inaccessible even from a translocable and systemic pesticide point of view) results from a different type of disease control, in the present case involving modulation at the level of the microbiome. It can therefore be used in prevention as well as cure in several plant diseases. And furthermore, since the strengthening of the microbiome has been shown to induce resistance to abiotic stresses such as drought, Blad can promote resistance to both biotic and abiotic stresses.

Aspects Relating to Preferred Plant Types and Situations (for all Aspects Including Diagnostic and Treatment)

The invention provides non-destructive diagnostic methods and means to treat and/or control the plant diseases which affect both functional and non-functional xylem in both herbaceous and woody plants, as well as those affecting the roots. Preferred plants comprise imbalances in the composition of xylem/root microbiomes which in turn facilitate or promote xylem infection.

Preferred uses and methods which may be used alone or in combination, for example in synergistic combination, comprise a mode of action that is via a modification (change) of the resident microbiome:
the application of the non-translocable, contact fungicide Blad-containing oligomer (BCO) at a site (e.g. leaves) far away from the point of infection (e.g. trunk or root xylem tissues or roots, typically inaccessible from a pesticide point of view);
the application of the ‘skopobiota’, a neologism built from the Greek word skopos, which translates in ‘purpose’, representing a diverse array of selected microorganisms who may act synergistically in order to accomplish a predefined purpose in/on a specific environment, preferably in the sense that the multiple interactions among organisms will decrease the pathogen's likelihood or ability of successfully producing or continuing an infection in the plant. The organisms that constitute this skopobiota are typically endophytes which naturally occupy the xylem of that very same (healthy) plant species under analysis, to be used for biological control.

The plant may be part of an agricultural situation that involves the use of diverse formulations of compounds, substances and microorganisms which interfere positively with plants, not only promoting their growth but also inducing natural resistance to stresses. In this context, Blad and skopobiota are both adaptogens, with Blad acting indirectly on the plant microbiomes as a prebiotic-like agent, and the skopobiota acting directly as a probiotic agent. Not surprisingly, when applied to the same plant their activities are synergistic. Therefore, they are supposed to be responsible for the induction of the beneficial effects that result from fortified and strengthened microbiomes, positively favoring growth and protecting the plant from both biotic and abiotic stresses in a way which is (i) independent on the site and route of administration of the adaptogen, (ii) on the type of stressor and (iii) on the location of the stressor. Thus, for example, the adaptogen may be applied on the leaves with protection taking place in the out-of-reach inner woody tissues of roots, which cannot be accessed even with translocable, systemic compounds.

Situations in which the Invention May be Used

Since the discovery of antibiotics we seem to have reached, for the first time, a delicate balance, with humans and human-desired species on one side and human-unwanted organisms on the other. It is certain that we will never achieve full control of the situation. At best, we must continue our permanent struggle to maintain that equilibrium. The current and future emergence of apparently untreatable diseases (e.g. multidrug resistant, or MDR bacteria and the type of plant recalcitrant diseases addressed in the present application) only makes matters worse. Some of these issues raise serious concerns in our long-term struggle against unwanted organisms and on whether we will be (or not) successful.

The invention may be used to where the pathogens have resistance mechanisms against (phyto)pharmaceuticals by the target organisms in response to the (ab)use of pesticides/antibiotics.

The use of systemic pesticides in plants, albeit flowing from leaves to roots, does not reach all plant parts, most notably the inner areas of woody stems, trunk and roots (i.e. the xylem). Such tissues contain bioactive defensive compounds (e.g. stilbenoids in grapevine, isoprenoids in pine trees) but lack significant 02 levels and are essentially metabolically inert, meaning that they cannot trigger a coordinated biochemical response and are therefore basically defenseless with respect to any undesirable organism which manages to enter the woody tissues and which is unaffected by the bioactive compounds naturally present. Well known examples are disclosed below. These relate to preferred plants and/or preferred pathogens and/or preferred combinations of plants and pathogens for aspects of the invention.
The pine wood nematode Bursaphelenchus xylophilus, the causal agent of pine wilt disease on Pinus trees and its vectors, the insects Monochamus spp.;
The red palm weevil, Rhynchophorus ferrugineus, is a species of snout beetle. Its larvae can excavate holes in the trunks of palm trees up to 1 m long;
The fungal esca complex of diseases in grapevine (as well as several other grapevine wood diseases);
Advanced stages of infection of the oomycete Phytophthora cinnamomi in cork oak and in many thousands of other plant species, as well as other Phytophthora species;
The quick decline syndrome of olive trees and Pierce's disease in grapevine caused by the bacterium Xylella fastidiosa, which is known to affect over 300 host species, including fruit and forest trees, ornamental species and herbaceous crops.

The ability of these plant ailments to grow ‘silently’ (i.e. in the absence of visible symptoms during a considerable part of the infection/infestation) out of the reach of phytopharmaceuticals comprises the lead of the present application.

The asymptomatic nature of these plant diseases/plagues during the initial stages of infection/infestation and their ‘silent’ modes of dissemination apparently rule out the possibility of cure or eradication. Therefore, the proposed, strongly multidisciplinary strategies aim at using a combination of the same non-conventional approaches to drastically reduce and control the spread of all xylem-affecting ailments, which equally damage functional and non-functional xylem in both woody and herbaceous plants. This is made possible because regardless of the causal agent involved (bacteria, fungus, oomycete, insect, etc.), such ailments share several features in common. Three main pathosystems will be addressed in this application as examples of the xylem-affecting diseases. Many others exist, such as for example infection of kiwi (Actinidia deliciosa) by the fungus Phaeoacremonium minimum (Roberti et al, 2017), and cauliflower clubroot, the disease caused by the fungus-like microorganism Plasmodiophora brassicae on cauliflower, one of several vegetables in the species Brassica oleracea (Santos et al, 2017).

The esca complex of diseases of grapevine (Vitis vinifera), caused by a consortium of fungi (most often Phaeomoniella chlamydospora, Phaeoacremonium minimum and Fornitiporia mediterranea) which may act alone, in combination or in succession—Once an emerging risk, now a well-established problem in plant health in the EU, as well as in all viticultural regions of the world. For simplicity, in this proposal this complex of diseases will simply be referred to as esca. A proper nomenclature has been proposed by Surico (2009).

Cork oak (Quercus suber) die-back caused by the oomycota Phytophthora cinnamomi, which affects countless forest and agricultural species throughout the world—Once an emerging risk, now a well-established problem in plant health not only in the EU but worldwide. Over 5,000 plant hosts have been identified for P. cinnamomi, which allied to the oomycete capacity to survive adverse conditions make it an extremely serious plant pathogen (Erwin & Ribeiro, 1996; Hardham & Blackman, 2018). Cork oak is generally regarded as moderately susceptible to P. cinnamomi However, the pathogen seems to readily infect weakened plants (e.g. plants subjected to a severe drought), with flooding acting as a crucial step for spreading and disseminating P. cinnamomi spores underneath the ground. Currently, Quercus is a genus specially threatened by P. cinnamomi in the Mediterranean area and in California. The great importance of cork oak decline is highlighted in two letters of support (enclosed as separate pdf files) received by one of the applicant's collaborators (a cork producer) from two different European Commissionaires (Agriculture and Science).

Over 100 species have been described within the genus Phytophthora (Thines, 2013), the best known of which is P. infestans, the causal agent of mildew in potato and tomato plants. According to Erwin & Ribeiro (1996) and Agrios (2005), most survive in the soil for long periods in the absence of a host and affect the parts of the plant in contact with the soil, thus inducing the destruction of the root system. The impact of this organism is mirrored in its own name, as Phytophthora means, in Greek, plant destroyer. It is therefore a threat to many horticultural, fruit, forest and ornamental species (Erwin & Ribeiro, 1996; Agrios, 2005).

Phytophthora infestans infects many plants, among which are potato and tomato, the two most widely grown vegetable food crops in the world, where it causes the disease known as late blight or potato blight. P. infestans infects all aboveground parts of susceptible plants at any stage of plant development. Affected stems and petioles exhibit dark lesions and may collapse at the point of infection, leading to death of all distal parts of the plant (Nelson, 2008).

It is well known that Phytophthora species (e.g. P. cinnamomi) colonize the xylem and cause xylem discoloration and dysfunction. They are also known to attack the phloem and cambial tissues of trees, causing stem necrosis, and do so by spreading upwards from the roots or through aerial infections which penetrate wounds or the outer bark of the stem (Brown and Brasier 2007).

Olive quick decline syndrome (OQDS; in Italian: complesso del disseccamento rapido dell'olivo) in olive tree (Olea europaea), caused by the bacterium Xylella fastidiosa, which thrives in the plant xylem and is also responsible in the US for Pierce's disease (PD) in grapevine—A new risk in plant health not only in the EU but worldwide. X. fastidiosais a Gram—negative, slow growing and strictly aerobic bacterium in the family Xanthmonadaceae (Baldi and La Porta, 2017). Since the first report in grape in the US in 1973, X. fastidiosa has been identified in an increasingly large number of plant hosts, with or without symptoms, and recognized to be the causal agent of different diseases. X. fastidiosa can infect a great number of plant species. A partial list of the main hosts has been published in 2016 (EFSA, 2016). At present, according to the European Food Safety Authority (EFSA), the updated list of X. fastidiosa hosts consists of 359 plant species (including hybrids) from 75 different plant families (EFSA, 2016). Even if the infection process is always the same, the symptoms and the diseases caused by X. fastidiosa may vary among species. In many cases also wild plant species were found to carry this pathogen, but often in a latent stage only (Baldi and La Porta, 2017). EFSA reported recently that X. fastidiosa was detected in olive trees in Europe for the first time in Puglia, southern Italy (October 2013). Subsequently in 2015, it was detected in France and Corsica, then Germany and Spain and more recently Portugal. Besides grapevine, Quercus ilex (holm oak) and Citrus species are among other host plants for X. fastidiosa. Many sap-feeding insects can function as vectors for the transmission of X. fastidiosa to host plants.

The current rate of dissemination and spread of these diseases is extremely worrying, with a devastating potential impact ranging from agricultural production and forestry to human nutrition and trade (e.g. wine, cork and olive oil). Currently the situation in what concerns the three main pathosystems under study is far worse than the inexistence of effective treatment procedures. In the absence of appropriate diagnostic methods to screen the material leaving nurseries, infected plant material continues to leave nurseries for the establishment of plantations.

All the above pathogens, plants, types of situation, types of plant damage are included in different aspects of the invention, including diseases caused by fungi, oomycota or by a bacterium.

The invention includes treating xylem-affecting ailments, regardless of the nature of the causal agent. The invention includes the following situations:
ailments (diseases) that affect the host xylem.
pathogens that naturally protected from the application of commercially available pesticides, for example they infect the woody tissues, with optionally Phytophthora also destroying the fine roots.
X. fastidiosa grapevine and olive tree infections,
situations cause by esca or Phytophthora. These were recognized many years ago, but it was not until the 80s that the spread and dissemination of all of them ‘exploded’. The reasons behind this are not clearly understood but are believed to result from a combination of factors which affect negatively the host plants. A number of them have been proposed, such as climate change, intensive application of pesticides, systematic grafting, wounding induced by pruning or cork removal, reduced precipitation, poor soils, thin soils, lack or excess of organic matter, overgrazing, other changes in agricultural and forest management practices, etc. The role of the wood (and soil) microbiome cannot be ignored.

The invention relates to plants which have an absence of external symptoms during the early stages of the infection. Nurseries are a prime port for xylem-related disease dissemination since the infection processes evolve through a more or less lengthy phase in the absence of externally visible symptoms. As a result, because infected plants appear externally healthy, these diseases have been considered as silent threats, allowing not only infected plants to leave nurseries, but also their man-driven subsequent dissemination. The pathogens exhibit typically a reduced growth rate and the external symptoms manifest themselves late, preventing any measure of control. As a result, many plant hosts may be infected and asymptomatic, suffering a disguised but very deadly evolution. In the case of esca, for example, when foliar symptoms become visible it is usually rather late in the stage of disease progression. Nevertheless, symptoms in one plant may appear in one season and disappear in the next while the plant continues critically ill. On the other hand, only a minority of the infected plants will often exhibit externally visible (e.g. foliar) symptoms, meaning that the presence of these is by no means a good indicator of the sanitary state of the vineyard. A study was conducted in a vineyard in the river Tejo valley, Portugal, with virtually no foliar symptoms at all. Several hundred wood core rods were removed with a gimlet from various positions along the trunk and arms of the vines for preliminary metabarcoding analyses. All wooden samples exhibited internal symptoms of esca disease infection.

Preferred plants are in nurseries, which are a prime target to control at least in part disease dissemination and spread. Three main routes are used in the spread and dissemination of the xylem affecting ailments:

1—Via nurseries. Farmers are currently establishing vineyards, cork oak forests and olive tree orchards using infected material. A study conducted recently showed that nurseries in Europe are selling vine grafts and cuttings with a proportion of material infected with esca-related fungi ranging from 10 to 70%. Several European (including Portuguese) farmers have won cases taken to courts against nurseries for selling them infected material. Unfortunately, all current commercially available screening methods for infected vine grafts and cuttings are destructive.

2—Via open wounds. Of major concern here are the pruning wounds in grapevines and olive trees, as well as cork removal and ploughing the soil of cork oak forests, thus damaging/wounding the superficial part of the root system—Something which may become serious in thin soils with hard rock near the surface. Such practices not only constitute a port for the entry of pathogens, but also weaken the plants in their chemical struggle against pathogens, especially if performed at a time the plants are not metabolically active. Developing suitable pruning wound protection strategies will most probably arise in the near future. Preferred plants have any of these types of wounds or damage.

3—As a result of both biotic and abiotic stresses. Wind, rain and insects, for example, have been implicated in esca fungi diffusion, flooding in the spread of Phytophthora underground, and insects (e.g. Philaenus spumarius) in the dissemination of Xylella. Preferred plants have been impacted by any of these agents.

Therefore, for grapevine esca, cork oak decline and olive quick decline syndrome, it would be of utmost importance the early detection of the externally asymptomatic presence of the pathogens, thus allowing the implementation of a timely and effective eradication plan. This should be done under a two-stage control/screening program: the first one, at the level of nurseries; subsequently, at a later stage, attempted extension to field conditions, i.e. vineyards, cork oak forests and olive tree orchards.

There is currently a feeling that although the pathogenic agents have been apparently well identified, other factors, or a combination of them, other than the pathogens seem to trigger the infection process. It is as though the combination of a number of factors would pre-dispose the hosts for infection, facilitating the conditions for disease establishment.

In the absence of proper diagnostic methods capable of detecting the early stages of these diseases (i.e. grapevine esca, cork oak dieback and olive quick decline syndrome), infected material will continue to leave nurseries, greatly contributing to their dissemination. This situation evolves negatively later on since there are no effective measures for treatment. Overall and under field conditions, the pathogens involved will spread ceaselessly. These species of plants and these conditions are preferred.

Therefore, the ultimate objective is not to try the out-of-reach eradication of these diseases, but rather to improve methods and strategies for their prevention and containment, as well as to enlarge the range of tools for integrated and sustainable disease management. Such procedure(s) may be subsequently extended/adapted to agricultural and forest conditions.

In the present application, novel approaches are presented, which optionally together with the X-ray based technique proposed in PCT International Patent Application No. PCT/EP2011/068320 and a number of cautious practices which are known to influence negatively the plant or wood microbiome (e.g. spraying open wounds with copper-based fungicides) may be combined to detect, prevent and contain the spread of the xylem-affecting ailments, first at the nursery level, then at the agricultural/forestry level. Interestingly, all these approaches, which have all produced excellent preliminary results on esca, do not target the pathogen itself but rather the host or, more accurately, the pathosystem. Such procedures are inevitably strongly multidisciplinary, involving phytopathology, plant physiology, biochemistry, proteomics, metagenomics and metabarcoding, microbiology and X-ray tomography. In this way, a group of common strategies are proposed to target different types of pathogens (i.e. all xylem affecting agents) that are responsible for emergent recalcitrant diseases in plants and which are assuming great economic importance not only in Europe but also worldwide, as there are neither known treatments available, nor prospects for developing them.

The invention relates to diseases and plagues which affect both the plant non-functional and functional xylem. Such diseases are disseminating ‘silently’, apparently unrestrictedly, throughout the world at an unprecedented rate. They are out of the reach of the human's most efficient phytopharmaceuticals, and no prospects exist for the development of a cure or effective treatment and control capable of reversing their apparently endless progression. They have already reached (but continue to increment) the status of huge economic importance. There is no immediate hope to develop a cure that will heal the infected plants. One objective of the present invention is therefore to use the same group of non-conventional approaches in an attempt to develop an efficient strategy to drastically reduce their apparently unstoppable rate of dissemination, as well as controlling the diseases/plagues in already infected/infested plants.

The specific diseases addressed as examples in the present invention constitute an extremely serious situation on their own. Esca has been considered as the ‘Phylloxera of the 21st century’, but its consequences are likely to be far worse, since it is a worldwide problem and no Vitis species are known to exhibit resistance to it, meaning that grafting is not an option. The prospects of viticulture are therefore rather dark.

A preferred aspect of the invention relates to Phytophthora which is killing cork oaks and chestnut trees in Portugal as well as in Southern Europe in general, and there's nothing we can do about it. But Phytophthora-causing problems do not end here. In fact, Phytophthora cinnamomi is one of the most devastating plant pathogens in the world. It infects close to 5,000 species of plants, including many of importance to agriculture, forestry and horticulture. The inadvertent introduction of P. cinnamomi into natural ecosystems, including several recognized Global Biodiversity Hotspots, had disastrous consequences for the environment and the biodiversity of flora and fauna (Hardham & Blackman, 2018). In addition, the genus Phytophthora includes over 100 species, which infect a very large number of plants.

The invention relates to Phytophthora, for example in the cork oak ‘montado’. The invention also relates to Xylella fastidiosa infection of olive trees that may derive from infected nursery material or arise under orchard conditions due to bacterial dissemination by the insect vectors. In either case, we presently have neither non-destructive diagnostic methods nor ways of efficiently reducing disease spread or of treating infected plants. Under the present conditions, it seems likely that X. fastidiosa infection will disseminate in a never-ending mode and that it will eventually propagate to infect some or many other species.

In all these cases, the problem starts with already infected new plantations, since in the absence of appropriate diagnostic methods, unscreened infected plant material is permanently leaving nurseries—Once again, there is nothing we can do about it at the moment, since there are neither proper non-destructive control and/or screening methods nor compulsory legislation to do so.

Current Situation

Many of the xylem-affecting diseases have been known for many years but it was not until the end of the 20^{th} century that they assumed a considerable economic importance. Esca of grapevine and Phytophthora in cork oak, for example, ‘exploded’ from the eighties to the present day and all available evidence points that they will continue to do so.

There is neither a way to treat them nor prospects of developing efficient methods to control them. As they typically do not show externally visible symptoms until the infection has progressed to an advanced stage, disease dissemination takes place freely, not only naturally, but is actually accelerating due to man-derived activities. A considerable proportion of the plants which leave nurseries to establish new vineyards and cork oak forests are already infected. Under field conditions, a few vectors are involved in ‘esca spores’ dissemination including wind, rain, insects and viticultural practices, whereas those of Phytophthora spread by underground water. These specific situations and species are included in the present invention.

It is not known why many plant diseases affecting the xylem are disseminating, spreading and developing at such alarming rates. The combination of a number of factors has been suggested, such as climate change, intensive application of pesticides, systematic grafting, wounding induced by pruning and cork removal, reduced precipitation, poor soils, thin soils, lack or excess of organic matter, overgrazing, other changes in agricultural and forest management practices, etc. However, no direct evidence has been presented to support such claims. Preferred aspects of the invention relate to plants in such situations.

Recent evidence obtained by the applicant's research group (Plants for Health & Nutrition, LEAF, Instituto Superior de Agronomia, University of Lisbon, Lisbon, Portugal) points to imbalances in the natural wood microbiome as the immediate cause for plant xylem sensitivity to disease. These imbalances may, in turn, result from a combination of several factors, such as some of those referred above, which may vary from pathosystem to pathosystem. In the case of grapevine trunk diseases (GTDs), for example, a gradual change in the wood microbiome caused by successive and intensive fungicide applications may lie behind esca and other diseases development, eventually converting some of the most resistant microorganisms from endophytes to pathogens and/or facilitating the de novo entry of pathogens. A number of indirect observations support the applicant's hypothesis: in many cases wild plant species were found to carry X. fastidiosa, but often in a latent stage only (Baldi and La Porta, 2017); in contrast to what would be expected, preliminary experiments performed in the applicant's lab showed that grapevine pruning wounds treated with copper-based fungicides and subsequently inoculated with Phaeomoniella chlamydospora led to a more intense wood symptomatology than the control, an observation that was attributed to differential sensitivity of the wood endophytes to the fungicide, thus imposing an imbalanced microbiome—Much in the same way to what happens to our gut microbiome when we ingest a broad spectrum antibiotic such as, for example, penicillin.

Therefore, the inventors see that the plant xylem microbiome plays an essential role in the homeostasis of a healthy plant and that factors that cause an imbalance are often involved in promoting the development of many plant diseases which currently rage out of control.

The Diagnostic and Treatment Aspects of the Invention

This invention provides diagnostic and disease control procedures (e.g. inoculation with a skopobiota, a suitable combination of plant xylem endophytes, and/or the application of Blad-containing oligomer, a non-translocable, contact fungicide and plant biostimulant at a site, e.g. leaves, far away from the point of infection, e.g. trunk or root xylem tissues, typically inaccessible from a pesticide point of view), while preferably avoiding the use of practices which imbalance both the functional and non-functional xylem microbiomes in an attempt to non-destructively diagnose the initial stages of infection of xylem-related plant ailments and to maintain or contribute to a healthy, homeostatic xylem population of endophytes. Such procedures may reduce drastically the percentage of infected material leaving nurseries, thus allowing the introduction of a seal of quality capable of ensuring that no more than x % plants are infected. Therefore, the ultimate objective is not to try the out-of-reach eradication of these xylem-related diseases, but rather to improve methods and strategies for their prevention and containment, as well as to enlarge the range of tools for integrated and sustainable disease management. Such procedure(s) may be subsequently extended/adapted to agricultural and forest conditions.

The Typing and Manipulation of the Microbiome

From a scientific point of view, ‘microbiota’ has been defined as the total set of microbes found within a specific environment, whereas the term ‘microbiome’ means the total collection of microbial genomes encountered in a particular environment, with ‘microbe’ defined as any multi or unicellular organism which is microscopic. However, over the years researchers have started to use the words ‘microbiota’ and ‘microbiome’ interchangeably. The present invention applies to any such population of microorganisms colonizing the plant, for example which are present in or on the plant. The microorganism population may be present in any tissue, for example any specific tissue disclosed herein.

Unlike animals, where the bacterial pathogens and bacteria-derived diseases outnumber those caused by fungi, most plant diseases are caused by pathogenic fungi. Thus, 120 genera of fungi, 30 types of viruses and eight genera of bacteria are responsible for the ca. 11,000 diseases that have been described in plants (Ferreira et al, 2006). In this context, it is convenient to define ‘mycobiome’ as the total set of fungal species found within a specific environment.

The skopobiota may comprise a diverse array of selected microorganisms who may act synergistically in order to accomplish a predefined purpose in/on a specific environment.

The invention includes use of skopobiotas to restore diversity in a system (e.g. depleted soils, reclaimed environments from pollution) or to be exploited in the control of plant pathogens, as this application indicates. In one aspect the invention increases the number of different microorganism species colonizing the plant, for example by at least 10%, 20%, 50%, 80%. The number of species colonizing the plant may be increased by at least 5, 10, 20, 30 or 50.

It should be born in mind that the vast majority of microbiome studies performed to date involve bacteria. Comparatively, very few studies have been performed on fungal microbiomes (or mycobiomes) and, among these, almost none when the functional or non-functional plant xylem is concerned.

The microbes that colonize the different plant organs may be considered as the plant's second genome. Due to the importance of the soil habitat of plants, the majority of research focuses on the rhizosphere, even though microorganisms are also able to colonize most plant tissues (Berg et al, 2014). The present invention relates to the rhizosphere or to non-rhizosphere populations of microorganism colonizing plants.

It is believed that plant microbiomes, characterized by unique assemblages of microorganisms, contribute to nutrition, development, health and overall fitness of their plant hosts (Friesen et al, 2011). In other words, if we take competition and pathogenicity aside, interactions between plants and microbes may benefit plants by increasing the acquisition of nutrients, producing growth hormones, and defending against enemies.

Most studies on plant microbiomes have focused on the rhizosphere (microbes surrounding roots) and the phyllosphere (microbes colonizing the surfaces of the aerial habitats of plants)—Microbes termed epiphytes. Few studies have considered the endosphere (microbes within roots) (Bulgarelli et al, 2012; Lundberg et al, 2012) and the wood microbiomes—Microbes termed endophytes. The microbial communities transition from the outside to the inside of the root forms a soil-root continuum, comprising the bulk soil not affected by root activity, rhizosphere (the soil microenvironment immediately surrounding the root), rhizoplane (the root surface), and endosphere (the root interior) (Van Der Heijden & Schlaeppi, 2015). The specific composition of these compartments depends on the specific soil-associated microorganisms and the host, with particular reference to the plant genotype and its physiological status. Thus, for example, rhizosphere microorganisms and endophytes can help host plants to adapt to various adverse environments (Nogales et al, 2016), and Fitzpatrick et al (2018) provided evidence suggesting that the root microbiome influence plant performance in response to variation in biotic and abiotic components of the environment. Drought shifts the composition of root microbial communities in numerous grass species. However, whether variation in the diversity or composition of host plant root microbiota contributes to plant drought tolerance is unknown (Santos-Medellin et al, 2017). Also, plants can indirectly compete with one another through recruitment of soil microbes (Bever, 1994).

Using isogenic Arabidopsis thaliana mutants with altered immune systems in a wild soil and recolonization experiments with synthetic bacterial communities Lebeis et al (2015) observed that biosynthesis of the foliar defense phytohormone salicylic acid is required to assemble a normal root microbiome, i.e. salicylic acid modulates colonization of the root by specific bacterial families.

The Wood Microbiome

Rather than being sterile, the wood of healthy plants is home to a diverse microbiome. There is increasing evidence that microorganisms inhabiting the heartwood tissues (essentially composed of dead xylem cells) of woody plants may have great importance but to date such studies have been unfairly neglected. Most experiments have been centered on individual bacterial or fungal isolates and only a few studies have been extended to microbial consortia. Preferred aspects of the invention relate to the wood microbiome.

So far, wood microbiomes have been generally considered as composed of simple communities within woody tissues. Additionally, many of the fungal genera identified seem to overlap with those commonly found within leaf and root endophyte habitats. They have been considered as essentially dependent on the plant genotype, but conditions that are unusual in what the rhizosphere and the phyllosphere are concerned may evolve frequently, such as lack of oxygen, with the anaerobic conditions favoring fermentation or even methanogenesis, as well as the potential to fix nitrogen, as evidenced by acetylene reduction assays (Hacquard & Schadt, 2014).

The applicants have also found evidence suggesting that wood microbiomes of different plants, either from the same species or not, may ‘exchange’ microbial species among them. A prerequisite may be required, which is the presence of open wounds in the woody tissues, a condition facilitated in vineyards by the wounds inflicted every year by pruning. The applicant's evidence was based on the observation that the wood mycobiome of a specific vineyard contained unusual fungi in what grapevine is concerned, but whose normal hosts could be found in its immediate surroundings (characterized by the presence of diverse vegetation), within a 25 m radius from the perimeter. Most of those species are listed in Table 1. Table 1 discloses preferred phyla, classes, orders, families, genera and species of plants which may exchange wood microorganisms for the invention.

Table 1. Classification of the woody plants encountered in the proximity of the vineyard used in the present study (Almotivo vineyard, Instituto Superior de Agronomia, University of Lisbon, Lisbon, Portugal).

| Phylum | Class | Order | Family | Genus | Species |
|---|---|---|---|---|---|
| Magno- | Liliop- | Liliales | Agavaceae | Agave | A. americana |
| liophyta | sida | | | | |
| — | — | — | Aspho- | Aloe | A. |
| | | | delaceae | | arborescens |
| — | Magno- | Apiales | Pitto- | Pitto- | P. undulatum |
| | liopsida | | sporaceae | sporum | |
| — | — | Caryo- | Cactaceae | Opuntia | O. ficus- |
| | | phyllales | | | indica * |
| — | — | — | — | — | O. stricta |
| — | — | Fabales | Fabaceae | Ceratonia | C. siliqua |
| — | — | — | — | Cercis | C. |
| | | | | | siliquastrum |
| — | — | Fagales | Fagaceae | Quercus | Q. |
| | | | | | rotundifolia |
| — | — | Lamiales | Oleaceae | Fraxinus | F. |
| | | | | | angustifolia |
| — | — | — | — | — | F. australis * |
| — | — | — | — | Olea | O. silvestrys * |
| — | — | — | — | Phillyrea | P. latifolia * |
| — | — | — | — | — | P. media * |
| — | — | Myrtales | Myrtaceae | Eucalyptus | E. camal- |
| | | | | | dulensis |
| — | — | — | — | — | E. globulus |
| — | — | Proteales | Platanaceae | Platanus | P. |
| | | | | | occidentalis |
| — | — | Rhamnales | Rhamnaceae | Rhamnus | R. alaternus |
| — | — | Rosales | Rosaceae | Eriobotrya | E. japonica |
| — | — | — | — | Prunus | P. dulcis |
| — | — | — | — | — | P. persica |
| — | — | — | Moraceae | Maclura | M. pomifera |
| — | — | Urticales | Ulmaceae | Celtis | C. australis * |
| Pino- | Pinop- | Pinales | Cupres- | Cupressus | C. |
| phyta | sida | | saceae | | sempervirens |
| — | — | — | Pinaceae | Pinus | P. pinea |

(*) Denotes the most abundant species in the proximity of the Almotivo vineyard.

The identification of sequences in the applicant's dataset revealed an unprecedented diversity for the grapevine wood mycobiome, as assessed by metabarcoding using Illumina® next-generation sequencing (NGS) techniques. Taxa that were assigned to genus or species level are 289, 50 of them are found in relative abundance (RA) greater than 0.1%, while the remaining 239 are considered rare taxa (RA<0.1%). Within these 239 taxa, 146 are found in a RA included between 0.1 and 0.01%, and 93 have a RA lower than 0.01%. The full list of taxa is available in Table 2, and these are preferred organisms for typing in the invention or including in a skopobiota.

The qualitative overview of the wood mycobiome will focus mainly on the 30 most abundant taxa in perennial wood (PW) and the 12 most abundant in annual wood (AW), which account for 79.1 and 80.8% of the total RAs respectively (Table 3), while the remaining percentages represent unidentified taxa or fungi found in lower abundances. Within this group of taxa, five genera and nine species of fungi are described for the first time in association with the grapevine wood mycobiome, while the remaining 18 taxa have already been reported (Table 3).

The community encountered in perennial wood is characterized by the presence of both ascomycetes and basidiomycetes (66.7% and 27.7% RA), with high abundances of tracheomycotic pathogen Phaeomoniella chlamydospora (25.8%) and white rot agent Fornitiporia sp. (14.6%), two organisms directly associated with esca proper and other esca-related syndromes. Among all sampled wood cores (n=80) P. chlamydospora was present in 68 of them (85%; RA>0.1%), while Fornitiporia sp. in 58 (64%; RA>0.1%) or 14 (17.5%; RA>35%). Other GTD pathogens among the 30 most abundant taxa are Eutypa lata (0.7%) and E. leptoplaca (0.9%), within the Diatrypaceae. More members of this family are Anthostoma gastrinum (0.9%), a potential wood pathogen, as well as E. flavovirens, Eutypella citricola and Cryptovalsa ampelina, identified as rare taxa. Members of the Botryosphaeriaceae (e.g. Diplodia pseudoseriata, Neofusicoccum parvum, N . australe), Ilyonectria sp. and Neonectria sp. are also found, although represented only as rare taxa (Table 2). Decay agents, such as Fornitiporia sp., Fornitiporia mediterranea (0.2%) and Inonotus hispidus (0.3%), were also identified in this study, along with several others represented in minor abundances (e.g. Fornitiporella sp.). Among the endophytes or saprophytes, Alternaria sp. (3.2%), Cladosporium sp. (1.9%), Aureobasidium pullulans (0.4%) and Psathyrella sp. (0.5%) are the most abundant. Several other genera or species, identified for the first time is association with grapevine wood, amount to 14 taxa out of the 33 most abundant in PW or AW (Table 3). Table 3 discloses preferred organisms for any aspect of the invention.

Annual wood is also colonized by both ascomycetes (76.3%) and basidiomycetes (18.8%). The most abundant taxa are endophytic and saprophytic fungi, with Alternaria sp. (14.6%), Ramularia sp. (9.4%) and Cladosporium sp. (8.2%) being among most abundant, as well as other species reported for the first time (e.g. Debaryomyces prosopdis; Table 31. Only two wood pathogens are encountered in annual wood, namely P. chlamydospora (3.9%) and Diaporthe sp. (0.8%), while other pathogenic agents are found in minor abundances (RA<0.2%; e.g. Neofusicoccum australe).

The core mycobiome, namely the taxa shared between PW and AW, is constituted by 44 taxa. Only 10 taxa are unique to AW and the remaining 235 are unique to PW. All the 10 unique taxa found in AW are considered rare taxa, as their RAs are lower than 0.1% of the total, while among the many taxa unique to PW we find organisms belonging to the Hymenochaetaceae, Lophiostomataceae, Pleomassariaceae, Xylariaceae and several others (Table 2).

Table 2. List of taxa identified by metabarcoding using using Illumina® next-generation sequencing (NGS) techniques to genus or species level present in the dataset including all sampling points for all tissue types—objective (1)—. Three groups are created to separate taxa present in a relative abundance (RA) greater than 0.1%, included between 0.1 and 0.01%, lower than 0.01%. The taxa with a RA<0.1% are considered rare taxa. Taxa followed by (*) are part of the core mycobiome—shared by permanent wood and annual wood—, taxa followed by (†) are unique to annual wood, taxa not followed by any symbol are unique to permanent wood. The taxa and species shown in Table 2 are preferred organisms for the skopobiota of the invention.

| | | |
|---|---|---|
| RA > 0.1% (n = 50) | Cyphellophora sp. | Xanthoria sp. |
| Acremonium sp. | Cystobasidium | Xylodon sambuci |
| Acremonium | pinicola | Zymoseptoria sp. |
| alternatum* | Cystobasidium sp. | RA < 0.01% (n = 93) |
| Alternaria sp.* | Cystofilobasidium | Acremonium fusidioides |
| Angustimassarina | capitatum | Agaricus blazei |
| acerina | Cystofilobasidium | Alternaria brassicae |
| Anthostoma | macerans | Alternaria eureka |
| gastrinum | Devriesia | Alternaria |
| Aureobasidium | pseudoamericana* | metachromatica |
| pullulans* | Devriesia sp. | Apiotrichum domesticum |
| Biatriospora | Dioszegia | Arthrinium sp. |
| mackinnonii | hungarica | Arthrobotrys superba |
| Candida friedrichii* | Erythrobasidium | Ascochyta |
| Capronia coronata | hasegawianum | medicaginicola |
| Cladosporium sp.* | Eucasphaeria | Aspergillus amstelodami |
| Clonostachys rosea | capensis* | Aspergillus ochraceus |
| Colletotrichum sp.* | Eutypa flavovirens | Beauveria bassiana |
| Cryptococcus sp.* | Eutypella citricola | Bjerkandera adusta |
| Cryptococcus | Exidia japonica | Candida etchellsii |
| heimaeyensis | Exobasidium sp. † | Candida mycetangii |
| Cryptococcus | Exophiala | Candida zeylanoides |
| victoriae* | oligosperma | Capronia pulcherrima |
| Debaryomyces sp.* | Fellomyces sp. | Catenulostroma |
| Debaryomyces | Fomitiporella sp. | hermanusense |
| prosopidis* | Funneliformis | Cladosporium salinae |
| Diaporthe sp.* | geosporum | Colacogloea sp. |
| Eutypa lata | Fusarium poae | Cryptococcus aureus |
| Eutypa leptoplaca | Fusarium solani | Cryptosphaeria |
| Exophiala sp. | Ganoderma | subcutanea |
| Exophiala | australe | Cryptovalsa ampelina |
| xenobiotica | Ganoderma | Curvularia sp. |
| Filobasidium | lucidum † | Curvularia tsudae |
| globisporum | Ganoderma | Cyphellophora europaea |
| Filobasidium | resinaceum | Cyphellophora reptans |
| magnum* | Gibellulopsis | Cystofilobasidium |
| Fomitiporia sp. | chrysanthemi | infirmominiatum |
| Fomitiporia | Holtermanniella | Cytospora sp. |
| mediterranea | takashimae | Debaryomyces |
| Fusarium sp.* | Hyphodontia | mycophilus |
| Glomerella | alutaria | Dioszegia zsoltii var. |
| acutata* | Hyphodontia | yunnanensis |
| Guehomyces | radula* | Diplodia pseudoseriata |
| pullulans | Knufia epidermidis | Engyodontium album |
| Inonotus hispidus | Knufia perforans | Epicoccum pimprinum † |
| Lopadostoma | Laetiporus | Erysiphe necator |
| meridionale | sulphureus | Erythrobasidium |
| Lopadostoma | Leprocaulon sp. † | elongatum |
| quercicola | Meira nashicola* | Erythrobasidium sp. |
| Lophiostoma | Microdiplodia sp. | Exophiala bergeri |
| cynaroidis | Minimedusa | Fellomyces penicillatus |
| Lophiostoma sp. | polysp.ora | Fellomyces polyborus |
| Lophiotrema rubi | Mortierella sp. | Filobasidium wieringae |
| Malassezia | Mortierella | Fuscoporia ferruginosa |
| globosa* | minutissima | Gymnopus barbipes |
| Malassezia | Mrakia sp. | Hannaella sp. |
| restricta* | Mucor sp. | Hanseniaspora sp. † |
| Massarina sp. | Mycena metata | Heterobasidion |
| Meyerozyma | Naganishia | irregulare † |
| guilliermondii* | albidosimilis | Hyphoderma |
| Mycosphaerella | Neodevriesia | nudicephalum |
| tassiana* | capensis | Ilyonectria Iiriodendri |
| Penicillium sp.* | Neoerysiphe | Itersonilia pannonica † |
| Peniophora sp.* | galeopsidis | Knufia tsunedae |
| Phaeomoniella | Neofusicoccum | Kondoa aeria |
| chlamydospora* | sp.* | Kurtzmanomyces sp. |
| Psathyrella sp. | Neofusicoccum | Lachancea |
| Ramularia sp.* | parvum | thermotolerans |
| Rhinocladiella sp. | Neofusicoccum | Lycoperdon ericaeum |
| Rhodotorula | australe* | Magnaporthe grisea |
| mucilaginosa* | Neonectria sp. | Malassezia sp.* |
| Sporidiobolus sp. | Occultifur sp. | Malassezia sympodialis |
| Trematosphaeria | Orbilia sp. | Mariannaea |
| pertusa | Papiliotrema | superimposita |
| Vishniacozyma | flavescens | Mollisia cinerea |
| carnescens | Paraconiothyrium | Monographella |
| RA < 0.1% | sp.* | cucumerina |
| (n = 146) | Paraphaeosphaeria | Monographella |
| Absidia sp. | parmeliae | nivalis |
| Acremonium | Penicillium | Mortierella |
| brunnescens | citreonigrum | alpina |
| Annulohypoxylon sp. | Peniophorella | Mucor |
| Apiotrichum sp. | pubera* | hiemalis |
| Articulospora sp. | Petriella sp. | Mucor |
| Ascobolus sp. | Phacidiella | saturninus |
| Aspergillus sp. | eucalypti | Naganishia |
| Aspergillus conicus | Phaeomoniella sp. | albida |
| Aspergillus | Phallus impudicus | Naganishia |
| penicillioides* | Phanerochaete sp. | randhawae |
| Aspergillus | Phialemoniopsis | Neodevriesia simplex |
| proliferans | ocularis | Occultifur externus |
| Bensingtonia sp. | Phialophora | Papiliotrema |
| Bipolaris sp. | cyclaminis | pseudoalba |
| Blumeria graminis | Phialophora | Paraphoma fimeti |
| Boeremia exigua | verrucosa | Parasola |
| Buckleyzyma sp. | Physcia sp. | conopilus |
| Caloplaca | Pleospora fallens | Periconia |
| obscurella | Podospora sp. | pseudobyssoides |
| Candida sp. | Pyrenochaeta sp. | Periconia sp. |
| Candida | Pyrenochaeta | Phialemoniopsis |
| palmioleophila | keratinophila | curvata |
| Candida | Pyrenochaeta | Phlebia |
| parapsilosis | unguis-hominis | acerina |
| Candida sake | Pyrenophora | Phlebiopsis |
| Candida tropicalis* | tritici-repentis | gigantea † |
| Candida | Ramicandelaber | Pseudocercospora |
| orthopsilosis | sp. | sp. |
| Capnodium sp. | Ramichloridium | Ramularia |
| Capronia sp. | cucurbitae | stellenboschensis † |
| Cenococcum sp. | Rhizomucor | Rhodotorula |
| Ceratobasidium | pusillus | ingeniosa |
| cornigerum | Rhizopus | Rhodotorula |
| Ceratobasidium sp. | microsp.orus | terpenoidalis |
| Circinotrichum | Rhodotorula sp. | Rhodotorula |
| maculiforme* | Rhodotorula | toruloides |
| Citeromyces | graminis | Sclerostagonospora |
| matritensis | Rhodotorula | cycadis |
| Cladophialophora | nothofagi | Scopuloides rimosa |
| chaetospira | Rhodotoruia | Setophaeosphaeria |
| Cladophialophora | diobovata | badalingensis |
| sp. | Saccharomyces | Sistotremastrum |
| Cladosporium | cerevisiae | guttuliferum |
| delicatulum* | Sakaguchia | Sterigmatomyces |
| Cladosporium | dacryoidea | halophilus |
| fusiforme | Sarcoporia sp. | Taphrina |
| Cladosporium | Sarocladium | deformans |
| sphaerospermum* | subulatum* | Trametes |
| Clathrus ruber | Scheffersomyces | versicolor |
| Clitopilus sp. | spartinae | Trichosporon |
| Colletotrichum | Schizophyllum | asahii |
| acerbum* | commune | Uncispora |
| Colletotrichum | Sclerostagono- | sinensis |
| gloeosporioides* | spora sp. | Valsaria |
| Coprinellus | Scytalidium sp. | insitiva |
| micaceus | Sistotremastrum | Verruco- |
| Cryptococcus aerius | sp.* | cladosporium |
| Cryptococcus frias | Solicoccozyma | dirinae |
| Cryptococcus | terrea | Yamadazyma |
| uniguttulatus | Sporobolomyces | triangularis |
| Cutaneo- | oryzicola | |
| trichosporon sp. | Stemphylium sp. | |
| Cutaneotricho- | Tetracladium sp. | |
| sporon | Torulaspora | |
| cyanovorans | delbrueckii | |
| Cyberlindnera | Trametes hirsuta | |
| jadinii | Trichaptum | |
| | abietinum | |
| | Trichoderma sp.* | |
| | Trichoderma | |
| | harzianum † | |
| | Veronaea | |
| | compacta | |
| | Verticillium sp.* | |
| | Wallemia sp. | |
| | Wallemia muriae* | |

Table 3. List of most abundant taxa, identified by metabarcoding using using Illumina® next-generation sequencing (NGS) techniques to genus or species level, found in the grapevine wood of Almotivo vineyard. The list includes the 30 most abundant taxa found in permanent wood and the 12 most abundant in annual wood, for a total of 32 taxa. The numbers between brackets represent the relative abundance of that Phylum or Family in the permanent wood or annual wood (PW %-AW %) based on the table created to address objective (1). The ecology of the identified taxa in wood of grapevines or of other plants is shown based on available literature (E=endophyte, 5=saprophyte, P=pathogen, na=unknown ecology). The presence of taxa in different tissue types is based on the table created to address objective (2), (+) indicates presence (RA≥0.1%), (−) indicates absence or presence in RA<0.1%. The phyla, families and species of the organisms of Table 3 are preferred organisms for the skopobiota of the invention.

| | | | Relative abundance | | Ecology in | Presence in different tissue type | | | |
|---|---|---|---|---|---|---|---|---|---|
| Phylum | Family | Species | PW | AW | wood ¹ | GU/T/UT | A1/A2 | S1/S2 | C |
| Ascomycetes | Biatriosporaceae (0.6-0) | Biatriospora mackitmonii‡ | 0.6 | — | E ^{a} | −/−/− | −/− | −/− | − |
| (66.7-76.3) | Bionectriaceae (0.4-0) | Clonastachys rosea | 0.4 | — | E, S, P ^{b} | −/−/− | −/− | −/+ | − |
| | Davidiellaceae (2 0-8.2) | Cladosporium sp. | 1.9 | 8.2 | E, S ^{b} | +/+/+ | +/+ | +/− | + |
| | Diaporthaceae (<0.1-0.8) | Diaporthe sp. | <0.1 | 0.8 | E, S, P ^{b} | −/−/− | −/− | −/− | + |
| | Diatrypaceae (2.6-0) | Anthostoma gastrinum | 0.9 | — | S, P ^{c, d} | +/+/+ | +/+ | +/− | − |
| | | Eutypa lata | 0.7 | — | P ^{b} | −/−/− | −/− | −/+ | − |
| | | Eutypa leptoplaca | 0.9 | — | P ^{b} | −/−/+ | −/− | +/+ | − |
| | Dothioraceae (0 4-4.0) | Aureobasidium pullulans | 0.4 | 4.0 | E, S ^{b} | −/+/+ | +/+ | −/− | + |
| | Glomerellaceae (<0.1-0.7) | Colletatrichum sp. | <0.1 | 0.4 | P ^{b} | −/−/− | −/− | −/− | − |
| | Herpotrichiellaceae (27.1-3.9) | Exophiala xenobiotica† | 0.5 | — | na | −/−/− | −/+ | +/+ | − |
| | | Phaeomoniella | 25.8 | 3.9 | P ^{d} | +/+/+ | +/+ | +/+ | + |
| | | chlamydospora | | | | | | | |
| | Hypocreales (0.3-0.2) | Acremonium sp. | 0.2 | — | E ^{e} | −/−/− | −/− | −/+ | − |
| | Lophiostomataceae (3.8-0) | Angustimassarina acerina† | 0.5 | — | S ^{f} | −/−/+ | +/+ | +/+ | − |
| | | Lophiostoma sp. | 2.7 | — | E, S ^{b} | +/+/− | +/− | +/− | − |
| | | Lophiostoma cynaroidis† | 0.3 | — | E ^{g} | −/+/− | −/− | −/− | − |
| | Massarinaceae (0.5-0) | Massarina sp. | 0.5 | — | E ^{h} | +/+/+ | −/− | +/− | − |
| | Mycosphaerellaceae (3.3-9.4) | Romulania sp. | 3.1 | 9.4 | na | +/+/+ | +/+ | +/+ | + |
| | Pleomassariaceae 2.9-0) | Trematasphaeria pertuso‡ | 2.9 | — | S ⁱ | +/+/− | −/+ | +/+ | − |
| | Pleasporaceae (3.9-14.8) | Alternaria sp. | 3.2 | 14.6 | E ^{c, j} | +/+/+ | +/+ | +/+ | + |
| | Saccharomycetaceae (10.9- | Debaryomyces prosopidis‡ | 10.4 | 31.5 | na | +/+/+ | +/+ | +/+ | + |
| | 31.8) | | | | | | | | |
| | Xylariaceae (0.7-0) | Lopadastama meridionale‡ | 0.3 | — | S ^{k} | −/−/− | −/− | −/− | − |
| | | Lopadastama quercicola‡ | 0.4 | — | S ^{k} | +/−/− | −/− | −/− | − |
| Basidiomycetes | Filobasidiaceae (0.2-0.2) | Filobasidium magnum‡ | 0.1 | 0.2 | na | +/−/− | −/+ | −/− | − |
| (26.7-10.8) | | | | | | | | | |
| | Hymenochaetaceae (15.2-0) | Fomitiporia sp. | 14.6 | — | P ^{c} | +/+/+ | +/+ | +/+ | − |
| | | Fomitipera mediterranea | 0.2 | — | P ^{c} | −/+/− | +/− | −/− | − |
| | | Inonatus hispidus | 0.3 | — | S, P ^{l} | +/−/− | −/+ | −/− | − |
| | Malasseziaceae (0.3-0.3) | Malassezia restricta‡ | 0.2 | 0.2 | na | +/−/− | −/− | −/− | + |
| | Psathyrellaceae (0.5-0) | Psathyrella sp. | 0.5 | — | S ^{m} | +/−/− | −/− | −/− | − |
| | Sporidiobolaceae (0.6-0) | Rhodotorula mucilaginosa† | 0.4 | <0.1 | S ⁿ | −/+/+ | +/+ | −/− | − |
| | Tremellaceae (6.4-8.3) | Cryptococcus sp. | 2.7 | 7.6 | E, S ^{b} | +/+/+ | +/+ | +/+ | + |
| | | Cryptococcus heimaeyensis† | 0.3 | — | — | −/−/+ | −/− | −/− | − |
| | | Cryptococcus victoriae‡ | 2.9 | 0.7 | — | +/+/+ | +/+ | +/+ | + |
| | | TOTAL | 79.1 | 80.8 | | | | | |

* References.
^{a} (Kola\check{r}ík et al, 2017),
^{b} (Jayawardena et al, 2018),
^{c} (Haynes, 2016),
^{d} (Gramaje et al, 2018),
^{e} (González & Tello, 2011),
^{f} (Thambugala etal, 2015),
^{g} (Xing et al, 2011),
^{h} (Casieri et al., 2009),
ⁱ (Suetrong et al., 2011),
^{j} (Pancher et al., 2012),
^{k} (Jaklitsch et al., 2014),
^{l} (González et al., 2009),
^{m} (Bruez et al., 2016),
ⁿ (Ha\check{n}á\check{c}ková et a/., 2017).
‡First report of genus and species in grapevine wood.
†First report of species in grapevine wood.

Objective (1): to characterize the mycobiome of the wood of V. vinifera cv Cabernet Sauvignon, in a vineyard located in the Lisbon area (Portugal), by metabarcoding using Illumina® next-generation sequencing (NGS) techniques;

Objective (2): to understand the spatial distribution of the communities present in different areas of perennial wood and in annual wood.

As mentioned above, among the fungal genera and species that the study found associated with grapevines for the first time, some have previously been detected in the endosphere or phyllosphere of other woody plants that were found in the proximity of the vineyard used in the present study (Table 1). For example, Lopadostoma spp. and Anthostoma gastrinum were reported in Quercus (Jaklitsch et al, 2014; Haynes, 2016), Rhodotorula mucilaginosa and Trematosphaeria pertusa in Fraxinus (Suetrong et al, 2011; Haňáčová et al, 2017), and Malassezia restricta in Eucalyptus (Paulo et al, 2017). This suggests that the fungi present in the endosphere and phyllosphere of the flora in proximity of a vineyard might influence the composition of the mycobiome of grapevines wood, acting as a reservoir of multi-host fungi, with wind, rain and insects being possible vectors for mycobiome exchange.

Many microbes comprising the woody tissues of healthy plants have been found as the causal agents of a number of diseases. Indeed, increasing evidence suggests that under specific conditions, some endophytes may turn into pathogens and lead to development of an infection, paving the way to the subsequent entry of pathogens (e.g. rotting wood microorganisms) and eventually/ultimately causing plant death.

The upsurge observed during the last three decades for some plant wood diseases may well result, at least in part, from an imbalance in the natural wood microbiomes, which may facilitate the entrance and uncontrolled growth of new, invading pathogens or even of microorganisms which are naturally present in healthy wood as endophytes. Several factors, acting alone, in combination or in succession, may contribute overall to plant weakness, thus inducing a microbiome change and facilitating subsequent disease establishment. Moreover, intensive and successive applications of fungicides, as it occurs nowadays in vineyards throughout the world, may lead to residual leakage of systemic fungicides into the heartwood or their simple accumulation in the heartwood as the result of plant growth as the trunk thickens over the years, causing an imbalance in the normal, healthy wood microbiome, which may allow the uncontrolled growth of resistant microorganisms present under these conditions. The knottiness associated to the esca complex of diseases supports this view, as the pathogens involved are often encountered in the wood of healthy plants.

The Use of Microbes and Adaptogens as Antagonists

Fungal and bacterial endophytes are ubiquitous within plants, with the nature of their interactions with the hosts ranging from mutualism, through commensalism, to parasitism. Their number and species composition are influenced by factors such as the host genotype, environment, plant physiology, anthropogenic factors, certainly including pesticide applications, and pathogen infections. Such factors may cause an imbalance in the normal and healthy wood microbiome, thus promoting the uncontrolled development of otherwise harmless microorganisms, much in the same way as Clostridioides difficile (a regular member of the human gut microbiome) boosts are sometimes observed in humans following the ingestion of large spectra antibiotics. Some of these microorganisms may therefore live symptomless in the wood of their hosts for some time in their life and then go from neutral, commensalism or mutualism to parasitism lifestyles. For example, in the case of grapevine, the pathogens known to cause some of the most important trunk diseases (excoriose and esca) have been isolated from inside plant tissues from both symptomatic and asymptomatic plants (Halleen et al, 2007; Gonzalez & Tello, 2011; Núnez-Trujillo et al, 2012; Varanda et al, 2016).

There are now many reports showing that microbial endophytes in general exhibit beneficial effects on their hosts, either acting as plant biostimulants or conferring tolerance to both biotic and abiotic stresses. The role of endophytes in pathogen defense may be achieved by different mechanisms, namely the induction of systemic resistance, accumulation of pathogenesis-related (PR) proteins, expression of plant defense genes, production of secondary metabolites and/or competition in terms of nutrients and space with other microorganisms occupying the same habitat. According to the hypothesis formulated in the present application, an important role played by the healthy wood microbiome may be operating as a buffer, thus preventing the outburst of any species in particular. However, when external factors disturb this natural equilibrium, disease often ensues.

Specific, man-driven changes in the wood microbiome have been used to our benefit, not by attempting restoration of the normal, healthy microbiome, but by using single microbial species as antagonists of the microbial causal agent. Such antagonists are typically first selected by observing an in vitro inhibitory effect on the growth and development of the target microbial pathogen. Thus, for example, Epicoccum species, which rarely infect plants, interfere negatively in vitro with the growth of many microbial pathogens, and E. nigrum has been employed in the management of a large number of plant diseases. Thus, Epicoccum and Alternaria have been shown antagonism against Plasmopara viticola and Botrytis cinerea (Musetti et al, 2007; Polizzotto et al, 2009; Varanda et al, 2016).

Epicoccum naturally found in the wood of olive trees showed some inhibitory action over the growth of Colletotrichum acutatum (Landum et al, 2016). E. nigrum inhibited, both in vitro and in vivo, mycelial growth of Phytophthora infestans, the causal agent of potato late blight (Li et al, 2013). A method to produce E. nigrum conidia in high quantities that could be used in industrial scale in the efficient biocontrol of brown rot of stone fruits has been developed (Larena et al., 2004).

In a similar way to fungal and bacterial endophytes, some plants may have beneficial, neutral or deleterious effects towards soil borne pathogens, such as Phytophthora cinnamomi; the causal agent of cork oak die-back.

Thus, for example, Phlomis purpurea is known to drastically reduce the growth of P. cinnamomi inoculum in the soil by producing metabolites that disrupt formation of the pathogen disease cycle structures. The efficiency of P. purpurea as a suppressive endemic plant may be used as a biocontrol of P. cinnamomi in oak plantations (Neves et al, 2014; Mateus et al, 2016; Baldé et al, 2017).

Techniques Used to Study Microbiomes: Growth on Artificial Culture Media, Metagenomics and Metabarcoding

Any typing technique disclosed herein may be used in the different aspects of the invention. Preferred typing techniques characterise the DNA sequence of an organism, for example by identifying specific markers.

As mentioned above, the concept of microbiome identifies the community of microorganisms (e.g. fungi, bacteria, viruses) that inhabit a particular environment. All environments, ranging from soil to the human body, present a diverse array of microbes that interact among each other and with their hosts. The field of microbial ecology aims to study such diversity and interactions.

The history of microbial ecology underwent important changes from its beginnings to recent days (Morgan et al, 2017). There are three main approaches to the study of the composition of microbiomes, starting from the earliest to the most recent:
(i) The traditional microbiological approach, which characterized the first studies of microbial ecology. Until recently, microbial identification required the isolation of pure cultures. This method is based on the isolation and cultivation of microorganisms in vitro, their identification relying mainly on morphology, microscopy and growth of cultures in different media, as well as testing for multiple physiological and biochemical traits.
(ii) The molecular biology approach, beginning from the polymerase chain reaction (PCR) revolution. This approach is culture-independent and allows the accurate identification of microorganisms through DNA-based fingerprinting. Common methods included in this category are: single strand conformation polymorphism (SSCP), automated ribosomal intergenic spacer analysis (ARISA), denaturing gradient gel electrophoresis (DGGE);
(iii) The next-generation sequencing (NGS) approach, being the results of the latest improvements in DNA sequencing and bioinformatics. Metagenomics consists in the direct analysis of the collective genomes of environmental samples, either in their entirety (whole metagenome sequencing) or focusing on a specific category or organisms (amplicon-based sequencing, also called metabarcoding). In metabarcoding, a specific gene marker (amplicon), from fungi or bacteria, is selected and amplified directly from environmental DNA (eDNA) without any step of enrichment or cultivation (Morgan et al, 2017).

The three approaches described above are all valid and continue to be applied to this day. Nevertheless, culture-dependent and culture-independent approaches offer different understanding. The first methodology is microbe culture-dependent, whereas the other two are culture-independent. The culture-independent approaches are able to identify taxa present in very small abundance and others that are not cultivable in vitro. In addition, with NGS it is possible to describe the diversity of complex environmental samples and with greater resolution (Morgan et al, 2017).

During a very long time, before the advent of molecular biology, it was well established that direct microscopic microbial counts exceed viable-cell counts by several orders of magnitude. Table 4 illustrates this observation for sediment and soil samples. Staley and Konopka (1985) used the expression ‘ ’great plate count anomaly‘ ’ to describe this phenomenon. It is now known that the majority of microscopically visualized cells are viable but do not form visible colonies on artificial growth media. In broad terms, it has been estimated that >99% of microorganisms observable in nature typically do not grow using culture-dependent techniques (Hugenholtz et al, 1998). The introduction of culture-independent techniques revealed that the vast majority of microbial biodiversity had been missed by cultivation-based methods.

TABLE 4
Culturability determined as a percentage of culturable
bacteria in comparison with total cell counts

| Habitat | Culturability (%)^{a} |
|---|---|
| Seawater | 0.001-0.1 |
| Freshwater | 0.25 |
| Mesotrophic lake | 0.1-1 |
| Unpolluted estuarine waters | 0.1-3 |
| Activated sludge | 1-15 |
| Sediments | 0.25 |
| Soil | 0.3 |

^{a}Culturable bacteria are measured as CFU (Amann et al., 1995)

The methodology selected for microbiome analyses in the present application was metabarcoding. In this respect, Cristescu (2014) defined
(i) DNA barcode as a small but specific piece of DNA (marker) which allows distinguishing one species from another. The standardized barcode for most animals is a 658 base pair segment of the mitochondrial cytochrome oxidase I (COI or COX1) gene, the standardized barcode for plants is a fragment of the plastid gene encoding the large subunit of ribulose bisphosphate carboxylase (rbcL) combined with a fragment of the maturase (matA) gene, whereas the barcode for fungi is the nuclear internal transcribed spacer (ITS) of ribosomal DNA.
(ii) DNA barcoding as the identification of species using standardized DNA fragments. Metabarcoding is a rapid method of high-throughput. DNA-based identification of multiple species from a complex and possibly degraded sample of eDNA or from mass collection of specimens. The metabarcoding approach is often applied to microbial communities and is increasingly used for global bio-identification.

Blad

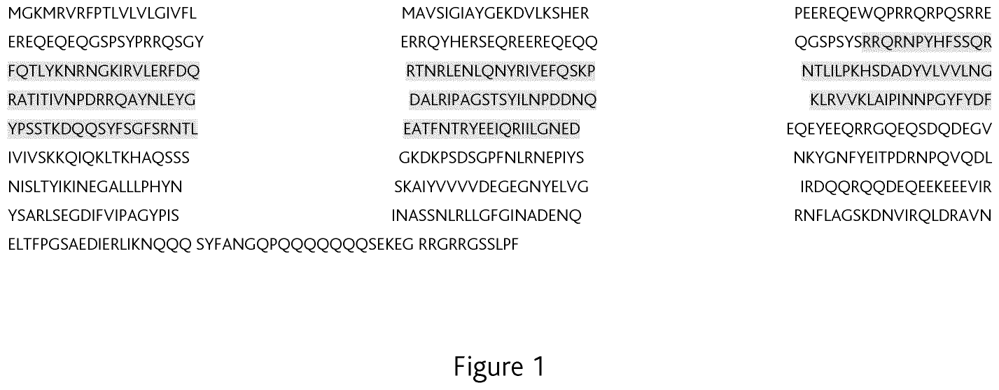

Blad is a 20,408.95 Da, 173 amino-acid-residue-long polypeptide which comprises residues 109 to 281 of the precursor of β-conglutin (i.e. pro-β-conglutin). It is a stable breakdown product, internal fragment of β-conglutin catabolism. β-Conglutin is a globulin, the major storage protein from Lupinus seeds and a vicilin (FIG. 1) (Monteiro et al, 2003, 2006). Under natural conditions, Blad accumulates in the cotyledons of Lupinus seedlings as a subunit of a 210 kDa oligomer (BCO, for Blad-containing oligomer) between the 4^{th} and 14^{th} day after the onset of germination.

Blad Discovery

β-conglutin is synthesized as a precursor (i.e. pro-β-conglutin) during the final stages of one cycle of growth and undergoes very extensive processing (including limited proteolysis and glycosylation) before accumulating in the protein storage vacuoles (PSVs) as mature β-conglutin (Monteiro et al, 2010). During seedling growth in a subsequent cycle of growth, β-conglutin structure and composition undergo a sudden and intense process of limited proteolysis, leading to formation of BCO, which accumulates to high levels, apparently exclusively in the cotyledons of Lupinus seedlings between the 4th and 14th day after the onset of germination. Thereafter, BCO and Blad are degraded to their component amino acids, thus fulfilling their ultimate role as a seed storage protein. Not surprisingly, Blad is particularly rich in nitrogen, with 18 Arg, 17 Asn, 11 Gln and 7 Lys residues, and a total of 266 N atoms. Therefore Blad, the main BCO polypeptide, is a stable breakdown, internal fragment of β-conglutin catabolism (Ramos et al, 1997), and comprises residues 109-281 of Lupinus albus β-conglutin precursor, thus corresponding almost exactly to the first cupin domain of this storage globulin.

Blad Polypeptide

In preferred embodiments, where the antimicrobial agent comprises (or consists essentially of) a polypeptide, said polypeptide comprises (or consists essentially of) Blad or an active variant thereof.

Blad (“banda de Lupinus albus doce”—band from sweet L. albus) was the name given to a stable and intermediary breakdown product of β-conglutin, the major storage protein present in seeds of the Lupinus genus. It was characterised as a 20 kDa polypeptide, composed of 173 amino acid residues, and encoded by an internal fragment (519 nucleotides, deposited in GenBank under the accession number ABB13526) of the gene encoding the precursor of β-conglutin from Lupinus (1791 nucleotides, published in GenBank, under the accession number AA597865). When primers encoding Blad terminal sequences are used to amplify a sequence from genomic Lupinus DNA, a ˜620 bp product is obtained, indicating the presence of an intron in the gene fragment encoding Blad. Naturally-occurring Blad is the main component of a 210 kDa glycooligomer which accumulates exclusively (following intensive limited proteolysis of β-conglutin) in the cotyledons of Lupinus species, between days 4 and 12 after the onset of germination. Whilst said oligomer is glycosylated, naturally-occurring Blad is non-glycosylated. The Blad-containing glycooligomer is composed of several polypeptides, the major ones exhibiting molecular masses of 14, 17, 20, 32, 36, 48 and 50 kDa. The 20 kDa polypeptide, Blad, is by far the most abundant polypeptide within the oligomer and appears to be the only one with lectin activity. Naturally-occurring BCO constitutes approximately 80% of the total cotyledonary protein in 8-day old plantlets.

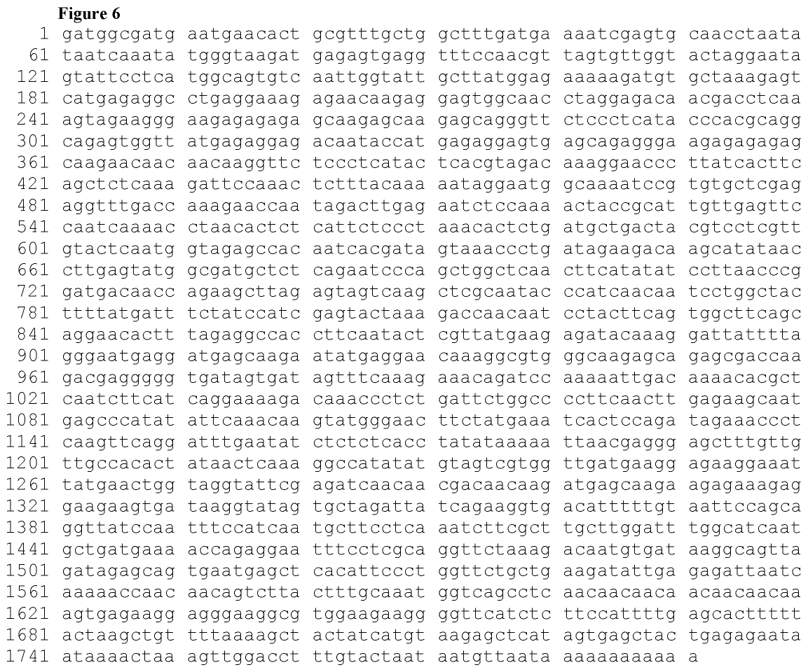

The L. albus β-conglutin precursor encoding sequence (SEQ ID NO: 1) is given in FIG. 6. The β-conglutin parent subunit coding sequence is located at residues 70 to 1668. The encoded, 533 amino acid residue β-conglutin parent subunit (SEQ ID NO: 2) is:

MGKMRVRFPTLVLVLGIVFLMAVSIGIAYGEKDVLKSHERPEEREQEEW
QPRRQRPQSRREEREQEQEQGSPSYPRRQSGYERRQYHERSEQREEREQ
EQQQGSPSYSRRQRNPYHFSSQRFQTLYKNRNGKIRVLERFDQRTNRLE
NLQNYRIVEFQSKPNTLILPKHSDADYVLVVLNGRATITIVNPDRRQAY
NLEYGDALRIPAGSTSYILNPDDNQKLRVVKLAIPINNPGYFYDFYPSS
TKDQQSYFSGFSRNTLEATFNTRYEEIQRIILGNEDEQEYEEQRRGQEQ
SDQDEGVIVIVSKKQIQKLTKHAQSSSGKDKPSDSGPFNLRSNEPIYSN
KYGNFYEITPDRNPQVQDLNISLTYIKINEGALLLPHYNSKAIYVVVVD
EGEGNYELVGIRDQQRQQDEQEEKEEEVIRYSARLSEGDIFVIPAGYPI
SINASSNLRLLGFGINADENQRNFLAGSKDNVIRQLDRAVNELTFPGSA
EDIERLIKNQQQSYFANGQPQQQQQQQSEKEGRRGRRGSSLPF

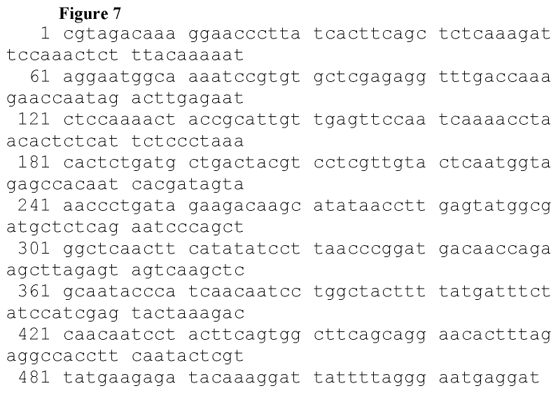

The internal fragment of the β-conglutin precursor encoding sequence that corresponds to Blad (SEQ ID NO: 3) is given in FIG. 7. The Blad polypeptide (SEQ ID NO: 4) is:

RRQRNPYHFSSQRFQTLYKNRNGKIRVLERFDQRTNRLENLQNYRIVEF
QSKPNTLILPKHSDADYVLVVLNGRATITIVNPDRRQAYNLEYGDALRI
PAGSTSYILNPDDNQKLRVVKLAIPINNPGYFYDFYPSSTKDQQSYFSG
FSRNTLEATFNTRYEEIQRIILGNED

Therefore, when the antimicrobial agent comprises (or consists essentially of) a polypeptide comprising (or consists essentially of) Blad or an active variant thereof, said agent comprises (or consists essentially of) a polypeptide sequence comprising (or consisting essentially of) of SEQ ID NO: 4 or an active variant thereof.

An active variant of Blad is a variant of Blad that retains the ability to act as an antimicrobial (i.e. has antimicrobial activity—see below for a description of the level of such activity and how to measure it). “An active variant of Blad” includes within its scope a fragment of SEQ ID NO: 4. In preferred embodiments, a fragment of SEQ ID NO: 4 is selected that is at least 10% of the length of SEQ NO: 4, preferably at least 20%, preferably at least 30%, preferably at least 40%, preferably at least 50%, preferably at least 60%, preferably at least 70%, preferably at least 80%, preferably at least 90% and most preferably at least 95% of the length of SEQ NO: 4. Blad or a variant thereof generally has a length of at least 10 amino acid residues, such as at least 20, 25, 30, 40, 50, 60, 80, 100, 120, 140, 160 or 173 amino acid residues.

“An active variant of Blad” also includes within its scope a polypeptide sequence that has homology with SEQ ID NO: 4, such as at least 40% identity, preferably at least 60%, preferably at least 70%, preferably at least 80%, preferably at least 85%, preferably at least 90%, preferably at least 95%, preferably at least 97%, and most preferably at least 99% identity, for example over the full sequence or over a region of at least 20, preferably at least 30, preferably at least 40, preferably at least 50, preferably at least 60, preferably at least 80, preferably at least 100, preferably at least 120, preferably at least 140, and most preferably at least 160 or more contiguous amino acid residues. Methods of measuring protein homology are well known in the art and it will be understood by those of skill in the art that in the present context, homology is calculated on the basis of amino acid identity (sometimes referred to as “hard homology”).

The homologous active Blad variant typically differs from the polypeptide sequence of SEQ ID NO: 4 by substitution, insertion or deletion, for example from 1, 2, 3, 4, 5 to 8 or more substitutions, deletions or insertions. The substitutions are preferably ‘conservative’, that is to say that an amino acid may be substituted with a similar amino acid, whereby similar amino acids share one of the following groups: aromatic residues (F/H/W/Y), non-polar aliphatic residues (G/A/P/I/L/V), polar-uncharged aliphatics (C/S/T/M/N/Q) and polar-charged aliphatics (D/E/K/R). Preferred sub-groups comprise: G/A/P; I/L/V; C/S/T/M; N/Q; D/E; and K/R.

A polypeptide comprising Blad or an active variant thereof (as described above) may consist of Blad or an active variant thereof with any number of amino acid residues added to the N-terminus and/or the C-terminus provided that the polypeptide retains antimicrobial activity (again, see below for a description of the level of such activity and how to measure it). Preferably, no more than 300 amino acid residues are added to either or both ends of Blad or an active variant thereof, more preferably no more than 200 amino acid residues, preferably no more than 150 amino acid residues, preferably no more than 100 amino acid residues, preferably no more than 80, 60 or 40 amino acid residues, most preferably no more than 20 amino acid residues.

A polypeptide comprising (or consisting essentially of) Blad or an active variant thereof (as described above) may be utilised in the invention in the form of a purified (e.g. removed from a plant, animal or microbial source) or isolated form, and/or may be recombinant. Production of a recombinant form enables the production of active variants of Blad.

Methods of purifying naturally-occurring Blad are already described in the art (e.g. Ramos et al (1997) Planta 203(1): 26-34 and Monteiro et al (2010) PLoS ONE 5(1): e8542). A suitable source of naturally-occurring Blad is a plant of the Lupinus genus, such as Lupinus albus, preferably a cotyledon of said plant, preferably harvested between about 4 to about 14 days after the onset of germination, more preferably harvested 6 to 12 days after the onset of germination (such as 8 days after the onset of germination). Methods are disclosed in the art for a total protein extraction leading to a crude extract comprising Blad, and for a protein purification of such an extract leading to a partially purified extract e.g. comprising the Blad-containing glycooligomer that comprises Blad.

To isolate Blad itself one can then use SDS-PAGE and/or, preferably, reverse phase (RP)-HPLC on a C-18 column.

An alternative way of obtaining a partially purified extract comprising the glycooligomer that comprises Blad is to utilise the chitin binding activity of Blad. The glycooligomer binds in a very strong manner to a chitin column as part of a chitin affinity chromatography purification, being eluted with 0.05 N HCl. Details of an example of this purification method are as follows:

Cotyledons from eight-day old lupin plants were harvested and homogenized in Milli-Q plus water (pH adjusted to 8.0), containing 10 mM CaCl₂ and 10 mM MgCl₂. The homogenate was filtered through cheesecloth and centrifuged at 30,000 g for 1 h at 4° C. The pellet was subsequently suspended in 100 mM Tris-HCl buffer, pH 7.5, containing 10% (w/v) NaCl, 10 mM EDTA and 10 mM EGTA, agitated for 1 h at 4° C., and centrifuged at 30,000 g for 1 h at 4° C. The total globulin fraction, contained in the supernatant, was precipitated with ammonium sulphate (561 g/l), left stirring in the cold for 1 h and centrifuged at 30,000 g for 30 min at 4° C. The pellet obtained was dissolved in 50 mM Tris-HCl buffer, pH 7.5, desalted in PD-10 columns equilibrated in the same buffer and passed through a chitin-affinity chromatography column pre-equilibrated in the same buffer. The column was washed with 50 mM Tris-HCl buffer, 15 pH 7.5, and the bound proteins eluted with 0.05 N HCl. The eluted fractions were immediately neutralized with 2 M Tris and the peak fractions pooled, lyophilized and analyzed by SDS-PAGE.

For the preparation of the chitin column, crude chitin was obtained from Sigma and processed as follows: the chitin sample was washed extensively with Milli-Q plus water, followed by 0.05 N HCl. It was then washed with 1% (w/v) sodium carbonate and then with ethanol, until the absorbance of the wash was less than 0.05. Chitin was then packed into a pipette tip and equilibrated with 50 mM Tris-HCl buffer, pH 7.5.

Methods of producing recombinant proteins are well known in the art. Such methods as applied here will involve inserting the polynucleotide encoding a polypeptide comprising Blad or an active variant thereof into a suitable expression vector—enabling the juxtaposition of said polynucleotide with one or more promoters (e.g. an inducible promoter, such as T7lac) and with other polynucleotides or genes of interest—introducing the expression vector into a suitable cell or organism (e.g. Escherichia coli), expressing the polypeptide in the transformed cell or organism and removing the expressed recombinant polypeptide from that cell or organism. To assist such purification the expression vector may be constructed such that the polynucleotide additionally encodes, for example, a terminal tag that can assist purification: e.g., a tag of histidine residues for affinity purification. Once the recombinant polypeptide is purified, the purification tag may be removed from the polypeptide, e.g., by proteolytic cleavage.

In a composition of the invention that comprises an antimicrobial agent that comprises (or consists essentially of) a polypeptide, said polypeptide is preferably in partially purified form, more preferably in purified form. Said polypeptide is partially purified when it is present in an environment lacking one or more other polypeptides with which it is naturally associated and/or is represented by at least about 10% of the total protein present. Said polypeptide is purified when it is present in an environment lacking all, or most, other polypeptides with which it is naturally associated. For example, purified Blad means that Blad represents at least about 50%, at least about 60%, at least about 70%, at least about 75%, at least about 80%, at least about 85%, at least about 90%, at least about 95%, at least about 97%, at least about 98%, or at least about 99% of the total protein in a composition.

In a composition of the invention that comprises an antimicrobial agent, the Lupinus protein content may consist essentially of the Blad-containing glycooligomer that comprises a polypeptide that comprises (or consist essentially of) Blad or an active variant thereof.

Plant Pathogenic Microorganisms

The plant pathogenic microorganism against which the antimicrobial agent is effective is any microorganism capable of causing disease on or in a plant. Particularly preferred bacterial targets include pathogenic Pseuobmonas species, such as Pseudomonas aeruginosa, Pseudomonas syringae, Pseuobmonas tolaasii and Pseudomonas agaric (preferably P. syringae); pathogenic Erwinia species, such as Erwinia persicina, Pectobacterium carotovorum, Erwinia amylovora, Erwinia chrysanthemi Erwinia psidii and Erwinia tracheiphila, and pathogenic Streptomyces species such as Streptomyces griseus.

Particularly preferred fungal targets include pathogenic Alternaria species, Alternaria arborescens, Alternaria arbusti, Alternaria brassicae, Alternaria brassicicola, Alternaria carotiincultae, Alternaria conjuncta, Alternaria daucd, Alternaria euphorbiicola, Alternaria gaisen, Alternaria infectoria, Alternaria japonica, Alternaria petroselni Alternaria selin, Alternaria solani and Alternaria smyrnii pathogenic Fusarium species, such as Fusarium oxysporum and Fusarium graminearum (preferably F oxysporum); pathogenic Botrytis species, such as Botrytis cinerea; and pathogenic Colletotrichum species, such as Colletotrichum actuatum, Colletotrichum coccodes, Colletotrichum capsicd; Colletotrichum crassipes, Colletotrichum gloeosporioides, Colletotrichum graminicola, Colletotrichum kahawae, Colletotrichum lindemuthianum, Colletotrichum musae, Colletotrichum nigrum, Colletotrichum orbiculare, Colletotrichum pisi and Colletotrichum sublineolum.

Chelating Agents

Blad may be administered with a chelating agent. The chelating agent (also known as a chelant, a chelator or a sequestering agent) is any compound that binds to a metal ion to form a non-covalent complex and reduce the ion's activity. Suitable chelating agents include polyamino carboxylates, such as EDTA (ethylenediaminetetraacetic acid) and EGTA (ethyleneglycol bis(β-aminoethyl ether)-N,N,N′,N′-tetraacetic acid). Preferably, EDTA is used as the chelating agent, preferably at a concentration of at least 10 μg/ml, at least 50 μg/ml, or at least 100 μg/ml, and up to 500 μg/ml, up to 1 mg/ml, up to 5 mg/ml, up to 10 mg/ml, or up to 20 mg/ml. Preferably, EDTA is used at a concentration of between 0.1 mg/ml and 20 mg/ml, more preferably between 1 mg/ml and 20 mg/ml.

Outcomes

The adaptogen may be used to inhibit the growth of a plant pathogenic microorganism (meaning that it has microbistatic activity) and/or to kill said microorganism (meaning that it has microbiocidal activity). The skilled person will be able to identify, through routine methods, a suitable dose and/or concentration of the adaptogen, to obtain a particularly desired growth inhibition or killing of the microorganism.

Preferably, when the adaptogen is used as a microbiostatic, the combination reduces the rate of growth by 10%, more preferably by 50%, more preferably by 75%, more preferably by 90%, more preferably by 95%, more preferably by 98%, more preferably by 99%, and even more preferably by 99.9% in comparison to equivalent conditions where the combination is not present. Most preferably the combination prevents any growth of the microorganism.

Preferably, when adaptogen is used as a microbiocidal, the combination kills 10% of the population of the microorganism, more preferably 50% of said population, more preferably 75% of said population, more preferably 90% of said population, more preferably 95% of said population, more preferably 98% of said population, more preferably 99% of said population, and even more preferably by 99.9% of said population in comparison to equivalent conditions where the combination is not present. Most preferably the combination kills all of the population of the microorganism.

When used to prevent or inhibit infection of a plant by a microorganism the adaptogen is preferably used in an effective amount, that is to say an amount that provides a level of growth inhibition and/or killing of a microorganism such that a detectable level of infection prevention or inhibition is achieved (e.g. a detectable level of prevention or inhibition of plant tissue damage is achieved), preferably in comparison to equivalent conditions where the combination is not present.

Suitable concentrations of Blad include at least 5 μg/ml, at least 10 μg/ml, at least 20 μg/ml, at least 50 μg/ml, at least 100 μg/ml or at least 500 μg/ml, and up to 1 mg/ml, up to 2.5 mg/ml, up to 5 mg/ml or up to 10 mg/ml. Preferably the concentration of said polypeptide is between 50 μg/ml and 10 mg/ml, more preferably between 500 μg/ml and 5 mg/ml, and even more preferably between 1 mg/ml and 5 mg/ml (such as about 2.5 mg/ml).

Uses and Methods

The invention provides the use of a composition of the invention to inhibit the growth of and/or kill a plant pathogenic microorganism on a plant. To this end it also provides a method of inhibiting the growth of and/or killing a plant pathogenic microorganism comprising administering to a plant in need thereof a composition of the invention (e.g. an effective amount of said composition).

The plant in need of the composition of the invention may be any plant that is at risk of acquiring an infection or that has an infection, wherein said infection is caused by a plant pathogenic microorganism. Preferably, the plant in need of the composition of the invention may be a tree (for example a tree in the field of forestry). Preferably, the plant is a woody plant. In preferred embodiments, the plant includes grapevines, olive trees, fruit trees and forest trees. Preferably, the plant is a crop plant (e.g. any plant that is grown to be harvested to provide food, livestock fodder, fuel, fibre, or any other commercially valuable product). Preferably, said crop plant is a food crop plant, such as a plant providing a sugar (e.g. sugar beet, sugar cane), a fruit (including a nut), a vegetable or a seed. Particular plants that provide seeds include cereals (e.g. maize, wheat, barley, sorghum, millet, rice, oats and rye) and legumes (e.g. beans, peas and lentils).

Optionally the adaptogen, including Blad or skopobiota, may be used in isolated form.

Blad Includes Homologues

The variants may be defined with reference to a percentage identity to SEQ ID NO:4. Thus they may be defined using a strict structural definition which limits the variants to those polypeptides which are closely related to SEQ ID NO:4. That definition of the variants completely describes each of them in a full, clear, concise and exact way.

The specification discloses the sequence SEQ ID NO:4 and provides a description of homologues in terms of percentage homology and in terms of the numbers of modifications that may be made to SEQ ID NO:4. In addition the antimicrobial activity of the variants are described in detail and the Examples in previous patents have shown how this may be tested. Thus, from the disclosure in the specification the skilled person could derive all the functional variants defined herein.

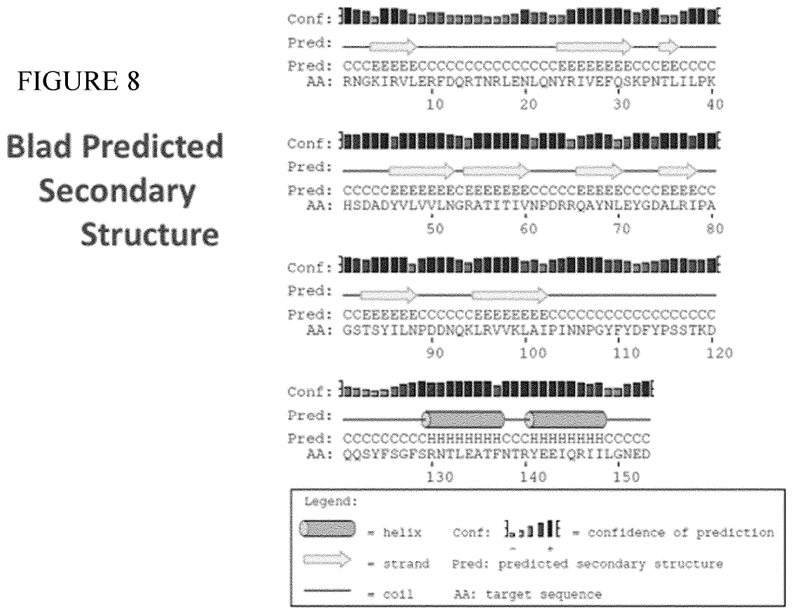

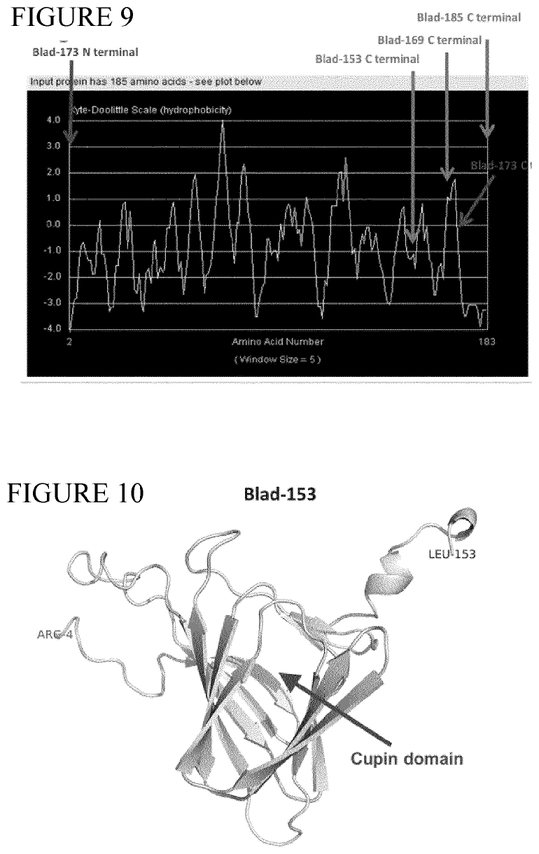

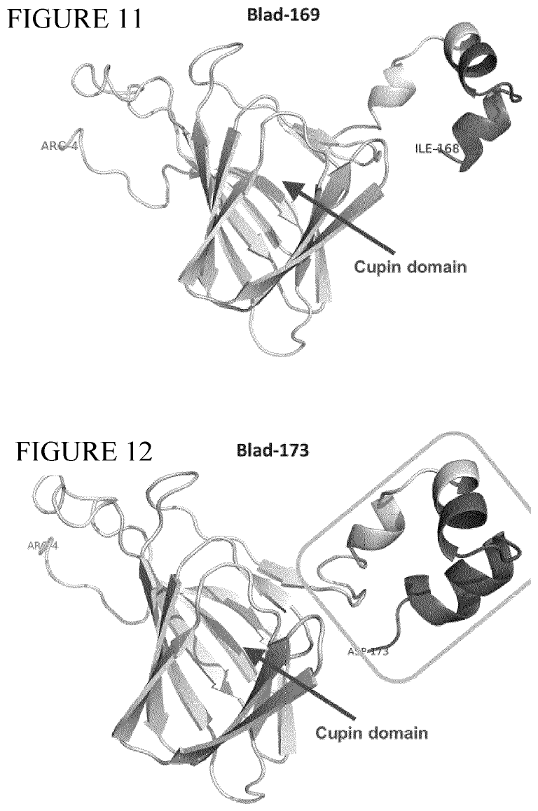

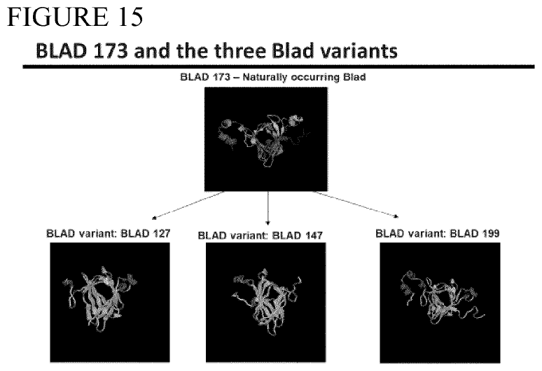

Information about structure can be derived directly from the sequence in cases where the sequence allows this and where the family from which the polypeptide derives has been well characterised. In the present case both of these allow accurate prediction of the structure of the polypeptide and homologous sequences. Attached FIGS. 11 and 12 show the prediction of the secondary structure and a hydrophobicity plot for the sequence. As can be seen from FIG. 11 the structure can be predicted for most of the sequence with a very high confidence. This information allows determination of the three dimensional sequence of SEQ ID NO: 4 which is shown in attached FIG. 15 (SEQ ID NO:4 corresponds to Blad-173).

Further guidance on the structure of the polypeptide can be derived from the fact that the skilled person would recognise from the sequence that it is a member of the cupin superfamily. The well-known cupin domain is shown on FIG. 15 in the centre of the structure. The cupin superfamily proteins are characterised by a conserved β-barrel. Many structures from the cupin proteins are available from public databases. For example the use of the keyword ‘cupin’ produced 128 structure hits in the Protein Data Bank (http://www.rcsb.org/pdb/results/results.do?tabtoshow=Current&qrid=B2756C99) on 3 Dec. 2012.

There may be many functional homologues of SEQ ID NO:4. In the present case given that the skilled person is able to predict the three-dimensional structure of the protein and can also rely on the many structures of cupin proteins which are publically available the applicant submits that the skilled person could identify functional homologues (i.e. active variants) by routine means. For the cupin family of proteins it is known that maintenance of the structure of the cupin domain is required, and this can guide the making of changes within the sequence of the polypeptide.

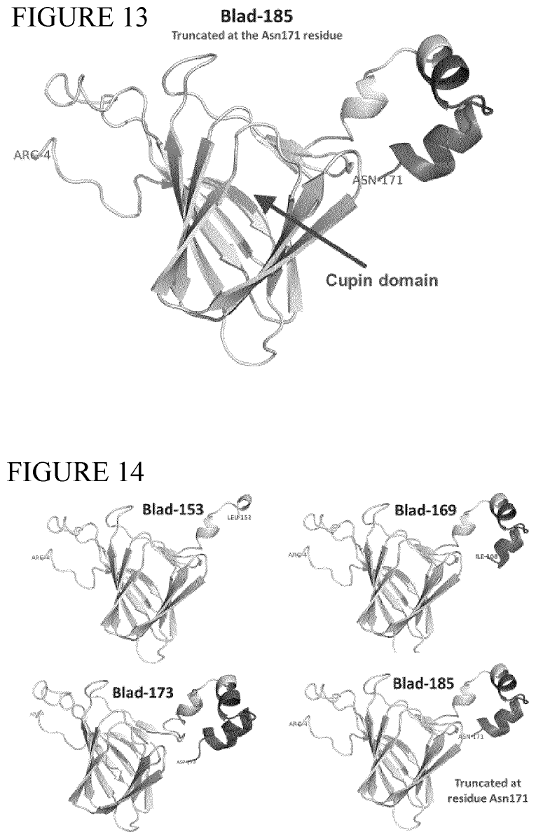

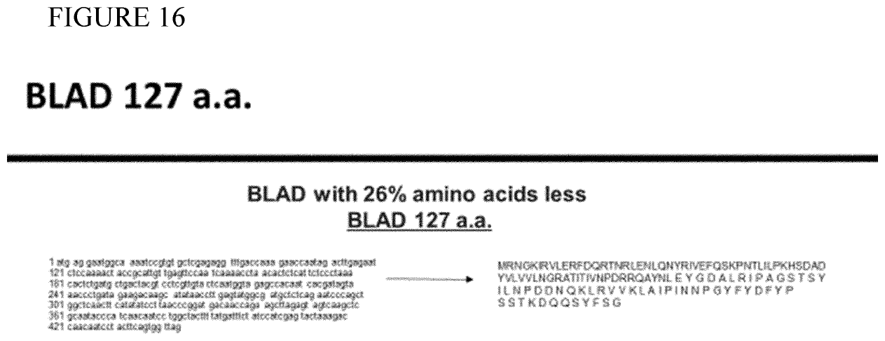

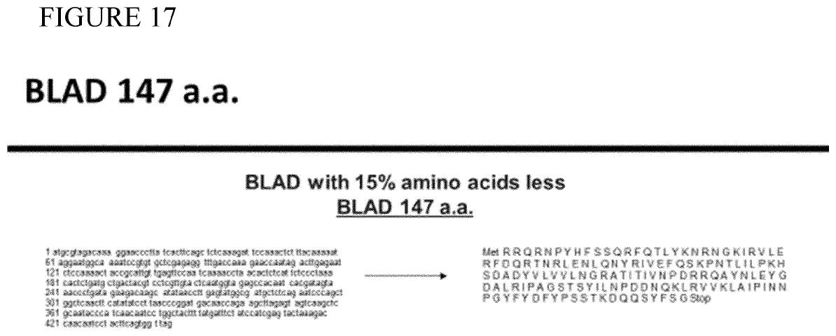

Attached FIGS. 13, 14 and 16 show the predicted structures of variants of SEQ ID NO:4, and these can be compared with SEQ ID NO:4 in FIG. 17. The number in the name of each protein corresponds to its length. Blad-153 and Blad 169 are truncated mutants, and Blad-185 is an extended protein.

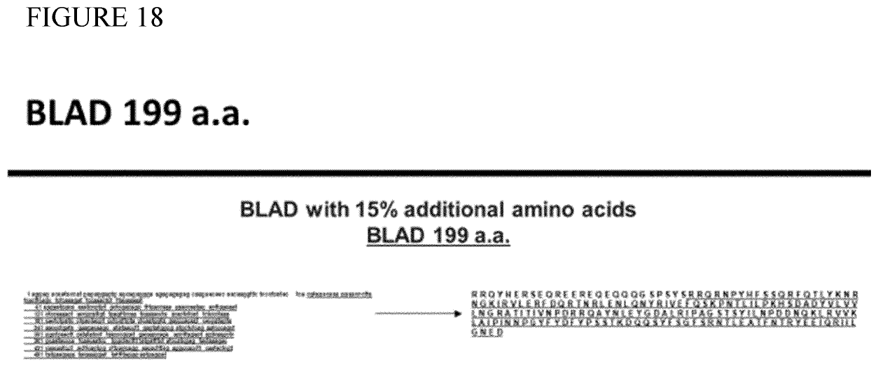

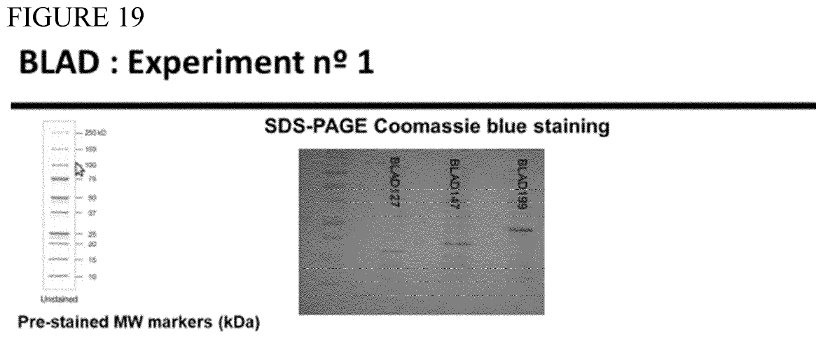

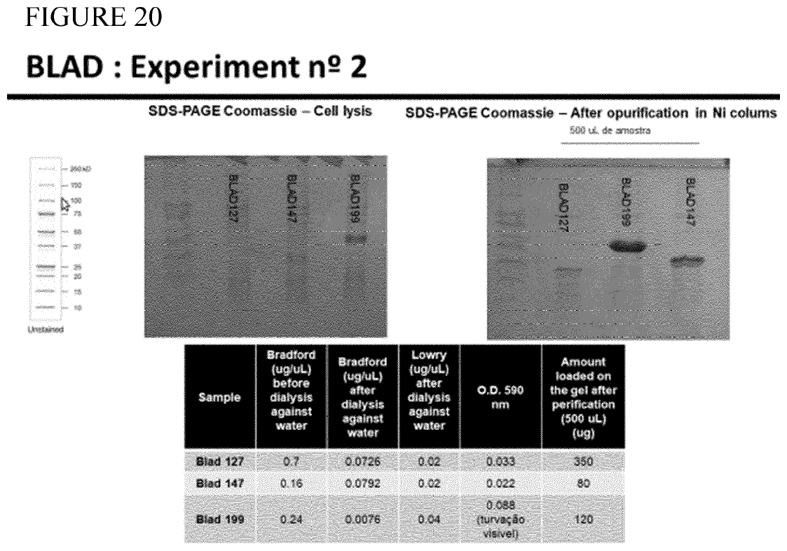

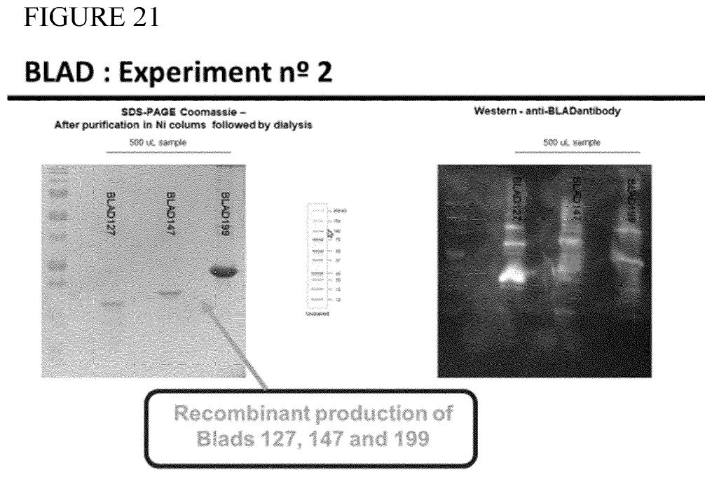

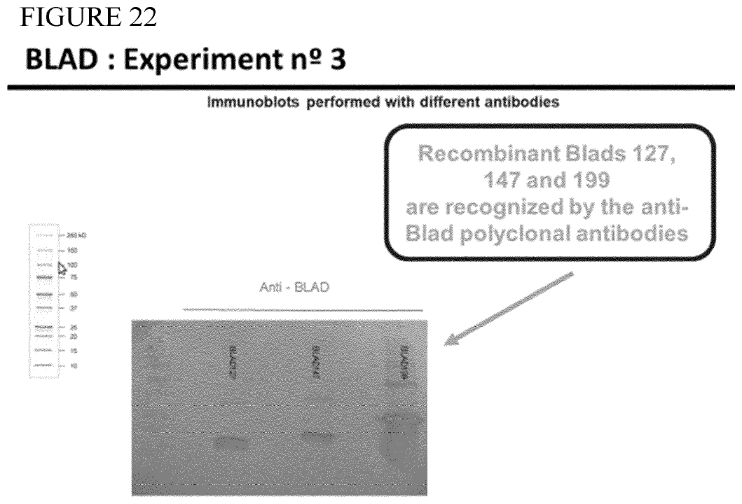

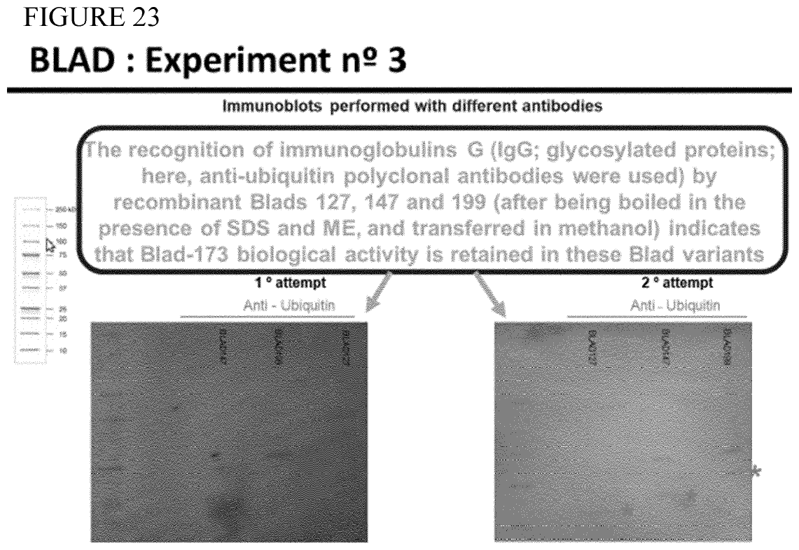

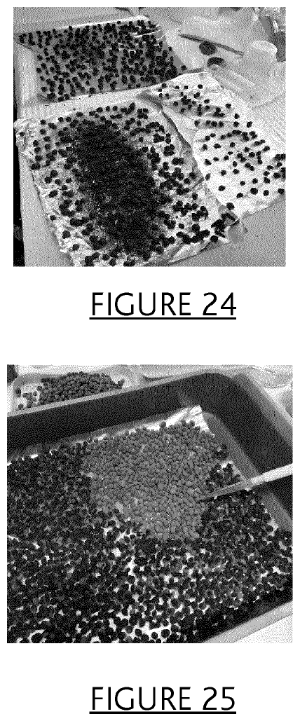

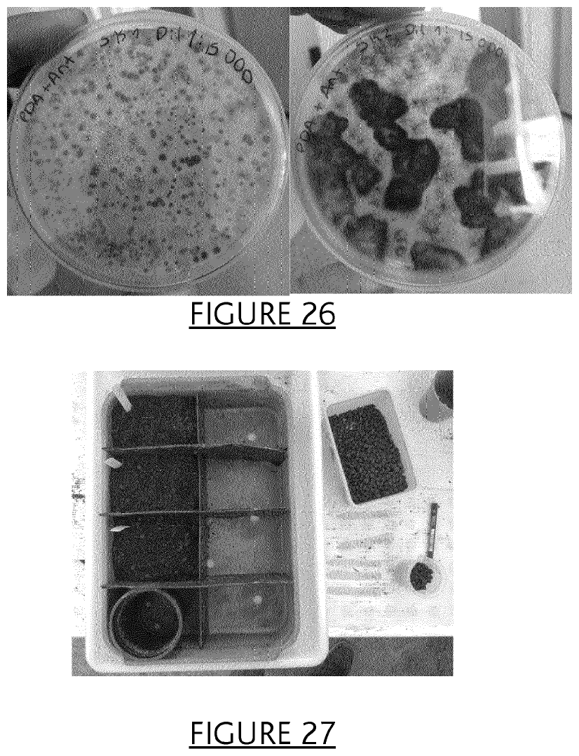

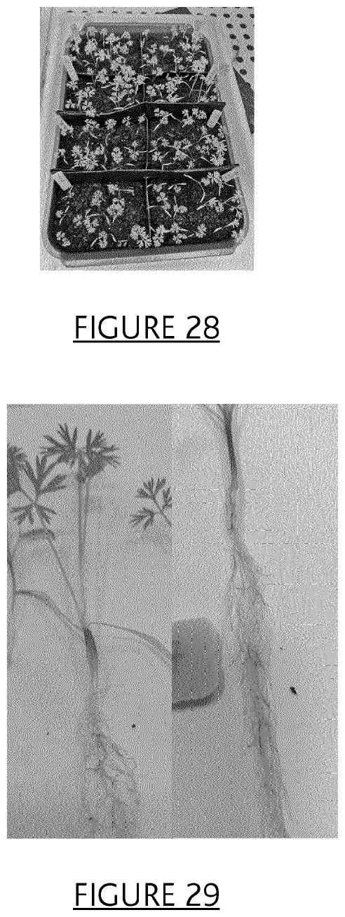

Attached FIGS. 18 to 21 describe further mutants with truncations or an addition. As can be seen the modification to the protein is much more extensive in these cases (with a 26% deletion in one case). Attached FIGS. 22 to 25 show that these three variants (Blad-127, Blad-147 and Blad-199) retain their structure and can be recognised by anti-Blad antibodies that recognise the original protein. Attached FIG. 26 shows that the variant proteins retain the function of binding to a glycosylated protein (in this case a glycosylated IgG protein). This binding activity is being used by the researchers as a marker for functional biological activity. Thus many active variants of SEQ ID NO:4 exist, including some with extensive changes which retain structure and function.

Blad Previously Known Bioactivities

BCO exhibits a potent fungicide activity towards all fungi tested so far, including human, animal and plant pathogens, as well as food spoiling and food poisoning molds and yeasts. Its activity equals or surpasses those of the best fungicides commercially available. In addition, it shows a strong plant biostimulating activity and also bacteriostatic activity towards Gram—bacteria. The antifungal mechanism of Blad has been studied and was demonstrated to be multitarget and rather complex (Monteiro et al, 2015; Pinheiro et al, 2016, 2017). It involves starving the pathogens for certain divalent cations (e.g. zinc, iron and manganese; thus exerting its antifungal activity through inhibition of metal ion homeostasis which results in apoptotic cell death in C. albicans), binding to glycoproteins and to chitin (due to its lectin activity), therefore binding to the fungal cell walls and cell membranes, cleaving terminal residues from chitin (‘exochitinase’, due to its β-N-acetyl-D-glucosaminidase activity), cleaving internal bonds in chitosan (‘endochitosanase’, due to its chitosanase activity), affecting sterol synthesis, entering the cell, causing oxidative stress and ultimately bursting the cells of pathogens. This suggests that the target organisms will probably have a low tendency to develop resistance mechanisms. For these reasons and in what agricultural applications are concerned, in the FRAC Code List© 2018 (available at http://www.phi-base.org/images/fracCodeList.pdf), Fungicides sorted by mode of action, Blad was given the FRAC Code number BM01 (BM meaning ‘Biologicals with multiple modes of action’). We were unable to detect any anti-oxidant, anti-inflammatory and anti-cancer activities for BCO. However, the mechanisms for the biosynthesis of Blad from its precursor (i.e. β-conglutin), Blad bacteriostatic activity towards Gram—bacteria, and its potent plant biostimulating activity remain totally unknown. BCO is certainly a remarkable, edible and non-toxic (in what animals and plants are concerned; it is toxic to the chitin-containing insects but only at very high doses, as it passed through the bee and other insect tests required during the certification procedures) fungicide. It has been certified to be used on US agriculture (by EPA) and, in November 2017, on organic farming by OMRI, Organic Materials Review Institute, 2018, under “Problad Verde (CEV-10083)” OMRI Certificate, available at https://www.omri.org/mfg/cev/certificate/10083.

On the 23 Apr. 2019 issue of the OMRI Products List, available at https://www.omri.org/sites/default/files/opl_pdf/CropByProduct-NOP.pdf, Problad Verde was sorted by company name, page 38, available at https://www.omri.org/sites/default/files/opl.pdf/CompleteCompany-NOP.pdf

Sorted by category, page 63, available at https://www.omri.org/sites/default/files/opl.pdf/CropByCategory-NOP.pdf

Sorted by Product Name, available in page 92 at https://www.omri.org/sites/default/files/opl.pdf/CropByProduct-NOP.pdf

BCO was also certified in a number of other countries and it will be on sale in Europe by 2020.

BCO is a truly remarkable, unique protein with potential to revolutionize the way we currently deal with fungi. Blad is an extremely promising, edible (phyto)pharmaceutical, which exhibits a potent fungicide activity towards human, animal and plant pathogens, bacteriostatic activity and also plant biostimulant activity. A number of articles were published by the applicant about it (e.g. Ramos et al, 1997; Monteiro et al, 2010, 2015; Pinheiro et al, 2016, 2017; Pinheiro et al, 2018; Carreira et al, 2018).

Note: a definition for plant biostimulant has been provided by the European Biostimulants Industry Council (EBIC) (available at http://www.biostimulants.eu/2011/10/biostimulants-definition-agreed/):
“Agricultural biostimulants include diverse formulations of compounds, substances and other products that are applied to plants or soils to regulate and enhance the crop's physiological processes, thus making them more efficient. Biostimulants act on plant physiology through different pathways than nutrients to improve crop vigour, yields, quality and post-harvest shelf life/conservation, but differ from crop protection products because they act only on the plant's vigour and do not have any direct actions against pests or disease. Crop biostimulation is thus complementary to crop nutrition and crop protection.”

Blad/BCO is a contact and non-translocable fungicide, meaning that it is not taken up into the plant tissues and protects only the plant where the spray is deposited.

Blad is a nontoxic fungicide and anti-Oomycete that may be applied in conventional agriculture producing results, based on tests performed by independent companies under real agricultural conditions in many regions of the world, which are equal or better than those obtained with the best commercially available (and toxic) fungicides.

Aspects of the Invention Relating to Skopobiota

The phyllosphere, rhizosphere and endosphere of grapevine (Vitis vinifera L.) are characterized by the presence of complex communities of microorganisms that constantly interact with one another and with the plant, affecting it positively, neutrally or negatively (Bruez et al, 2014). Until a decade ago, the approach to characterize the mycobiome—namely the fungal community present in/on an organism—of grapevines, focused on culture dependent studies in which fungi were isolated in vitro and identified morphologically and/or molecularly (Morgan et al, 2017). This approach remains valid to this day, however it presents several limitations, such as the impossibility of detecting uncultivable fungi, the bias of the cultivation conditions (e.g. growth medium, incubation parameters) and the difficulty of isolating species present in low abundances (Morgan et al, 2017). In recent years, technologies like next-generation sequencing (NGS) have improved in quality and reduced in cost, which, in combination with ever more efficient bioinformatics tools, have allowed the exploitation of this method in the study of the molecular ecology of environmental DNA (eDNA) samples. In particular, DNA metabarcoding approaches have taken the investigations of microbiomes to a new level, surpassing some of the limitations which characterize culture-dependent studies. In fact, NGS studies have revealed a higher diversity of taxa and accurate relative abundances in samples coming from different environments, including the vineyard (Morgan et al., 2017; Jayawardena et al., 2018). Despite these recent advances, culture-independent studies describing the microbial endosphere of grapevines are still scarce.

DNA metabarcoding is a promising tool to investigate the microbial communities present in the wood of grapevines, as it may lead to a new understanding of the complexity that characterizes grapevine trunk diseases (GTDs) and other syndromes which interest the endosphere of woody plants.

X-Ray Tomography

In one embodiment typing and/or diagnosis as described herein comprises X-ray tomography, for example as described in International Application No. PCT/EP2011/068320 the entirety of which is incorporated herein by reference, including specifically any method which is described in that document. Such a method may be used as part of the present invention, for example in combination with a method described herein.

Plant Defense Boosters

A couple of well-established plant defense boosters were used essentially as a control, to allow a direct comparison to the results achieved with Blad. Phosphonate bioactivities in plants were discovered in the 70s. Soon after this discovery, fosetyl-AI was formulated under the trade name Aliette, and released for commercial use (Guest & Grant, 1991). Their salts are believed to exhibit both fungicide and defense booster activities in plants. They were shown to be effective in the control of oomycetes, such as Phytophthora, Plasmopara and Pythium. Many tests have been performed on the effect of phosphonates (and of their potassium salt in particular) on Phytophthora (see for example, Ouimette & Coffey, 1989). Foliar applications of K phosphonate have been successfully used in chestnut trees infected with P. cinnamomi (Coelho, 2009). Unfortunately, the use of K phosphonate to control Phytophthora is far from reasonable. Salicylic acid is a multifaceted bioactive compound. It is translocated within the pant. It is a well-known plant hormone, playing a crucial role in the regulation of physiological and biochemical processes during the entire lifespan of the plant. In addition, is fulfils important roles in the plant responses to both biotic and abiotic stresses (Vicente and Plasencia, 2011). Salicylic acid boosting effects on plant defenses against biotic stresses have been widely documented by exogenous foliar applications. Unfortunately salicylate, like K phosphonate, has not proven good enough to find general application in agriculture/forestry.

Metabarcoding Analysis & Mycobiome Exchange

Grapevine esca and cork oak die-back have been known for many years. However, it was not until the 80s that these diseases became prevalent and started spreading ceaselessly in a way we feel powerless. Nobody knows exactly what the causes are, but most certainly they result from multifactorial processes. Current trade and the generalized movement of goods and people on one hand, and a combination of factors, including but not limited to climate variation, intensive application of pesticides, systematic grafting, wounding induced by pruning and cork removal, reduced precipitation, poor soils, thin soils, lack or excess of organic matter, overgrazing and other changes in agricultural and forest management practices, on the other are among the most favored causes. In addition, the nutritional, health and environmental importance of microbiomes cannot be overstated.

The upsurge observed during the last three decades for some plant wood diseases may well result, at least in part, from an imbalance in the natural wood microbiomes, which may facilitate the entrance and uncontrolled growth of new, invading pathogens or even of microorganisms which are naturally present in healthy wood as endophytes. Several factors, acting alone, in combination or in succession, may contribute overall to plant weakness, thus inducing a microbiome change and facilitating subsequent disease establishment. Intensive and successive applications of fungicides, as it occurs nowadays in vineyards throughout the world, may lead to residual leakage of systemic fungicides into the heartwood, causing an imbalance in the normal, healthy wood microbiome, which may allow the uncontrolled growth of the most resistant microorganisms present under these conditions. The results presented below, as well as some results available in the literature fit well with this hypothesis (Marco et al, 2011). The knottiness associated to the esca complex of diseases also supports this view, as the pathogens involved are often encountered in the wood of healthy plants. Very recent experiments performed in the applicant's laboratory suggest that inoculation of potted vines kept under greenhouse conditions with a specific artificial mycobiome—skopobiota—(composed of four endophytic fungi) may confer a high degree of protection against esca-related pathogen attack. Thus, for example, co-inoculation (in different parts of the same trunk) of Phaeomoniella chlamydospora and an artificial mycobiome (comprising spore suspensions from four fungal species naturally occurring in the grapevine microbiome) reduced the relative abundance of this pathogen to less than 10%. This result was not improved when the plants were sprayed with commercial chemical fungicides. However, the application of Blad (see tool 1) further reduced the relative abundance of P. chlamydospora to 1%.

Metabarcoding Analyses of Woody Tissues

The importance played by microbiomes in natural environments is well-known and has been stated above. It has been known for over a century that the overwhelming majority of microbial species do not grow on synthetic media in vitro and remain unexplored. Uncultured bacteria (i.e. those incapable of growth under laboratory conditions) make up approximately 99% of all species in external environments (Ling et al, 2015). Utilizing the method reported by Nichols et al (2010), Ling et al (2015) achieved a growth recovery approaching 50%, as compared to 1% of cells from soil that grow on a nutrient-containing Petri dish. Grapevine wood (as that of plants in general) is colonized by a wide array of endophytes, microorganisms that reside asymptomatically within interior tissues of living plants for all or part of their life cycle. Typically, they may have advantageous or neutral effects to the plant without causing disease symptoms. However, there is increasing evidence that certain endophytes, such as some of those responsible for esca, may lie dormant for some time in healthy plants and then tum pathogenic in response to one or more unknown stimuli. Many endophytes are uncultivable. For this reason, culture-independent, next generation sequencing (NGS) approaches (e.g. DNA metabarcoding, defined as the combined use of universal DNA barcodes and highthroughput sequencing) are most favorably utilized to characterize biological communities from genetic material collected from environmental samples (Laroche et al, 2017) than culture-dependent morphologically and molecularly (incorporating phylogenetic analysis using multiple genes) identified fungal species that are derived from the same sample. A comparison between culture-independent and culture-dependent procedures for identifying endophytic fungi in stems of grapevine has been recently reported (Dissanayake et al, 2018). Most metabarcoding monitoring studies use DNA to characterize biological communities. A limited number have also evaluated data from co-extracted RNA products (e.g., Pawlowski et al, 2014; Laroche et al, 2017). Because RNA deteriorates rapidly after cell death, RNA likely provides a more accurate representation of viable communities. Blazewicz et al (2013) suggested that the relative concentration of RNA in the environment provides a robust indication of the growth and adaptation potential of microbial communities.

Microbiome Strengthening as a New Paradigm on Plant Health Promotion

Microbial diversity has been largely described as the key for plant and human health. However, how microbial diversity can be enriched, strengthened or fortified in plants remains largely unknown. It has been well recognized that plant microbes may be beneficial or pathogenic (Pseudomonas is a genus of Gram—bacteria that comprises several species, some of which are beneficial for plants—e.g. P. fluorescens- while others are pathogenic—e.g. P. syringae). In addition, they can have a serious impact on the plant microbiome, enhancing or reducing its overall health.

Many diverse formulations of compounds, substances and microorganisms have been and are being developed with a wide array of objectives. They may be administered to plants or soils to improve plant vigor, yield, quality and tolerance of abiotic stresses. Their modes of action are also quite variable, ranging from stimulation of enzyme activities and hormonal effects, to improving soil nutrient availability. Modification of natural microbial communities has also been suggested, although the underlying molecular, cellular and physiological mechanisms remain largely unknown. The possibility of some compounds to act at the level of the whole plant microbiome and/or beyond that of the surrounding microbiome must now be considered.

So, inducing a healthier microbiome or stabilizing the microbiome in plants may be a key to promote not only growth but also to induce a natural resistance to stresses, both abiotic and biotic. In fact, currently, the overall view is that plants and their associated microorganisms form a holobiont. Beyond the plant host, the plant microbiome is interconnected with the ecosystem. Although the plant microbiome is divided into specific compartments linked with specific microorganisms, it is also connected to the surrounding environment and alterations in one component are likely to affect the others as well. The rhizosphere, the phyllosphere and all above-ground organs are connected between them and to the surrounding ecosystem, so it is very logical that influencing the microbiome in one part of the plant can have an impact on the whole holobiont.

Overall it is the beneficial interplay of the host and its microbiomes and the surrounding ecosystem that is responsible for maintaining the health of the holobiont, while diseases are often correlated with microbial dysbiosis (Berg et al., 2017). Microbial diversity is now identified as a key factor in preventing diseases and can be implemented as a biomarker in plant protection strategies. Diseases are characterized by a microbial dysbiosis and a response of specific microbes, which can act as antagonists or synergists towards pathogens. This means that targeted and predictive biocontrol approaches are possible by developing microbiome-based solutions and has brought about a paradigm shift in the applicant's understanding of its role in health and disease and has substantial consequences for biocontrol and health issues in plants.

Aspects of the Invention

Use of any combination of three novel (as far as these diseases are concerned) non-conventional approaches (e.g. Blad/BCO application at a site far away from the infection location, typically inaccessible from a pesticide point of view, mycobiome modulation or intelligent mycobiome design, and X-ray tomography) to better understand and control a specific type of plant diseases/plagues (i.e. those affecting both functional and non-functional xylem, as well as roots) for which there are neither effective treatments nor prospects of developing them. In addition, there are no ways of controlling their ‘silent’ dissemination and spread. These same approaches may as well succeed in controlling xylem affecting ailments caused by organisms as diverse as insects, nematodes, fungi, oomycetes and bacteria, for example.

The Results

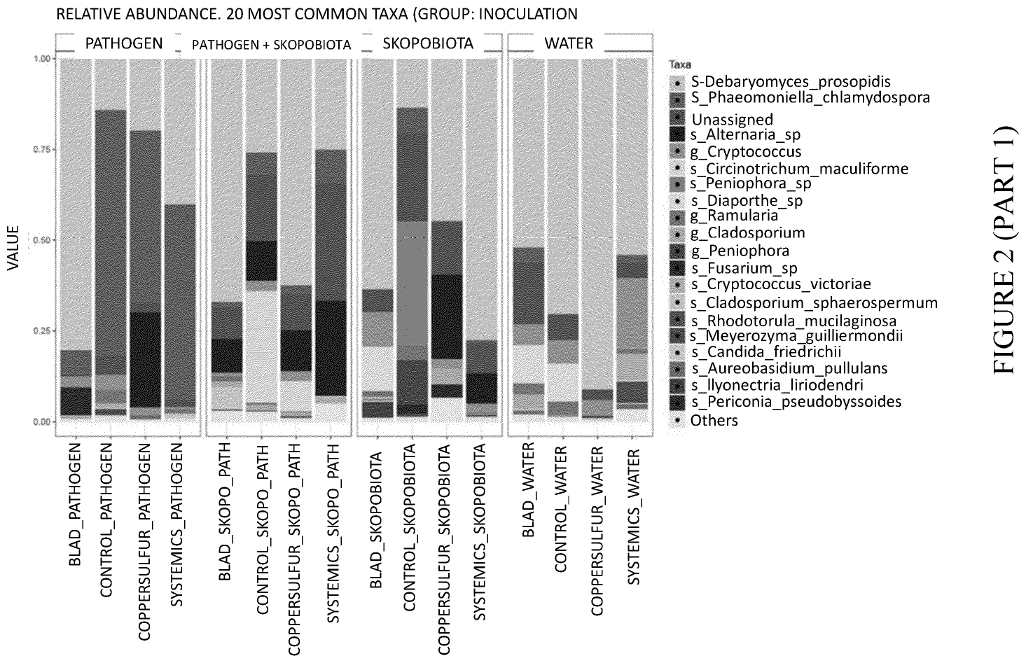

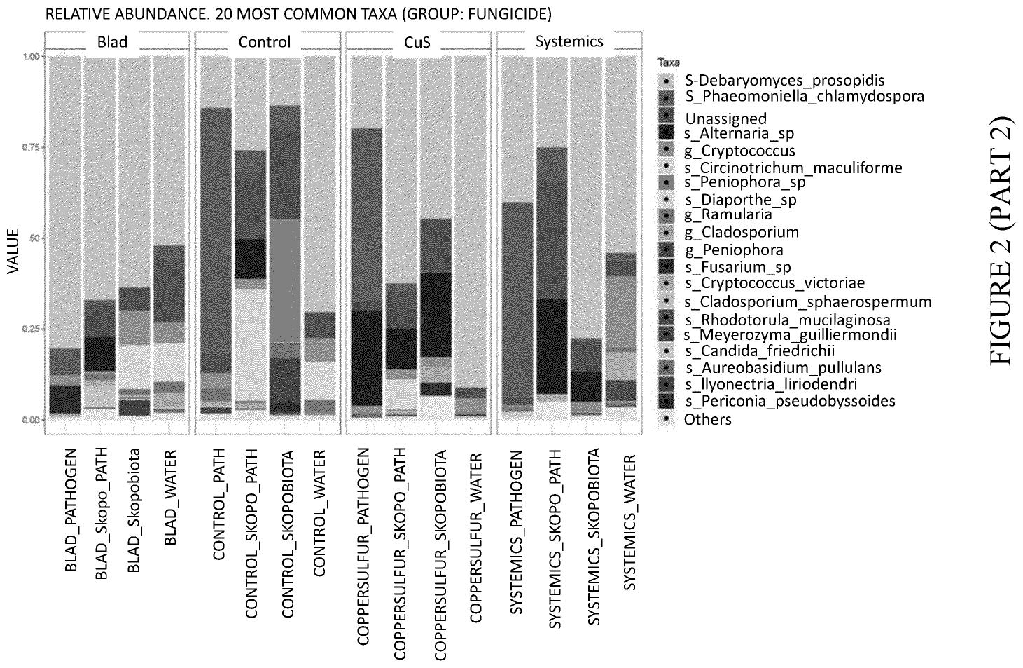

Wood samples were carefully collected from grapevine rooted cuttings of Vitis vinifera cv Cabernet Sauvignon grown under greenhouse conditions, which underwent different inoculations and fungicide treatments and their mycobiomes identified by metabarcoding using Illumina® next-generation sequencing (NGS) techniques. The barplots represented in FIG. 2 show the relative abundances of grapevine wood taxa in plants subjected to different fungicide treatments and inoculation types.

FIG. 2 shows barplots of the relative abundances of the 20 most abundant taxa identified to species (s) or genus (g) level, found in rooted grapevine cuttings non-inoculated (Water), or inoculated with P. chlamydospora(Pathogen) or a consortium of wood endophytes (Skopobiota) or a combination of both (Pathogen+Skopobiota). Grapevines were treated with either Blad-containing oligomer (Blad) or potassium permanganate (Control) or copper oxychloride and sulfur (CuS) or fosetyl-aluminium and penconazol (Systemics). ‘Unassigned’ are taxa identified to a lower taxonomic level than family or non-identified, ‘Others’ are taxa not included in the 20 most abundant. On top, barplots grouped by ‘Inoculation’ type; bottom, barplots grouped by ‘Fungicide’ treatment.

Table 5 shows the relative abundances (%) of P. chlamydospora in potted, rooted grapevine cuttings grown under greenhouse conditions that were non-inoculated (control), inoculated with (P. chlamydospora alone, or inoculated with P. chlamydospora in combination with a skopobiota (P. chlamydospore and skopobiota were administered at separate locations in the trunk), under different fungicide treatments. Note the ‘natural’ presence/infection of Phaeomoniella in some of the control plants, which may indicate that this fungus is present in some plants as an endophyte or, alternatively, that some of the plants, albeit symptomless, may be infected.

TABLE 5

| | | COPPER- | | |
|---|---|---|---|---|
| | CONTROL | SULFUR | SYSTEMICS | BLAD |
| NO-INOCULATION | 0.21 | — | 1.95 | 3.78 |
| P. | 67.62 | 47.24 | 53.4 | 6.31 |
| CHAMYDOSPORA | | | | |
| P. | 5.96 | 2.34 | 9.67 | 1.34 |
| CHLAMYDOSPORA + | | | | |
| SKOPOBIOTA | | | | |

In an experiment designed as an attempt to protect pruning wounds from esca fungal infection, a couple of surprising observations were made when immediately after pruning under real vineyard conditions, the fresh wounds were brushed with 50 μl of water (control), or of Blad, the commercial fungicide Switch or copper oxychloride solutions (24 replicates for each treatment), and 24 h, 1 month and 2 months afterwards the pruning wounds were brushed with P. chlamydospora spore suspensions. Not only the re-isolation rate of P. chlamydospora was very high, but the artificially infected shoots treated with copper presented darker brown streaking when compared with all the other shoots (including the control). On top of that, the diversity of fungi that could be re-isolated in vitro (using culture-dependent methods) from that wood was also lower. In fact, from the shoots treated with Switch and copper, nothing but P. chlamydospora could be re-isolated. These observations suggest that the fungicides killed many fungi that were there but not P. chlamydospora. In all other treatments, such as Blad and the positive control (inoculated only with P. chlamyobspora spores and no fungicide treatment) many other endophytes, such as Alternaria, Botriosphaeria, Epicoccum, Cladosporum, Aureobasidium, and more could be re-isolated. In addition, when testing the fungicides in vitro on cultured media, the Petri dishes which contained potato dextrose agar (PDA) plus individual fungicides like Switch and, to a minor extent, copper, never got contaminated by external fungi. This remarks the efficient fungicide activity of the two chemicals on P. chlamyobspora, although this fungus seems to be less affected than other natural endophytes of the grapevine. The applicant's analyses of the mycobiome by metabarcoding using Illumina® next-generation sequencing (NGS) techniques detected only minor changes in some taxa composition and in colonization success of some endophytes in grapevine wood after a single copper application. The copper/systemic fungicides didn't change the mycobiome composition in major ways and didn't even directly advantage the pathogen.

In the case of esca, for example, one possible trigger of copper may be caused by a differential effect after successive applications of fungicides which may gradually induce imbalances in the wood microbiome, favoring some endophytic microorganisms at the expense of others. Marco & Mugnai (2011) tested a copper-based chemical against P. chlamydospora infections under greenhouse conditions. The study showed that copper was absorbed into the wood of the plants, but P. chlamydospora was not affected (when compared to the other fungi) by its presence (even at concentrations that would normally kill the fungus in vitro). Joining together these observations, it is tempting to speculate the that copper might well be one of the factors responsible for the outbreak of esca-related fungi (or at least P. chlamyobspora) via an imbalance on the grapevine wood microbiome.

This theory, which is quite simple but, at this stage, fairly well motivated, goes like this: Consecutive sprays with copper-based (and other) fungicides over the years may imply not only some copper diffusion to the inner wood regions, but also accumulation in the xylem over the years as the trunk and stems grow thicker. If P. chlamydospora is indeed less sensitive to copper (and/or to some other chemical fungicides), this may result in a gradual microbiome imbalance as the years go by. This could mean ‘weakening’ the wood microbiome of grapevines, by killing or inhibiting the growth of those natural antagonists that in the past kept P. chlamydospora under control. Supposedly, the Applicant may argue that the boom of esca infections began shortly after the introduction of intensive copper spraying. After a lag-phase of maybe 10 to 20 years, esca infections attained a critical level, after which it started spreading exponentially, reaching uncontrollable levels nowadays.

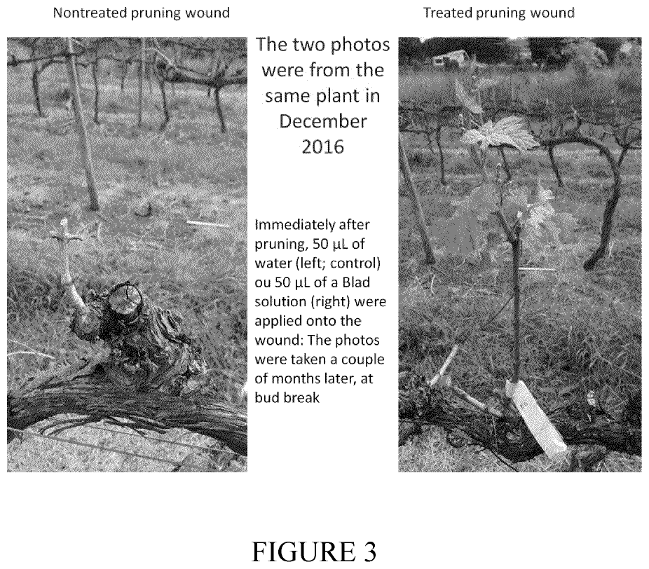

FIG. 3 shows views immediately after pruning, 50 μl of water (Left, control) or 50 μl of a Blad/BCO solution (right) were applied onto the wounds. The photos were taken at the same plant a couple of months later, at bud break. Compare with the natural development state of the vineyard in the background. Twenty-four replicates were prepared for each treatment.

One other surprising result obtained from the experiment whose results are presented in Table 5 was the boost observed in the early growth (FIG. 3) several months after pruning and immediately after adding 50 μl of a Blad/BCO solution onto the pruning wound (only one treatment was performed per plant). It is somewhat hard to believe that this result may be attributable to a simple biostimulant activity.

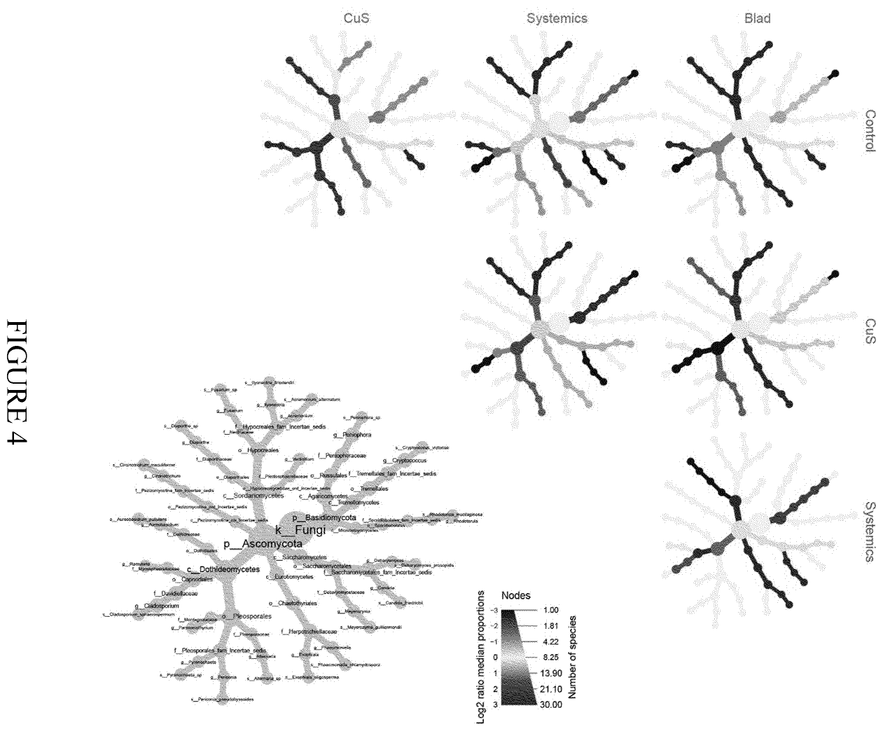

FIG. 4 illustrates the effect of fungicide applications on the grapevine wood relative taxa abundance.

In FIG. 4, the differential heat tree matrix depicts the changes in taxa abundance between different fungicide treatments. The size of the individual nodes in the grey cladogram depicts the number of taxa identified at that taxonomic level. The smaller cladograms show pairwise comparisons between each treatment, with the colour illustrating the log₂ fold change: a red node indicates a lower abundance of the taxon in the tissue group stated on the abscissa, than in the tissue group stated on the ordinate. A blue node indicates the opposite.

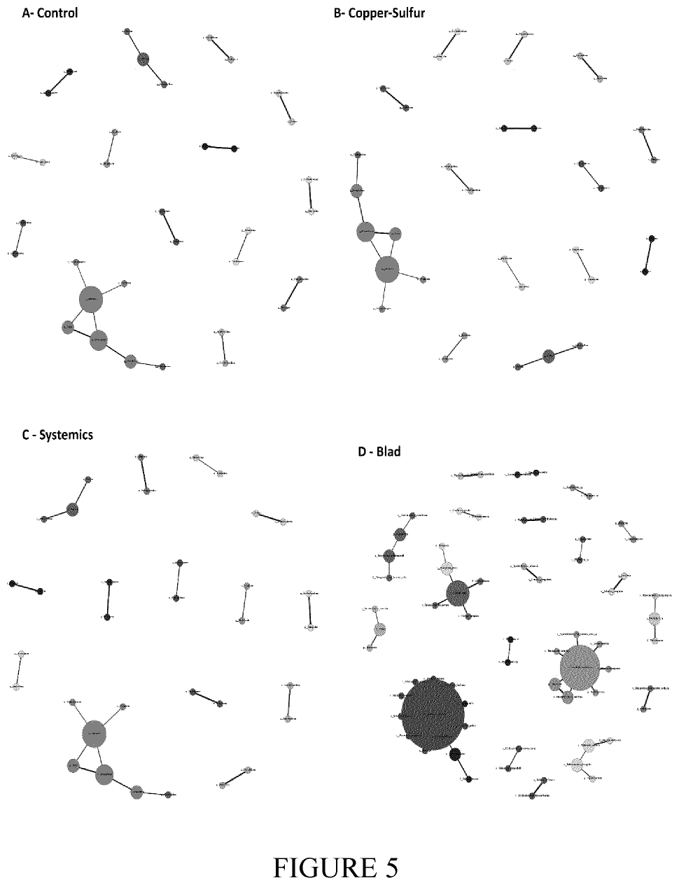

The networks presented in FIG. 5 show how Blad creates a different link among taxa, when compared with control and other fungicides. Control, copper-sulfur and systemic fungicides exhibit similar connections between taxa, whereas Blad, on the other hand, created a different network of interactions in the wood mycobiome.

It is clear from FIGS. 4 and 5 that the foliar application of Blad, unlike the control, copper-sulfur and systemic fungicides, induced marked changes in the grapevine wood mycobiome.

Table 6. List of genera of fungi (endophytes, saprophytes and even pathogens) that may be used to create skopobiotas with the objective of biological control on xylem or root-derived ailments.

Potted vines kept under greenhouse conditions were inoculated with the esca-associated wood pathogen Phaeomoniella chlamydospora and then subjected to the following treatments (relative abundance of the inoculated pathogen):
Positive control: Plants inoculated with P. chlamydospora and not sprayed with fungicides (except for KMnO₄ to control powdery mildew) (67%);
Negative control: Non-inoculated plants (3%);
Plants inoculated with P. chlamydospora and sprayed with copper and sulfur (46%);
Plants inoculated with P. chlamydospora and sprayed with systemic fungicides (54%);
Plants inoculated with P. chlamydospora and sprayed with Blad/BCO (6%).

In a subsequent experiment (see also below), potted vines were co-inoculated with P. chlamydospora and a skopobiota (spore suspensions from four fungal species naturally occurring in the grapevine microbiome). This co-inoculation reduced the incidence of P. chlamydospora infection to less than 10% for copper and sulfur, systemic fungicides and the positive control. However, in Blad/BCO-treated plants, the relative abundance of P. chlamydospora infection decreased to 1%.

The fungi used to produce the skopobiota and consisting in a consortium of grapevine endophytes, were isolated from grapevine wood (cv Cabernet Sauvignon, Almotivo vineyard, Instituto Superior de Agronomia, University of Lisbon, Lisbon, Portugal) of asymptomatic plants and identified as Alternaria alternata A101, Epicoccum nigrum E279, Cladosporium sp. C22 and Aureobasidium pullulans AU86. All fungi were maintained in Petri dishes with vents, on potato dextrose agar medium (Difco™), at 25° C., in the dark.

In summary, inoculation of potted vines kept under greenhouse conditions with a specific mycobiome confers a high degree of protection against esca-related pathogen attack. Thus, for example, co-inoculation of Phaeomoniella chlamydospora and an artificial mycobiome reduced the relative abundance of this pathogen to less than 10%. This result was not improved when the plants were sprayed with fungicides. However, the application of Blad further reduced the relative abundance of P. chlamydospora to 1%.

In a separate experiment, potted young cork oak plants (Quercus suber) were inoculated with P. cinnamomi and incubated under greenhouse conditions. The plants were sprayed with potassium phosphonate, salicylic acid, Blad and other compounds/extracts under study. The severity of the infection was assessed by quantifying the extension of plant root lesions. Blad was the only treatment producing excellent results on the infection levels.

Potassium phosphonate, salicylic acid and Blad treatments all resulted in values of root lesion severities lower than the inoculated control, being effective as they provided a slower disease evolution. BLAD was by far the most promising treatment, since it caused the lowest lesion severity in cork oak roots, and was the only treatment whose results did not differ significantly from the non-inoculated control.

Further Remarks

It seems now well established that (i) the xylem/root affecting infections/infestations in plants are acquiring an unprecedented and permanently increasing economic importance (or shall we call it devastation), as well as the (ii) inexistence of either effective phytopharmaceutical products/procedures or of prospects of developing them to treat/control these recalcitrant plant ailments. In short, assuming pathogenic fungi do not evolve ways of circumventing our extraordinary immune system, the biggest and extremely serious threats which humanity does not seem to have the capacity to overcome are global warming, multidrug resistant bacteria and the xylem/root affecting plant infections/infestations.

Overall, the results presented in this application seem to indicate that:
1. The spray with different chemical fungicides of potted, non-inoculated plants caused minor changes in the wood mycobiome.
2. The foliar spray with Blad/BCO of potted plants inoculated with P. chlamydospora caused a major decrease in the wood colonization success of this fungus. This observation means that the foliar application of the contact and non-translocable Blad/BCO produces a ‘tele’-effect on pathogenic fungi which are present far away (i.e. stem/trunk or roots, typically inaccessible from a pesticide point of view) from the site of application, involving one or more mechanisms which remain fully unknown at present. This seems to be motivated by (i) an interaction between Blad/BCO and the plant, or (ii) Blad/BCO and the xylem/wood mycobiome (which are again far away from each other and typically inaccessible from a pesticide point of view), or (iii) a combination of both.
3. The network analyses suggest that unlike the other treatments (e.g. chemical fungicides), Blad/BCO modulates the xylem/wood mycobiome, possibly making it more resilient to pathogen attacks, supporting point (2) above. Given Blad/BCO properties, this modulation is neither imputable to a direct contact activity between the protein and the mycobiome nor due to Blad plant biostimulant activity (see above for the biostimulant definition).
4. Inoculation of infected plants with a skopobiota strongly reduces the colonization success by the pathogens, most likely because of competitive interactions (use of space/nutrients/chemical war, etc), while not harming the plant—to some extent—also in agreement with point (2) above in what concerns the interaction between skopobiota and resident mycobiome.
5. The combined use of Blad/BCO and skopobiota increases the magnitude of the success against the pathogen. The synergistic combination of both these procedures is novel and may be applied to all plant diseases.
6. The combined use of X-ray based techniques under nursery, greenhouse or field conditions with either Blad/BCO applications, skopobiota inoculation or both. The synergistic combination of these procedures is novel and may be applied to all plant diseases.

It should be taken into account that the concept of skopobiota inoculation is not to ‘restore’ a healthy wood mycobiome—which may be variable due to several biotic and abiotic factors, geographical location, age of the plants, etc., and may ultimately be challenging to standardize, but rather to improve the existing one to make it more successful against pathogens' attacks. An almost ilimited number of distinct skopobiota compositions (see, for example, the different possible combinations, in genera number and identity, not to mention at the species level, listed in Table 6) are possible. Therefore, many of the known endophytes (or even saprophytes) may be used to create a complex skopobiota that can deal with multiple diseases, or even a specific skopobiota which may be effective against a single disease. Hence the long list of fungal genera listed in Table 6.

Finally, the Applicants are introducing Blad as a novel adaptogenic agent for plants that will act as an overall microbiome strengthener, hence helping in prevention and cure. Blad not only promotes growth, but provides the plant with more copying mechanisms against pathogen attacks. This is very unique and revolutionary. Whilst science understanding of the importance of the microbiome in plant health and disease copying is rapidly growing and gaining importance, with people talking about developing microbiome-based solutions as a paradigm shift in the approach to plant health and disease, the Applicant's discovery can introduce a whole new concept of health promotion in plants.

The data obtained indicates that Blad is a plant adaptogen or adaptogenic substance and in this way it may be considered as a prebiotic-like substance, whereas the skopobiota falls clearly under the probiotic concept. They both act at the level of microbiomes and the results of their application are similar, since they seem to complement each other. Hence and not surprisingly, they exhibit synergism when applied to the same plant. The approaches described under the present invention may well constitute one important step into the future of modem agriculture.

In summary and in similarity with humans, a fit microbiome (either by treatment with the prebiotic-like Blad or by inoculation with the probiotic skopobiota) may enhance a plant capacity to face and cope with internal or external stimuli, such as biotic and abiotic stresses. It is well known that both prebiotics and probiotics are associated with better health and reduced disease risk.

Table 6. List of genera of fungi (endophytes, saprophytes and even selected pathogens) that may be used to create skopobiotas with the objective of biological control on xylem or root-derived ailments or any other plant infection.

Absidia Acremonium Acrocalymma Acrospermum Acrostalagmus Actinomucor Agaricus Albifimbria AMfaria Alternaria Amerosporium Ampelomyces Amphisphaeria Angustimassarina Annulohypoxylon Anthostoma Apiotrichum Aplosporella Apodus Arachnomyces Armillaria Arthrinium Arthrobotrys Arthrographis Articulospora Arxiomyces Ascobolus Ascochyta Ascorhizoctonia Aspergillus Asperisporium Athelia Aureobasidium Bactrodesmium Bartalinia Beauveria Bensingtonia Bertia Biatriospora Bionectria Bipolaris Biscogniauxia Bjerkandera Blumeria Boeremia Botryodiplodia Botryosphaeria Botrytis Briosia Buckleyzyma Cadophora Calonectria Caloplaca Calycella Camarosporium Camillea Campylocarpon Candida Capnodium Capronia Catenulostroma Cenococcum Cephalosporium Ceratobasidium Cercospora Chaetomium Chaetothyrium Chalastospora Cheilymenia Chrysosporium Circinotrichum Citeromyces Cladochytrium Cladophialophora Cladosporium Clathrospora Clathrus Claviceps Clitopilus Clonostachys Cochliobolus Colacogloea Colletotrichum Collophorina Coniella Coniocessia Coniochaeta Coniolariella Coniothecium Coniothyrium Cophinforma Coprinellus Coriolopsis Corticium Coryneopsis Corynespora Coryneum Crepidotus Cryptocine Cryptococcus Cryptophaeella Cryptosphaeria Cryptosporella Cryptovalsa Curvularia Cutaneotrichosporon Cyberlindnera Cylindrocarpon Cylindrodadiella Cyphellophora Cystobasidium Cystoflobasidium Cytospora Dacrymyces Dactylellina Dactylonectria Debaryomyces Deconica Dendrophoma Desarmillaria Desmazierella Devriesia Diaporthe Diatrype Diatrypella Dictyosporium Didymella Didymosphaeria Dinemasporium Dioszegia Diplodia Diplodina Discohainesia Discosia Discostroma Doratomyces Dothiorella Elsinoe Emericella Endobasidium Endoconidioma Engyodontium Epicoccum Eriocercosporella Eriosphaeria Erysiphe Erythricium Erythrobasidium Eucasphaeria Eutypa Eutypella Excipula Exidia Exobasidium Exophiala Exosporium Exserohilum Fellomyces Filobasidium Floricola Fomes Fomitiporella Fomitiporia Funneliformis Fusarium Fuscoporia Fusicladium Ganoderma Geomyces Geotrichum Gibellulopsis Glomerella Gloniopsis Glonium Golovinomyces Gonatobotrys Gonatobotryum Graphium Greeneria Grovesinia Guehomyces Gymnascella Gymnopus Gyrothrix Hannaella Hanseniaspora Hansfordia Hapalopilus Helicobasidium Helminthosporium Hendersonia Herpotrichia Heterobasidion Holtermanniella Hormonema Humicola Hyaloceras Hydnum Hymenochaetopsis Hyphoderma Hyphodermella Hyphodontia Hypocrella Hypoderma Hypoxylon Hysterium Hysterobrevium Hysterographium Ilyonectria Inocutis Inonotus IrpexItersonilia Kalmusia Karstenula Kazachstania Kernia Kluyveromyces Knufia Kondoa Kuehneola Kurtzmanomyces Lachancea Lachnella Lachnum Laetiporus Lasiodiplodia Lecanicillium Lecanidion Lecythophora Lentinus Lenzites Leprocaulon Leptodothiorella Leptosphaeria Leptothyrium Leucosporidium Leucostoma Libertella Lopadostoma Lophidium Lophiostoma Lophiotrema L oranitschkia Lycoperdon Macrophoma Macrophomina Macrosporium Magnaporthe Malassezia Marasmius Mariannaea Marssonina Massariella Massarina Meira Meliola Merismodes Metarhizium Metasphaeria Metschnikowia Meyerozyma Microascus Microdiplodia Microdochium Micropera Microthyrium Minimedusa Moeszia Mollisia Monilinia Monochaetia Monochaetinula Monodictys Monographella Mortierella Mrakia Mucor Mycena Mycosphaerella Myrothecium Myxosporium Naganishia Nectria Nemania Neoanthostomella Neodevriesia Neoerysiphe Neofusicoccum Neomassaria Neonectria Neopestalotiopsis Neophysalospora Neoplaconema Neoscytalidium Neurospora Nrospora Nodulisporium Occultifur Oidiodendron Ophiocordyceps Ophiostoma Orbilia Ostreichnion Ostreola Paecilomyces Papiliotrema Papulospora Paraconiothyrium Paraphaeosphaeria Paraphoma Parasola Pareutypella Passalora Patellaria Penicillium Peniophora Peniophorella Penzigomyces Perenniporia Periconia Pestalotiopsis Petriella Phacidiella Phaeoacremonium Phaeococcomyces Phaeomoniella Phaeotheca Phaeotrichoconis Phakopsora Phallus Phanerochaete Phelinidium Phellinus Phialemoniopsis Phialophora Phialosimplex Phlebia Phlebiopsis Phoma Phyllosticta Physcia Phytophthora Pilidium Pionnotes Plagiostoma Plasmopara Pleospora Pleurophoma Pleurostoma Pleurotus Podospora Preussia Psathyrella Pseudallescheria Pseudocamarosporium Pseudocercospora Pseudogymnoascus Pseudolachnea Pseudopestalotiopsis Pseudopezicula Pseudotaeniolina Pseudozyma Psiloglonium Punctulariopsis Pyrenochaeta Pyrenophora Pyrgemmula Pythium Ramicandelaber Ramichloridium Ramularia Rhabdospora Rhinocladiella Rhizoctonia Rhizomucor Rhizopus Rhodosporidium Rhodotorula Robillarda Roesleria Rosellinia Saccharomyces Sakaguchia Sarcoporia Sarocladium Scheffersomyces Schizophyllum Schizothyrium Sclerostagonospora Sclerotinia Sderotium Scolicotrichum Scopulariopsis Scopuloides Scytalidium Scytinostroma Seimatosporium Seiridium Selenophoma Septoria Septoriella Setophaeosphaeria Simplicillium Sistotremastrum Solicoccozyma Sordaria Spencermartinsia Sphaeropsis Spiromastix Spiromyces Sporidiobolus Sporobolomyces Sporocadus Sporoschisma Stachybotrys Stagonospora Stemphylium Stereum Sterigmatomyces Stigmina Strickeria Stromatoneurospora Talaromyces Taphrina Terana Tetracladium Tetracoccosporium Thanatephorus Thaxteriella Thelonectria Thielavia Tilletiopsis Tomentella Torula Torulaspora Toxicocladosporium Trametes Trematosphaeria Trichaptum Trichocladium Trichoderma Trichosporon Trichothecium Trullula Truncatella Tubaria Typhula Ulocladium Umbelopsis Uncispora Valsaria Veronaea Verpa Verrucocladosporium Verticillium Vishniacozyma Volutella Wallemia Xanthoria Xeromyces Xerotus Xylaria Xylodon Yamadazyma Zetiasplozna Zymoseptoria

In further embodiments in accordance with any of the inventive aspects described herein, plant microbiotas may include in addition to any previously mentioned microbiotas not only endomicrobiotas but those living on the external surface of plant organs in particular in the rhizosphere and phyllosphere and/or living in the soil. In preferred embodiments relating to delivery via the soil, the method further comprises the addition of a specific and precise consortium of microbes with a precisely defined composition suitable to fulfill a specific aim or function (i.e. a skopobiota), such that delivery takes place, preferably, in a uniform way throughout the soil (as the delivery material may be mixed with the soil) during an extended period of time (as the microbes comprising the skopobioata will be gradually released from the solid, porous material employed). The concept of skopobiota may in certain embodiments involve any possible fungal species that is to be included in a microbial consortium specifically and accurately designed for a precise purpose. Embodiments may therefore envisage the use of one or more of the following fungal genera.

Allodus, Allomyces, Allosoma, Aloysiella, Alphitomyces, Alternaria, Alveolaria, Alysisporium, Amallospora, Amanita, Amanitella, Amanitopsis, Amastyis, Amastigosporium, Amaurascus, Amazonia, Amblyosporiopsis, Amblyosporium, Ameghiniella, Ameris, Amerodothis, Amerosporiella, Amerosporis, Amerosporium, Anierostege, Amoebochytrium, Amorphomyces, Amphichaeta, Amphichaete, Amphichaetella, Amphiciliella, Amphicytostroma, Amphididymella, Amphiemia, Amphinectria, Amphischizonia, Amphisphaeria, Amphorula, Ampullaria, Amylirosa, Amylis, Anaphysmene, Anaptychia, Anapyrenium, Anariste, Anatexis, Ancylistaceae, Ancylistes, Andreaea, Andreaeana, Anellaria, Anema, Angatia, Angelinia, Angiopoma, Angiopomopsis, Anhellia, Anisochora, Anisogramma, Anisomjces, Anisomyxa, Anisostomula, Anixia, Anixiopsis, Annularia, Anomomyces, Anomorpha, Anomothallus, Antenella, Antenellina, Antennulariela, Anthina, Anthomyces, Anthomyces, Anthomycetella, Anthostoma, Anthostomaria, Anthostomella, Anthostomellina, Anthracoderma, Anthracoidea, Anthracophyllum, Anthracothecium, Anthurus, Antromyces, Antromycopsis, Anzia, Aorate, Aphanascus, Aphanomyces, Aphanomycopsis, Aphanopeltis, Aphanostigme, Aphysa, Apiocarpella, Apiocrea, Apiognomonia, Apioporthe, Apioporthella, Apiorhynchostoma, Apiosphaeria, Apiospora, Apiosporella, Apiosporina, Apiosporina, Apiosporium, Apiosporopsis, Apiotrabutia, Apiotypa, Aplacodina, Aplanes, Aplopsora, Apocytospora, Apodachlya, Apodya, Aponectria, Aporhytisma, Aporophallus, Aposphaeria, Aposphaeriella, Aposphaeriopsis, Aposporella, Apostemidium, Appendicularia, Apyrenium, Arachniopsis, Arachniotus, Arachnium, Arachnomyces, Arachnopeziza, Araeospora, Araneomyces, Arcangelia, Arcangeliella, Arctomia, Arenaea, Areolaria, Argomycetella, Argopsis, Argynna, Armatela, Armillaria, Amaudiella, Arrhenia, Arrhytldia, Arthonia, Arthoniactis, Arthoniae, Arthoniopsis, Arthotheliopsis, Arthothelium, Arthrinium, Arthrobotryella, Arthrobotrys, Arthrobotryum, Artlirobotryum, Arthropyrenia, Arthropyreniella, Arthrorhynchus, Arthrosporium, Articularia, Articulariella, Articulis, Asbolisia, Aschersonia, Aschersoniopsis, Ascobolaceae, Ascobolae, Ascobolus, Ascocalathium, Ascochyta, Ascochytella, Ascochytopsis, Ascochytula, Ascochytulina, Ascocorticium, Ascodesmis, Ascoidea, Ascoideaceae, Ascomycetela, Ascomycetes, Ascophanae, Ascophanus, Ascopolyporus, Ascosorus, Ascospora, Ascostratum, Ascotricha, Aseroe, Ashbia, Aspergillae, Aspergillopsis, Aspergillus, Aspergillus, Asperisporium, Aspidopyrenis, Aspidopyrenium, Aspidothea, Aspidothelium, Asporomyces, Asterella, Asteridiela, Asteridiellina, Asteridium, Asterina, Asterineae, Asterinella, Asteristium, Asterocalyx, Asteroconium, Asterodon, Asterodothis, Asterolibertia, Asteroma, Asteromassaria, Asteromela, Asteromidium, Asteromyxa, Asteronaevia, Asteronia, Asteropeltis, Asterophyctis, Asterophora, Asteroporum, Asteropsis, Asterosporium, Asterostomella, Asterostomula, Asterostroma, Asterostromela, Asterothyrium, Asterothyrium, Astraeus, Astrocystis, Astrodochium, Astrosphaeriella, Astrotheliae, Astrothelium, Atichia, Atopospora, Atractiela, Atractilina, Atractina, Atractium, Atrichophytum, Auerswaldia, Auerswaldiella, Auerswaldiopsis, Aulacostroma, Aulaxina, Aulographella, Aulographis, Aulographum, Aureobasidium, Aureobasis, Auricularia, Auriculariaceae, Auriculariclla, Autoecomyces Avettaea, Bacidia, Bactrexcipula, Bactridiopsis, Bactridium, Bactrosphaeria, Bactrospora, Baculospora, Baeodromus, Baeomyces, Baeumleria, Baggea, Bagnisiella, Bagnisiopsis, Bakeromyces, Bakerophoma, Balansia, Balansiella, Balansina, Balansiopsis, Balladyna, Balladynella, Balladynopsis, Balsamia, Balzania, Barclayella, Bargellinia, Barlaea, Barlaeina, Barssia, Bartalinia, Barya, Basiascella, Basiascum, Basidiella, Basidiobolus, Basdiobotrys, Basidiomycetes, Basidiophora, Basilocula., Basisporium, Battarina, Battarrea, Battarreopsis, Baunianniella, Baumiella, Beauveria, Beccariella, Beelia, Belonia, Belonidium, Beloniella. Belonioscypha, Belonioscyphella, Belonium, Bclonopeziza, Belonopsis, Belospora, Beltrania, Benguetia, Beniowskia, Berkelella, Berlesiella, Bertia, Bertiella, Bertiella, Biatora, Biatorella, Biatorellina, Biatorina, Bifusella, Bionectria, Bioporthe, Bioscypha, Biotyle, Bispora, Bisporella, Bivonella, Bizzozeria, Bizzozeriella, Blakeslea, Blasdalea, Blastenia, Blastocladia, Blastocladiaceae, Blastodendrum, Blastoderma, Blastodesmia, Blastomyces, Blastomycoides Blastospora, Blastotrichum, Blennoria, Blennoriopsis, Blepharospora, Blodgettia, Bloxamia, Blumenavia, Blytridium, Bodinia, Boerlagella, Bolacotricha, Bolbitius, Boletinus, Boletogaster, Boletopsis, Boletus, Bolinia, Bolosphaera, Bombardia, Bombardiastrum, Bombardiella, Bombyliospora, Bommerella, Bonanseia, Bonia, Bonordeniella, Bonplandiella, Borenquenia, Bostrichonema, Bothrodiscus, Botrydiplis, Botryella, Botryochora, Botryoconis, Botryogene, Botryophoma, Botryorhiza, Botryosphaeria, Botryosphaerostroma, Botryosporium, Botryostroma, Botryotrichum, Botrysphaens, Botrytidae, Botrytis, Bottaria, Boudiera, Boudierella, Bourdotia, Bovilla, Bovista, Bovistella, Bovistoides, Boydia, Brachyascus, Brachysporium, Brefeldiella, Bremia, Bremiella, Brencklea, Brenesiella, Bresadolella, Bresadolia, Bresadolina, Brevilegnia, Briardia, Briarea, Brigantiella, Briosia, Broomeia, Broomella, Brunchorstia, Bryophagus, Bryopogon, Bubakia, Buellia, Bulbothamnidium, Bulgaria, Bulgariaceae, Bulgariastrum, Bulgarella, Bulgariopsis, Bullaria, Bullera, Bulliardella, Burkardia, Burrillia, Butleria, Byssocallis, Byssochlamys, Byssocystis, Byssogene, Byssolecania, Byssoloma, Byssolomae, Byssolophis, Byssonectria, Byssotheciella, Cacosphaeria, Cadophora, Caenomyces Caenothyrium, Caeoma, Calathiscus, Calcarisporium, Caldariomyces, Caldesia, Caldesiella, Calenia, Caleniae, Caliciaceae, Caliciopsis, Calicium, Calidion, Calliospora, Calloria, Calloriella, Calloriopsis, Calocera, Calocladia, Caloderma, Calogloeum, Calolepis Calonectria, Calopactis, Calopeltis, Calopeziza, Calopeziza, Caloplaca, Calosphaeria. Calospora, Calosporella, Calostilbe. Calostilbella, Calostoma, Calothyriella, Calothyriolum, Calothyriopeltis, Calothyriopsis, Calothyris, Calothyriuni Calotrichopsis, Calvatia, Calycela, Calycellina, Calycidium, Calyculosphaeria, Calyptospora, Calyptra, Calyptralegnia, Calyptronectria, Camarographium, Camarops, Camarosporelum, Camarosporium, Camarosporulum, Camarotella, Camillea, Cainpanella, Campbellia, Campoa, Campsotrichum, Camptomeris, Camptomyces, Camptosphaeria, Camptoum, Campylothelium, Candelaria, Candelariella, Candelospora, Candida, Cantharellus, Cantharomyces, Cantharosphaeria, Capillaria, Capnites, Capnodaria, Capnodiaceae, Capnodiastrum, CapnodWlea, Capnodina, Capnodinula, Capnodiopsis, Capnodium, Capnophaeum, Capnostysanus, Capronia, Carestiella, Carlia, Carlosia, Carothecis, Carpenteles, Caryospora, Casaresia, Castagnela, Castoreum, Catabotrys, Catacauma, Catacaumela, Catastoma, Catathelasma, Catenaria, Catenularia, Catharinia, Catilla, Catillaria, Catinaria, Catinella, Catinula, Catocarpus, Caudella, Caudospora, Caudosporella, Cauloglossum Causalis, Celidium, Celtidea, Cenangella, Cenangina, Cenangiopsis, Ctfnangium, Cenococcum, Cephaliophora, Cephalodochium, Cephalomyces, Cephalosporiae, Cephalosporium, Cephalotelium, Cephalotheca, Cephalothecium, Cephalotrichum, Ccracea, Ceraeomyces, Cerastomis, Ceratocarpia, Ceratochaete, Ceratochaetopsis, Ceratocladium, Ceratomyces, Ceratomycetaceae, Ceratophoma, Ceratophorum, Ceratoporthe, Ceratopycnidium, Ceratopycnis, Ceratopycnium, Ceratosperma, Ceratosphaeria, Ceratosporella, Ceratosporium, Ceratostoma, Ceratostomela, Cercidospora, Cercoseptoria, Cercosphaerella, Cercospora, Cercosporella, Cercosporidium, Cercosporina, Cercosporiopsis, Cerebella, Cerillum, Ceriomyces, Cerion, Ceriophora, Ceriospora, Ceriosporella, Cerocorticium, Cerotelium, Cesatiella, Cetraria, Ceuthocarpum, Ceuthodiplospora, Ceuthosira, Ceuthospora, Ceuthosporella, Chaconia, Chaenoderma, Chaenotheca, Chaetalysis, Chaetasbolisia, Chaetaspis, Chaetasterina, Chaetobasidiella, Chaetobasis, Chaetobotrys, Chaetoccratostoma, Chaetoceris, Chaetocladiae, Chaetocladium, Chaetoconidium, Chaetoconis, Chaetocrea, Chaetocytostroma, Chaetodiplis, Chaetodiplodia, Chaetodiplodina, Chaetodiscula, Chaetolentomita, Chaetomastia, Chaetomella, Chaetomeris, Chaetomidium, Chaetomium, Chaetomyces, Chaetopcltiopsis, Chaetopeltis, Chaetopeltopsis, Chaetophiophoma, Chaetophoma, Chaetophomella, Chaetoplaca, Chaetoplea, Chaetopsis, Chaetopyrena, Chaetopyrenis, Chaetosclerophonia, Chaetoscypha, Chaetosira, Chaetospermum, Chaetosphaeria, Chaetosphaeronema, Chaetosphaeropsis, Chaetosticta, Chaetostigme, Chaetostigmella, Chaetostroma, Chaetostroma, Chaetostromella, Chaetostylum, Chaetotheca, Chaetothyrina, Chaetothyriolum, Chaetothyriopsis Chaetothyrium, Chaetotrichum, Chaetozythia, Chaetyllis, Chalara, Chalaropsis, Chalcosphaeria, Chamonixia, Chantransiopsis, Charcotia, Charonectria, Charrinia, Cheilaria, Cheilymenia, Chelisporium, Chevaliera, Chevalieropsis, Chiajea, Chiastospora, Chiloella, Chilomyces, Chilonectria, Chiodectae, Chiodectum, Chiroconium, Chiromycella, Chiromyces, Chiropodium, Chitonia, Chitoniella, Chitonomyces, Chitonospora, Chlamydaleurosporia, Chlamydomucor, Chlamydomyces, Chlamydopus Chlamydosporium, Chloridium, Chlorocaulum, Chlorodothis, Chloropeltis, Chlorophylum, Chlorospleniella, Chlorosplenium, Chlorospora, Chnoopsora, Choanophora, Choanophorae, Choeromyces, Chondrogaster, Chondropodela, Chondropodium, Choriactis, Chorostate, Chorostella, Chroinocrea, Chromocreopsis, Chromocytospora, Chromosporium, Chromotorula, Chrysella, Chrysocelis, Chrysocyclus, Chrysomyces, Chrysomyxa, Chrysopsora, Chrysothrix, Chrysotrichaceae, Chytridiaceae, Chytridiae, Chytridiales, Chytridium, Ciboria, CicadomyceSi Cicinnobela, Cicinnobolus, Cidaris, Ciferria, Ciliaria, Ciliciocarpus Ciliciopodiuin, Ciliciopus, Ciliella, Ciliochora, Ciliofusa, Ciiiofusarium, Ciliomyces, Ciliophora, Ciliospora, Ciliosporella. Cintractia, Cionothrix, Circinastrun; Circinella, Circinotrichum, Ciromyces, Cirsosia, Cirsosiella, Citromyccs, Cladobotryum, Cladochaete, Cladochytnae, Cladochytrium, Cladoderris Cladographium, Cladonia, Cladoniaceae, Cladorhinum, Cladosphaeria, Cladosporium, Cladosterignia, Claobtrichum, Clarkeinda, Clasterosporium, Clathrella, Clathridium, Clathrococcum, Clathrogaster, Clathroporina, Clathrospora, Clathrotrichum, Clathrus, Claudopus, Claussenomyces Claustula, Clavaria, Clavariaceae, Clavariopsis, Clavariopsis, Claviceps, Clavogaster, Clavularia, Clavulinopsis, Cleistophoma, Cleistosoma, Cleistosphaera, Cleistotheca, Cleistothecopsis, Clematomyces, Cleptomyces, Clidiomyces, Cliniconidium, Clinterium, Clintoniella, Cliostomum, Clistophoma, Clistosoma, Clistosphaera, Clistotheca, Clistothecopsis, Clithris, Clitocybe, Clitopilus, Clonostachyopsis, Clonostachys, Closteraleurosporia, Closterosporia, Clypeochorella, Clypeodiplodina, Clypeolella, Clypeolina, Clypeolina, Clypeolum, Clypeoporthc, Clypeoporthela, Clypeopycnis, Cypcoseptoria, Clypeosphaeria, Clypeostignia, Clypeostroma, Clypeothecium, Clypeotrabutia, Coccidiascus, Coccidiodes, Coccidomyces, Coccidophthora, Cocciscia, Coccobotrys, Coccocarpia, Coccochora, Coccochorela, Coccodiella, Coccodinium, Coccodiscus, Coccodothella, Coccodothis, Coccoidea, Coccoidella, Coccomycella, Coccomyces, Coccomycetela, Cocconia, Cocconiopsis, Coccopeziza, Coccophacidium, Coccospora, Coccosporella, Coccosporium, Coccostroma, Coccostromopsis, Coccotrema, Coelographium, Coelomyces, Coelomycidium, Coelosphaeria, Coemansia, Coemansiela, Coenogonium, Coleodictyospora, Coleodictys, Coleonaema, Coleophoma, Coleopuccinia, Coleosporium, Coleroa, Collacystis, Collema, Collemaceae, Collemis, CoUemodes, Collemopsidium, Colletomanginia, Colletotrichella, Colletotrichopsis, Colletotrichum Collodochium, Collonaema, Collonaemella, Collybia, Collyria, Colpoma, Coipomella, Columnophora, Columnothyrium, Colus, Combea, Comesia, Comoclathris, Complectoria, Compsomyces, Confervales, Conida, Conidiascus, Conidiobolus, Coniella, Coniocarpum, Coniochaeta, Coniocybe, Coniodictyum, Coniophora, Coniophorella, Conioscypha, Coniosporium, Coniothecium, Coniothyrella, Coniothjriella, Coniothyrina, Coniothyrimila, Coniothyriopsis, Coniothyriopsis, Coniothyris, Coniothyrium, Conoplea, Conostroma, Conotheciella, Conotrema, Constantinella, Cookeina, Cookella, Copelandia, Copranophilus, Coprinopsis, Coprinus, Coprolepa, Cora, Corallodendrum, Corallomyces, Coraliomycetella, Cordana, Cordelia, Cordierites, Corditubera, Cordyceps, Corella, Coremiella, Coremium, Coreomyces, Corethromyces, Corethropsis, Cornicularia, Comiculariella, Cornucopiella, Cornuella, Comularia, Corollium, Corollospora, Coronella, Coronophora, Coronophorella, Coronotelium, Corticium, Cortinarius, Corymbomyces, Coryne, Corynelia, Coryneliaceae, Coryneliella, Corynespora, Corynetes, Coryneum, Coscinaria, Cosdnopeltis, Cosmariospora, Coutinia, Couturea, Crandallia, Craterellus, Craterocolla, Creomelanops, Creonectria, Creosphaeria, Creothyrium, Crepidotus, Criella, Crinula, Crinula, Criserosphaeria, Cristulariella, Crocicreas, Crocynia, Cronartium, Crossopsora, Crotone, Crotonocarpia, Crucibulum, Crumenula, Cryphonectna, Cryptascus, Cryptica, Cryptobasidium, Cryptoceuthospora, Cryptocline, Cryptococcus, Cryptocoryneum, Cryptoderis, Cryptodiaporthe, Cryptodidnymosphaeria, Cryptodiscus, Cryptoleptosphaeria, Cryptomela, Cryptomycella, Cryptomyces, Cryptomycina, Cryptonectriopsis, Cryptopeltis, Cryptopeltosphaeria, Cryptopezia, Cryptophaella, Cryptophallus, Cryptoporus, Cryptopus, Cryptorhynchella, Cryptorhynchella, Cryptosphaerella, Cryptosphaeria, Cryptosphaerina, Cryptospora, Cryptosporella, Cryptosporina, Cryptosporiopsis, Cryptosporium, Cryptostictella, Cryptostictis, Cryptothecium, Cryptothele, Cryptothelium, Cryptovalsa, Ctenoderma, Ctenomyces, Cubonia, Cucurbidotliis, Cucurbitaria, Cucurbitariella, Cudonia, Cudoniella, Cutininghaniella, Cunninghamia, Curreya, Curreyella, Cuticularia, Cutomyces, Cyanobaeis, Cyanocephalum, Cyanochyta, Cyanoderma, Cyanophomella, Cyanospora, Cyathicula, Cyathus, Cycloconium, Cycloderma, Cydodomus, Cyclodothis, Cyclographa, Cyclomyces, Cycloschizella, Cycoschizum, Cycdostoniella, Cycdotheca, Cycdothyrium, Cylindrina, Cylindrium, Cylindrocarpum, Cylindrocephalum, Cylindrocdadium, Cylindrocolla, Cylindrodendrum, Cylindrophora, Cylindrosporelia, Cylindrosporium, Cylindrothyrium, Cylindrotrichum, Cylomyces, Cyniatella, Cyphelium, Cyphella, Cyphellomyces, Cyphellopycnis, Cyphina, Cyphospilea, Cystingophora, Cystodendrum, Cystolobis, Cystomyces, Cystophora, Cystopsora, Cystopus, Cystospora, Cystotelium, Cystotheca, Cystothyrium, Cystotricha, Cytidia, Cytodiplospora, Cytogloeum, Cytonaema, Cytophoma, Cytoplacosphaeria, Cytoplea, Cytosphaera, Cytospora, Cytosporella, Cytosporina, Cytosporium, Cytostaganis, Cytostaganospora, Cytotriplospora, Cyttaria, Cyttariaceae, Dacrymycella, Dacryobolus, Dacryodochium, Dacryomitra, Dacryomyces, Dacryomycetaceae, Dacryopsella, Dacryopsis, Dactylaria, Dactylella, Dactylina, Dactylium, Dactylomyces, Dactylosporium, Daedalea, Daldinia, Daleomyces, Dangeardia, Dangeardiella, Darbishirella, Darluca, Darlucis, Darwiniella, Dasybolus, Dasypezis, Dasyphthora, Dasypyrena, Dasyscypha, Dasyscyphae, Dasyscyphella, Dasysphaeria, Dasyspora, Dasysticta, Dasystictella, Davincia, Davinciella, Davisiella, Dearnessia, Debaryella, Debaryonyces, Deconica, Delacourea, Delastria, Delastriopsis, Delitschia, Delitschiella, Delortia, Delphinella, Delpinoella, Delpontia, Dematiaceae, Dematium, Dendrocladium, Dendrocyphella, Dendrodochium, Dendrodomus, Dendroecia, Dendrogaster, Dendrographa, Dendrographium, Dendrophoma, Dendrosphaera, Dendrostilbella, Dendrothele, Dendryphiella, Dendryphium, Dermatea, Dermateaceae, Dermatella, Dermatina, Dermatiscum, Dermatocarpae, Dermatocarpum, Dermatodothis, Dermophyta, Desmazierella, Desmella, Desmidiospora, Desmopatella, Desmotascus, Detonia, Deuteromycetes, Dexteria, Diabole, Diachora, Diachorella, Dialhypocrea, Dialonectria, Diaphanium, Diaporthe, Diaporthella, Diaporthopsis, Diarthonis, Diathryptum, Diatractium, Diatrype, Diatrypella, Dibaeis, Dibelonis, Diblastospermella, Diblepharis, Dicaeoma, Dicarpella, Dichaena, Dichaenopsis, Dichaetis, Dichirinia, Dichlaena, Dichlamys, Dichomera, Dichomyces, Dichoporis, Dichosporium, Dichostereum, Dichothrkz Dichotomella, Dichotonium, Dicoccum, Dicollema, Dicranidium, Dicranophora, Dictyobole, Dictyocephalus, Dictyochaeta, Dictyochora, Dictyochorella, Dictyodothis, Dictyographa, Dictyolus, DictyomoUis, Dictyonella, Dictyonema, Dictyonia, Dictyopeltineae, Dictyopeltis, Dictyophora, Dictyorinis, Dictyosporium, Dictyothyriella, Dictyothyrina, Dictyothyrium, Dictyuchus, Dicyma, Didothis, Didymaria, Ddymariopsis, Ddymascella, Didymascella, Didymascina, Didymascus, Didymella, Didymellina, Didymellopsis, Didymobotryopsis, Didymobotrys, Didymobotryum, Dymochaete, Didymochlamys, Didymochora, Didymocladium, Ddymocoryne, Ddymopsamma, Dymopsis, Didymopsora, Didymosphaeria, DAvmosporiella, Didymosporina, Didymosporis, Didymosporium, Didymostilbe, Didymothozetia, Dwymotricha, Ddymotrichum, Diedickea, Diedickella, Dielsiella, Dietelia, Digraphis, Dilophia, Dilophospora, Dimargaris, Dimeriella, Dimerellopsis, Dimerina, Dimerinopsis, Dimeriopsis, Dimerisma, Dimerium, Dimeromyces, Dimerosporiella, Dimerosporina, Dimerosporiopsis, Dimerosporium, Dimorphomyces, Dinemasporiella, Dinemasporiopsis, Dinemasporis, Dinemasporium, Dioecomyces, Dioranotropis, Diorchidium, Diphaeis, Diphaeostica, Diphanis, Diphanosticta, Diphloeis, Diplocarpa, Diplocarpum, Diploceras, Diplochora, Diplochorella, Diplocladium, Diplococcium, Diplocryptis, Diplocystis, Diplodascus, Diploderma, Diplodia, Diplodiella, Diplodina, Diplodinis, Diplodiopsis, Diplodothiorella, Diplogramma, Diploidium, Diplomyces, Diplonaevia, Diploospora, Diplopeltis, Diplopetis, Diplopeltopsis, Diplophyctis, Diplophysa, Diploplacis, Diploplacosphaeria, Diploplenodomopsis, Diploplenodomus, Diplorhinotrichum, Diploschistes, Diplosderophoma, Diplosphaerella, Diplosporis, Diplosporium, Diplostephanus, Diplotheca, Diplotomma, Diplozythia, Diplozythiella, Diporina, Dipyrenis, Dirina, Dirinae, Dirinaria, Dirinastrum, Disaeta, Discella, Dscellaceae, Discellae, Discina, Disciseda, Discocera, Discochora, Discocolla, Discocyphella, Discodiaporthe, Discodothis, Discofusarium, Discogloeum, Discomycella, Discomycopsella, Discomycopsis, Discosia, Discosiella, Discosphaerina, Discosporella, Discosporiella, Discosporiopsis, Discosporium, Discostroma, Discostromella, Discotheciella, Discothecium, Discozythia, Discula, Disculina, Disperma, Dispira, Dissophora, Distichomyces, Dithelopsis, Dithozetia, Ditiola, Ditopella, Ditremis, Ditylis, Doassansia, Doassansiopsis, Doratomyces, Dothichiza, Dothichloe, Dothiclypeolum, Dothidastens, Dothidasteroma, Dothidasteromella, Dothidea, Dothideaceae, Dothideae, Dothideales, Dothidella, Dothideodiplodia, Dothideopsella, Dothideovalsa, Dothidina, Dothidotthia, Dothiopsis, Dothiora, Dothiorae, Dothiorellina, Dothiorina, Dothisphaeropsis, Dothithyrella, Dothophaeis, Drepanoconis, Drepanopeziza, Drepanospora, Dubiomyces, Ductifera, Dufourea, Duplicaria, Duportella, Durandia, Durandiomyces, Durella, Dussiella, Dyslachnum, Dyslecanis, Dysrhynchis, Dysticta, Dystictina, Earlea, Ecchyna, Eccilia, Echidnodella, Echidnodes, Echinobotryum, Echinodontium, Echinodothis, Echinophallus, Echinothedum, Echusias, Ectinomyces, Ectosphaeria, Ectosticta, Ectostroma, Ectotrichophytum, Ectrogella, Eichleriella, Eidamella, Elachopeltis, Elaeodema, Elaphomyces, Elaphomycetaceae, Elasmomyces, Elateromyces, Eleutheris, Eleutheromycella, Eleutheromyces, Eleutherosphaera, Elisiella, Ellisiodothis, Elmeria, Elmerina, Elmerococcum, Elsinoae, Elsinoe, Emericella, Empusa, Empusaceae, Enantiothamnus, Enarthromyces, Encephalcographa, Enchnoa. Enchnosphaeria, Encoelia, Encoeliella, Endobasidium, Endoblastoderma, Endobotrya, Endobotryella, Endocalyx Endocarpum, Endocena, Endocladis, Endococcus, Endoconidiophora, Endoconidium, Endocoryneum, Endocycia, Endodermophytum, Endodesmia, Endodothella, Endodothiora, Endogloea, Endogonaceae, Endogone, Endogonella, Endomyces, Endomycetaceae, Endophragmia, Endophyllachora, Endophylloides, Endophyllum, Endoscypha, Endospora, Endostigme, Endothia, Endothiella, EndoxyIa, Endoxylina, Endyllium, Englerodothis, Engleromyces, Englerula, Englerulaceae, Englerulaster, Enterodictyum, Enterostigma, Enthallopycnidium, Entodesmium, Entoleuca, Entoloma, Entomopatella, Entomophthora, Entomosporium, Entonaema, Entopeltis, Entophyctvs, Entorhiza, Entosordaria, Entyloma, Eocronartium, Eolichen, Eomycenella, Eosphaeria, Eoterfezia, Ephebae, Ephebe, Ephebeia, Ephelidium, Ephelina, Epheliopsis, Epheliopsis, Ephelis, Epibotrys, Epichloe, Epiclinium, Epicoccum, Epicorticium, Epicymatia, Epicyta, Epidermidophyton, Epidermophytum, Epidochiopsis, Epidochium, Epigloea, Epilichen, Epinectria, Epipeltis, Epiphora, Epiphyma, Epipolaeum, Episoma, Episphaerella, Epistigme, Epithele, Epochnium, Eremascus, Eremotheca, Eremothecella, Eremothecium, Erikssonia, Erinella, Erioderma, Eriomene, Eriomenella, Eriomycopsis, Eriopeziza, Eriosphaeria, Eriospora, Eriosporangium, Eriosporella, Eriosporina, Eriothyrium, Erostella, Erostrotheca, Erysiphaceae, Erysiphe, Erysiphella, Eryiphopsis, Ersiphopsis, Erythrocarpum, Euacanthe, Euantennaria, Eubelonis, Eucantharomyces, Euchaetomella, Eucorethromyces, Eucyphelis, Eudarluca, Eudimeriolum, Euhaplomyces, Eumela, EumoUisiae, Eumonoecomyces, Eupelte, Eupropolella, Eupropolis, Eurotiaceae, Eurotiella, Eurotiopsis, Eurotium, Euryachora, Eurychasma, Eurytheca, Eustictidae, Euthryptum, Eutorula, Eutorulopsis, Eutypa, Eutypella, Eutypopsis, Euzodiomyces, Everhartia, Evemia, Epemiopsis, Exarmidium, Exascaceae, Exascus, Excioconis, Excipula, Excipulaceae, Excipularia, Excipulella, Excipulina, Exidia, Exidiopsis, Exilospora, Exobasidiopsis, Exobasidium, Exogone, Exophoma, Exosporella, Exosporina, Exosporina, Exosporium, Exotrichum, Fabraea, Fairmania, Fairmaniella, Falcispora, Farlowiella, Farriola, Farysia, Favillea, Favolus, Fems jonia, Fenestella, Feracia, Ferrarisia, Filoboletus, Fimetaria, Fioriella, Fischerula, Fistulina, Fistulinella, Flageoletia, Flaminia, Flammula, Fleischeria, Fleischhakia, Floccomutinus, Fomes, Fominia, Forssellia, Fouragea, Fracchiaea, Fragosoa, Fragosoella, Fragosphaeria, Friesula, Frommea, Fuckelia, Fuckelina, Fulininaria, Fumago, Fumagopsis, Fumagospora, Fusariella, Fusarium, Fusella, Fusicladiella, Fusicladium, Fusicoccum, Fusicolla, Fusidium, Fusisporella, I Fusoma, Gaillardiella, Galactinia, Galera, Gallowaya, Galzrma, Gambleola, Gamonaemella, Gamospora, Gamosporella, Ganoderma, Gastroboletus, Gautieria, Geaster, Geasteroides, Geasteropsis, Geisleria, Gelatinosporis, Gelatinosporium, Geminispora, Genabea, Genea, Geoglossae, Geoglossum, Geolegnia, Geopora, Geopyxis, Geotrichum, Gerwasia, Gibbera, Gibberella, Gibberidea, Gibellia, Gibellina, Gibellula, Gibsonia, Gilletia, Gilletiella, Gillotia, Giulia, Glaziella, Glenospora, Gliobotrys, Gliocephahs, Gliocladium, Gliocadochium, Gliomastix, Glischroderma, GbObaria, Globulina, Gloeocalyx, Gheocephala, Gloeocystidium, Gloeodes, Gloeoglossum, Gloeopeniophora, Gloeopeziza, Gloeoporus, Gloeosoma, Gloeosphaera, Gloeosporidiella, Gloeosporidina, Gloeosporidium, Gloeosporiella, Gloeosporina, Gloeosporiopsis, Gloeosporium, Gloeothele, Glomerella, Glomerula, Glomerularia, Glomus, Gloniella, Gloniopsis, Glonium, Glossodium, Glutinium, Gycophila, Glyphis, Glypholecia, Gnomonia, Gnomoniella, Gnomonina, Gnomoniopsis, Godfrinia, Godronia, Godroniella, Godroniopsis, Gomphidius, Gomphillus, Gonapodya, Gonatobotrys, Gonatobotrytae, Gonatobotryum, Gonatorhodis, Gonatorhodum, Gongromeriza, Gongylia, Gonisporium, Gonisporiuni Gonohymenia, Gonolecania, Gonothecis, Gonothecium, Gonyella, Gonytrichum, Goplana, Gorgoniceps, Grallomyces. Grammothele, Grandinia, Grandiniella, Granularia, Graphidaceae, Graphidae, Graphidium, Graphina, Graphinella, Graphiola, Graphiolaceae, Graphiopsis, Graphiothecium, Graphis, Graphium, Graphyllium, Griggsia, Griphosphaerella, Griphosphaeria, Griphosphaerioma, Groveola, Grubyella, Gueguenia, Guelichia, Guepinia, Guignardia, Guignardiella, Guillermondia, Giiillermondia, Guttularia, Guttularia, Gyalecta, Gyaectae, Gymnascaceae, Gymnascales, Gymnascus, Gymnoconia, Gymnoderma, Gymnodochium, Gymnoglossum, GymnograpHa_Gyninomyces, Gymnopeltis, Gymnosporangium, Gymnotelium, Gyrocephalus, Gyroceras, GyrocoUema, Gyrocratera, Gyrodon, Gyromitra, Gyrophora, Gyrophorae, Gyrophragmium, Gyrostomum, Gyrostroma, Habrostictis, Hadotia, Hadronema, Hadrotrichum, Haematomma, Haematomyces, Haematomyxa, Hainesia, Halbania, Halbaniella, Halbanina, Halobyssus, Halonia, Halstedia, Hamaspora, Hamasporella, Hansenia, Hanseniospora, Hansenula, Hapalocystis, Hapalophragmium, Hapalosphaeria, Haplaria, Haplariella, Haplariopsis, Haplariopsis, Haplobasidium, Haplodothella, Haplodothis, Haplographium, Haplolepis, Haplomela, Haplomyces, Haplopeitineae, Haplopeltis, Haplophyse, Haplopyrenula, Haplopyxis, Haploravenelia, Haplosporangium, Haplosporella, Haplosporidium, Haplosporium, Haplostroma, Haplotheciella, Haplothecium, Haplothelium, Haplotrichum, Haplovalsaria, Haraea, Hariotia, Hariotula, Harknessia, Harknessiella, Harpagomyces, Harpidium, Harpocephalum, Harpochytrium, Harpographium, Harposporella, Hartiella, Hartigiella, Harziella, Hassea, Hebeloma, Helicia, Helicobasidium, Helicobasis, Helicocephalum, Helicodendrum, Helicodesmus, Helicogloea, Helicoma, Helicomyces, Helicopsis, Helicosporangium, Helicosporium, Helicostilbe, Helicostylum, Helicotrichum, Helicoum, Heliomyces, Heliscus, Helminthocarpum, Helminthophana, Helminthosphaeria, Helminthosporium, Helolachnum, Helostroma, Helotiaceae, Helotiae, Helotiopsis, Helotium, Helvella, Helvellaceae, Helvellae, Hemidothis, Hemigaster, Hemiglossum, Hemileia, Hemileiopsis, Hemisphaeriaceae, Hemispora, Hendersonia, Hendersoniella, Hendersonina, Hendersoninula, Hendersoniopsis, Hendersonula, Henningsia, Henningsiella, Henningsina, Henningsomyces, Henriquesia, Heppia, Heppiae, Heptameria, Heptasporium, Hercospora, Hericium, Hermatomyces, Herpobasidium, Herpocladiella, Herpocladium, Herpomyces, HerpothriK Herpotrichia, Herpotrichiella, Herpotrichiopsis, Heterobasidium, Heterobotrys, Heterobotrys, Heterocarpum, Heterocephalum, Heteroceras, Heterochaete, Heterochaetella, Heterochlamys, Heterodea, Heterodothis, Heteromyces, Heteronectria, Heteropatella, Heteropera, Heterophracta, Heteroplegma, Heterosphaeria, Heterosporium, Hetcrotcxtus, Hexagonella, Hexagonia, Heydenia, Heydeniopsis, Hiatula, Himantia, Hippoperdum, Himeola, Himeolina, Hirsutella, Hirundinaria, Histoplasma, Hobsonia, Hoehneliella, Hoehnelogaster, Hoehnelomyces, Hodcomyces, Hodocoenis, Holocyphis, Hodothelis, Holstiella, Holwaya, Hodwayella, Homopsella, Homostegia, Hormiactella, Hormiactina, Hormiactis, Honiiisciopsis, Hormiscium, Horniococcus, Hormodendrum, Hormomyces, Hormonema, Hormopeltis, Hormosperma, Hormothecium, Hormylium, Hueella, Humaria, Humariella, Humarina, Husseya, Hyalasterina, Hyalinia, Hyaloceras, Hyalocrea, Hyalocurreya, Hyalodema, Hyaloderma, Hyalodermella, Hyalodictyum, Hyalodothis, Hyalomeliolina, Hyalopeziza, Hyalopsora, Hyalopus, Hyaloria, Hyaloscypha, Hyalosphaera, Hyalotexis, Hyalotheles, Hyalothyris, Hydnaceae, Hydnangium, Hydnobolites, Zll Hydnochaete, Hydnochaete, Hydnocystis, Hydnodon, Hydnofomes, Hydnotrya, Hydnotryopsis, m Hydnum, Hydraeomyces, Hydrogera, Hydroncctnia, Hydrophilomyces, Hydrophora, Hydrothyria, Hygrophorus, Hymenella, Hymenobactrum, Hynienoboliis, Hymenochaete, Hymenogaster, Ii Hymenogastraceae, Hymenogramme, Hymenopsis, Hymenoscypha, Hymenula, Hyperomyxa, Hyperphyscia, Hyperus, Hypha, Hyphaster, Hyphochytriinii, Hyphoderma, Hyphodiscus, Hypholoma, Hyphoscypha, Hyphosoma, Hyphostereum, Hypocapnodium, Hypocelis, Hypocenia, Hypochnaceae, Hypochnus, Hypocopra, Hypocrea, Hypocreaceae, Hypocrella, Hypocreodendrum, Hypocreophis, Hypocreopsis, Hypoderma, Hypodermella, Hypodermellna, Hypodermina, Hypodermina, Hypodermium, Hypodermopsis, Hypogloeum, Hypolyssus, Hypomyces, Hypomycopsis, Hyponectria, Hypoplegma, Hypoplegma, Hypospila, Hypospilina, Hypostegium, Hypostigine, Hypoxylina, Hypoxylopsis, Hypoxylum, Hysterangium, Hysteiaceae, Hysteridiuil, Hysterium, Hysteroglonium, Hysterographium, Hysteromyxa, Hystcropatella, Hysteropeltella, Hysteropeziza, Hysteropezizela, Hysteropsis, Hysteropsis, Hysterostegiella, Hysterostoma, Hysterostomella, Hysterostomina, Icmadophila, Idiomyces, juhya, Ileodictyum, Illosporium, Indiella, Ingaderia, Inocybe, Inocyclus, Inzengaea, lotidea, Irene, Irenina, Irenopsis, Iridionia, Irpex, Isaria, Isariella, Isariopsis, Ischnostroma, Isipinga, Isoachlya, Isomunkia, Isomyces, Isothea, Isthmospora, Itajahya, Ithyphallus, laapia, lackya, laczewskia, laczewskiella, laffuela, lahniella, lainesia, lanospora, lanseela, lansia, laponia, laraia, lattaea, lenmania, lohansonia, lola, lonaspis, Julella, KKabatia, Kabatiella, Kalchbrennera, Kalmusia, Karschia, Karstenia, Karstenula, Kawakamia, Keissleria, Keisslerella, Keisslerina, Keithia, Kellermannia, Kerminicola, Khekia, Kickxella, Kirschsteinia, Kirschsteiniella, Kiastospora, Klebahnia, Keidiomyces, Kmetia, Kneiffia, Koerberia, Konenia, Konradia, Koordersiella, Kordyana, Kordyanella, Kretschmaria, Kriegeria, Kriegerella, Kuehneola, KuUhemia, Kunkelia, Kuntzeomyces, Kupsura, Kusanoa, Kusanobotrys, Kusanoopsis, Laaseonipces, Laboulbenia, Laboulbeniaceae, Laboulbeniales, Labrella, Labridium, Lacellina, Lachnaster, Lachnea, Lachnella, Lachnellula, Lachnocaulum, Lachnocladium, Lachnodochium, Lachnum, Lactaria, Lactariopsis, Lactarius, Laestadia, Laestadiella, Lagena, Lagenidiopsis, Lagenidium, Lageniformia, Lagerheimia, Lagynodella, Lahmia, Lambertella, Lambottiela, Lambro, Lamia, Lamprospora, Lamyella, Langloisula, Lanomyces, Lanopila, Lanzia, Laquearia, Laschia, Lasiella, Lasiobelonis, Lasiobelonium, Lasiobolus, Lasiobotrys, Lasiodiplodia, Lasionectria, Lasiophoma, Lasiosordaria, Lasiosphaera, Lasiosphaeria, Lasiosphaeris, Lasiostemma, Lasiostictis, Lasiostroma, Lasiothyrium, Lasmenia, Lasmeniella, Latrostium, Latzelia, Laurera, Lauterbachiella, Leandria, Lecanactidae, Lecanactis, Lecania, Lecaniascus, Lecanidion, Lecaniopsis, Lecanora, Lecanorae, Lecanosticta, Lecidea, Lecideaceae, Lecideae, Lecideopsella, Lecideopsis, Lecidopyrenopsis, Lecioglyphis, Leciographa, Leciophysma, Lecithium, Lecopyrenopsis, Leeina, Leiosepium, Leiosphaerella, Lelujn, Lemalis, Lembosia, Lembosiella, Lembosina, Lembosiodothis, Lembosiopsis, Lemmopsis, Lemonniera, Lempholemma, Lentinus, Lentodiopsis, Lentodium, Lentomita, Lentomitella, Lenzites, Leotia, Leotella, Lepidella, Lepidocollema, Lepidogium, Lepidoeptogium, Lepiota, Lepolichen, Lepraria, Leprieurina, LeprocoUema, Leptascospora, Lepteutypa, Leptinia, Leptobelonium, Leptochlamys Leptocoryneum, Leptocrca, Leptodermella, Leptodothiora, Leptodothis, Leptogidium, Leptogiopsis, Leptogium, Leptoglossum, Leptographium, Leptolegnia, Leptomassaria, Leptomelanconium, Leptomeliola, Leptomitae, Leptomitus, Leptonia, Leptopeltella, Leptopeltina, Leptopeltis, Leptopeziza, Leptophacidium, Leptophoma, Leptophyma, Leptopuccinia, Leptorhaphis, Leptosacca, Leptosillia, Leptosphaerella, Leptosphaeria, Leptosphaeropsis, Leptosphaerulina, Leptospora, Leptosporella, Leptosporium, Leptosporopsis, Leptostroma, Leptostromaceae, Leptostromella, Leptothyrella, Leptothyrina, Leptothyrium, Leptotrema, Leptotrichum, Leptoxyphium, Letendraea, Letharia, Lethariopsis, Leucangium, Lcucobolites, Leucoconis, Leucoconius, Leucocrea, Leucocytospora, Leucodochium, Leucogaster, Leucopaxillus, Leucopezis, Leucophleps, Leucophomopsis, Leucostoma, Leucothyridium, Leveillella, Leveihna, Leveillinopsis, Leveillula, Levieuxia, L ibertella, L ibertiella, L ibertina, Lichenoconium, Lichenopeltella, Lichenophoma, Lichenosticta, Lichenylium, Lichina, Lichinae, Lichinella, Lichinodium, Lichtheimia, Licopolia, Ligniella, Ligniera, Ldliputia, Lnimacinia, Lnimadnia, Limaciniella, Lnimaciniopsis, Limnaeomyces, Lindauella, Lindauomyccs, Lindauopsis, T, indrothia, Linearistroma, Linhartia, Linkiclla, Linocarpum, Linochora, Linochorella, Linodochium, Linospora, Linostoma, Linostomella, Linostroma, Linotexis, Lipospora, Lisea, Lisiella, Listeromyces, Lithoecea, Lithographa, Lithothelium, Litschaueria, Lituaria, Lizonia, Lizoniella, Lloydiella, Lobaria, Lobarina, Locellina, Loculistroma, Lojkania, Lonchospermella, Longia, ZZI Longoa, Lopadiopsis, Lopadium, Lopadostoma, Lopharia, Lophidiopsis, Loplidium, Lophiella, Lophionema, Lophiosphaera, Lophiostoma, Lophiostomaceae, Lophiotrema, Lophiotricha, Lophium, Lophodermela, Lophodermium, Lophodermopsis, Lophophytum, Loramyces, Loranthomyces, Ludwigiella, Lulworthia, Lycogalopsis, Lycoperdaceae, Lycoperdales, Lycoperdellon, Lycoperdopsis, Lycoperdum, Lyvnella, Lysospora, Lysurus, MMacalpinia, Macbridella, Macowaniella, Macowanites, Macrobasis, Macrochytrium, Macroderma, Macrodiaporthe, Macrodiplis, Macrodiplodia, Macrodiplodiopsis, Macrophoma, Macrophomela, Macrophomina, Macrophomopsis, Macroplodella, Macropodia, Macroseptoria, Macrospora, Macrosporium, Macrostilbum, Madurella, Magnusia, Magnusiella, Magnusiomyces, Maireella, Malacodermis, Malacosphaeria, Malassezia, Malbranchea, Malmeomyces, Mamiana, Mamianella, Manginia, Manginula, Manilaea, Mapea, Marasniiopsis, Marasmius, Maravalia, Marchalia, Marchaiella, Marcosia, Maronea, Marsonia, Marsoniella, Marsonina, Martellia, Martensella, Martindalia, Martinella, Massalongia, Massalongiella, Massalongina, Massaria, Massariella, Massariellops, Massarina, Massarinula, Massariopsis, Massariovalsa, Masseea, Masseella, Massospora, Mastigocladium, Mastigonema, Mastigonetrum, Mastigosporella, Mastigosporium, Mastodia, Mastomyces, Matruchotia, Mattirolia, Matula, Maublancia, Mauginiella, Maurodothella, Maurodothis, Maurya, Maxillospora, Mazzantia, Alazzantiella, Medeolaria, Medusomyces, Medusulina, Megalonectria, Megalopsora, Megaloseptoria, Megalospora, Melachroia, Melampsora, Melampsoraceae, Melampsorella, Melampsoridium, Melampsoropsis, Melampydium, Melanconiaceae, Melanconiales, Melanconiella, Melanconiopsis, Melanconis, Melanconium, Melanidium, Melanobasidium, Melanobasis, Melanobotrys, Melanochlamys, Melanodiscus, Melanogaster, Melanographium, Melanomma, Melanomyces, Melanoplaca, Melanops, Melanopsamma, Melanopsammella, Melanopsammina, Melanopsammopsis, Melanopsichium, Melanosphaeria, Melanospora, Alelanosporopsis, Melanostroma, Melanotaenium, Melanotheca, Melasmia, Melaspilea, Melastiza, Melchiora, Meliola, Meliolaster, Meliolidium, Meliolina, Meliolinopsis, Melioliphila, Meliolopsis, Melittosporiella, Melittosporiopsis, Melittosporis, Melittosporium, Melogramma, Melomastia, Melophia, Memnoniella, Mendogia, Menezesia, Menispora, Menoidea, Merarthonis, Meria, Meringosphaeria, Merismatium, Merismella, Merodontis, Merophora, Meroplacis, Merorinis, Merostictina, Merostictis, Merrilliopeltis, Merulius, Mesniera, Mesobotrys, Mesonella, Mesophellia, Mesopsora, Metabotryum, Metacapnodium, Metachora, Metacoleroa, Metadothella, Metameris, Metanectria, Metasphaeria, Metathyriella, Methysterostomella, Metraria, Michenera, Micranthomyces, Micrascus, Microbasidium, Microcallis, Microcera, Microclava, Microcyclella, Microcyclus, Microdiplodia, Microdiscula, Microdiscus, Microdochium, Microdothella, Microglaena, Microgloeum, Microglossum, Micrographa, Micromastia, Micromyces, Micromycopsis, Micromyriangium, Micronectria, Micronectriella, Micronectriopsis, Micronegeria, Micropeltaceae, Micropeltella, Micropeltis, Micropeltopsis, Micropera, Microperella, Microphiale, Microphiodothis, Micropodia, Micropsalliota, Micropuccinia, Micropyrenula, Microscypha, Microspatha, Microsphaera, Microsphaeropsis, Microsporella, Microsporum, Microstelium, Microsticta, Microstroma, Microthecium, Microthelia, Microtheliopsis, Microthyriaceae, Microthyriales, Microthyrieae, Microthyrella, Microthyriolum, Microthyris, Microthyrites, Microthyrium, Microtyle, Microtypha, Microxyphium, Microxyphiella, Micula, Midotiopsis, Midotis, Milesia, Milesina, Milowia. Mindemella, Minksia, Mitochytridium, Mitochytrium, Mitopeitis, Mitosporium, Mitromyces, Mitrula, Mitruliopsis, Miyabella, Miyagia, Mryakeanipces, Miyoshia, Mroshiella, Moelleriella, Moelleroclavus, Moellerodiscus, Moeszia, Moesziella, Mohortia, Molleriella, Molliardia, Mollisia, MoUisiaceae, Molisiella, MoUisiopsis. Monacrosporium, Monascaceae, Monascostroma, Monascus, Monilia, Moniliaceae, Moniliales, Moniliopsis, Monilochaetes, Monoblastia, Monoblepharidaceae, Monoblephariopsis, Monoblepharis, Monochaetia, Monoecomyces, Monogrammia, Monographella, Monographus, Monopodium, Monopus, Monopycnis, Monorhiza, Monorhizina, Monospora, Monosporella, Monosporidium, Monosporiella, Monosporium, Monostichella, Monotospora, Monotrichum, Montagnellina, Montagnina, Montagnites, Montagnula, Montemartinia, Montoyella, Morchella, Morenella, Morenina, Morinia, Moriola, Moriolae, Mortierella, Mortierellae, Moschomyces, Moutoniella, Muchmoria, Muciporus, Mucor, Mucoraceae, Mucorae, Mucronella, Mucronoporus, Mucrosporium, Muellerella, Muiaria, Muiogone, Multipatina, Munkia, Munkiella, Munkiodothis, Murashkinskka, Mutinus, Mycaureola, Myceliophthora, Myceloderma, Mycelophagus, Mycena, Mycenastrum, Mycobacidia, Mycobacillaria, Mycobilimbia, Mycoblastus, Mycocalicium, Mycocitrus, Mycocladus, Mycodendrum, Myroderma, Myrogala, Mycogone, Mycolangloisia, Mycoecidea, Mycoecis, Mycomalus, Mycophaga, Mycopharus, Mycoporaceae, Mycoporellum, Mycoporns, Mycoporum, Myropyrcmila, Mycorhynchella, Mycorhynchus, Mycosphaerella, MycosphaercUopsis, Mycosticta, Mycosyrinx, j\lycotorula, Mycovellosiella, Myelosperma, Myiocoprella, Myiocoprum, Mylittopsis, Myriadoporus, Myriangella, Myriangiaceae, Myriangiae, Myriangina, Myrianginella, Myriangiopsis, Myriangium, Myridium, Myrelina, Myrillium, Myrioblepharis, Myriococcum, Myrioconium, Myrioconiuni Myriogenis, Myriogenospora, Myriolecis, Myriophysa, Myriophysella, Myriopyxis, Alyriostigina, Myrmaedella, Myrmaecium, Myrmecocystis, Myrotheciella, Myrothecium, Mystrosporium, Mytilidium, Myxasterina, Myxocycdus, Myxodictyum, Myxodiscus, Myxofusicoccum, Myxolibertella, Alyxomycidium, Myxomyriangis, Myxomyriangium, Myxonema, Myxophacidiella, Myxophacidiuni Myxormia, Myxosporella, Myxosporina, Myxosporium, Myxotheca, Myxothecium, Myxothyrium, Myxotrichella, Myxotrichum, Myzocytium, Nadsonia, Naegelia, Naeg- eliella, Naemacyclus, Naematelia, Naemosphaera, Nacmosphaerella, Naemospora, Naetrocymbe, Naevia, Naeviella, Napicladium, Napomyces, Naucoria, Naumovia, Necator, Necium, Nectaromyccs, Nectria, Nectriella, Nectriella, Nectrioidaceae, Nectriopsis, Negeriella Nemastroma, Nematogonium, Nematospora, Nematosporangium, Nematostigma, Neinatostoma, Nematothedum, Nemozythiella, Neoarcangelia, Neobarclaya, Neobulgaria, Neocosmospora, Neofabraea, Neohendersonia, Neohenningsia, Neoheppia, Neohoehnelia, Neokeissleria, Neolamya, Neolecta, Neoniichcdia, Neoncctria, Neopatella, Neopeckia, Neophoma, Neopdacosphaeria, Neoravenelia, Neorehmia, Neosaccardia, Neoskofitzia, Neosphaeropsis, Neostomella, Neotrichophytum, Neotrotteria, Neottiella, Neottiopezis, Neottiospora, Neottiosporella, Neottiospons, Neovcnturia, Neovossia, Neozimmermannia, Nephlyctis, Nephroma, Nephromium, Nephromopsis, Nephrospora, Ncpotatiis, Nesolechia, Nidula, Nidularia, Nidulariaceae, Nielsenia, Nesslella, Nesslia, Nigropogon, Nigrosphaeria, Nigrospora, Niorma, Niptera, Nitschkea. Nodulisphaeria, Nolanea, Nomuraea, Normandina, Norrlinia, Nostotheca, Notansiella, Nothodiscus, Nothoravenelia, Nothospora, Nothostroma, Nowakowskia, Nowakowskiella, Nowellia, Nozcniia, Nummularia, Nyctalis, Nylanderiella, Nynianomyces, Nyssopsora, Nyssopsorella, Obelidium, Ocellaria, Ocellularia, Ochroechia, Ochropsora, Octaviana, Oobntia, Odontoschf, Odontotrema, Odontotrcinela, Odontura, Oedemium, Oedocephalum, Oedomyces, Ohleria, Ohleriela, Oidiopsis, Oidium, Oleina, Oleinis, Oligostroina, Olivea, Ollula, Olpidiaceae, Olpidiae, Olpidiaster, Olpdiopsis, Olpidium, Olpitrichum, Ombrophila, Omphalia, Omphalospora, Oncopodium, Oncospora, Ontotelium, Onygena, Onygenaceae, Oomyces, Oospora, Oosporidca, Qothecium, Qothedum, Opeasterina, Opeasterinela, Opegrapha, Opethyrium, Ophiobolus, Ophiocapnis, Ophiocapnodium, Ophiocarpela, Ophioceras, Ophiochaeta, Ophiocladium, Ophiodictyum, Ophiodothella, Ophiodothis, Ophiogloea, Ophiognomonia, Ophiomassaria, Ophiomeliola, Ophionectria, Ophiopeltis, Ophiosphaerella, Ophiosphaeria, Ophiostoma, Ophiostomela, Ophiotexis, Ophiotrichum, Oplothecium, Oraniella, Orbicula, Orbilia, Orbiliopsis, Orcadia, Ordonia, Orinathoidium, Orphniospora, Oropogon, Orthoscypha, Oscarbrefeldia, Ostenfeldiela, Ostreionella, Ostreium, Ostropa, Ostropae, Oswaldia, Oswaldina, Otidea, Otidella, Otthia, Otthiella, Oudemansiella, Ovularia, Oxydothis, Ozonium, Pachybasidiella, Pachybasium, Pachydiscula, Pachypatella, Pachyphiale, Pachyphloeus, Pachyrhytisma, Pachyspora, Pachytrichum, Pactilia, Paedlomyces, Paepalopsis, Paidania, Palawania, Palawaniella, Pampolysporium, Panaeolus, Pannaria, Pannariae, Panus, Papularia, Papulospora, Parabotryum, Paracapnodium, Paracesatiella, Paracudonia, Paracytospora, Paradidymella, Paradiplodia, Paralaestadia, Paramazzantia, Paranectria, Paranthostomella, Parapeltella, Parasderophoma, Parasitella, Parasphaeria, Paraspora, Parasterina, Parastigmatea, Parathalle, Paratheliae, Parathelium, Parendomyces, Parenglerula, Parmelia, Parmeliaceae, Parmeliae, Parmeliella, Parmeliopsis, Parmentaria, Parmularia, Parmulariella, Parmulina, Parmulineae, Parodella, Parodiellina, Parodiopsis, Paropsis, Paryphedria, Passalora, Passeriniella, Passerinula, Patellaria, Patellariaceae, Patellea, Patellina, Patellinae, Patellonectria, Patinella, Patouillardia, Patouillardiella, Patouillardina, Pauahia, Paulia, Paurocotylis Paxillus, Paxina, Pazschkea, Pazschkella, Peccania, Peckia, Peckiella, Pedilospora, Pellicularia, Pellionella, Pelodiscus, Peloronectria, Peltaster, Peltella, Petidea, Petidium, Petigera, Peltigeraceae, Peltigerae, Pektigeromyces, Petistroma, Peltosoma, Peltosphaeria, Peltostroma, Peltostromella, Pemphidium, Penicilliopsis, Penicillium, Peniophora, Peniophorina, Penomyces, Pentagenella, Penzigia, Perforaria, Periaster, Peribotryuin, Perichlamys, Pericladium, Pericoccis, Periconia, Periconiella, Pericystis, Peridermium, Peridoxylum, Periola, Periolopsis, Perischizum, Perisporiaceae, Perisporiales, Ierisporiella, Perisporina, Perisporiopsis, lerisporiopsis, Perisporium, Peristemma, Peristomium, Perizomatium, Penzomella, Peroneutypa, Peroneutypella, Peronoplasmopara, Peronospora, Peronosporaceae, Peronosporae, Perrotia, Perrotiella, Persooniella, Pertusaria, Pertusariae, Pestalozzia, Pestalozziella, Pestalozzina, Petasodes, Petelotia, Petractis, Petrakia, Petrakiella, Peyritschiella, Peyritschiellaceae, Peyronelia, Peziotrichum, Peziza, Pezizaceae, Pezizae, Pezizales, Pezizella, Pezizellaster, ZPezoepis, Pezoloma, Pezomela, Phacenula, Phacidiaceae, Phacidiales, Phacidiella, Phacidina, Phacidiostroma, Phacidium, Phacopsis, Phacopsora, Phaeangella, Phaeangium, Phaeapiospora, Phaeaspis, Phaeharziella, Phaeidium, Phaeisaria, Phaeisariopsis, Phaeobotryosphaeria, Phaeobotryum, Phaeocapnodinula, Phaeochora, Phaeochorella, Phaeociboria, Ihaeoclavulina, Phaeoconis, Phaeocreopsis, Phaeocryptopus, Phaeocyphella, Phaeocytostroma, Phaeoderris, Phaeodiaporthe, Phaeodimeriella, Phaeodimeris, Phaeodiscula, Phaeodomus, Phaeodothiopsis, Phaeodothis, Phaeofabraea, Phaeoglossum, Phaeographina, Phaeographis, Phacoliggrocybe, Phaeolabrella, Phaeolimacium, Phaeomacropus, Phaeomarasniius, Phaeomarsonia, Phaeomarssonia, Phaeomeris, UIhaeoiiionostichella, Phaeopeltis, Phaeopeltis, Phaeopeltium, Phaeopeltosphaeria, Phaeopezia, Phaeophacidium, Phaeophleospora, Phaeophomatospora, Phaeophomopsis, Phaeopolynema, Phaeopterula, Phaeoradulum, Phaeorhytisma, Phaeosaccardinula, Phaeoschiffnerula, Phaeoscutella, Phaeoseptoria, Phaeosperma, Phaeosphaerella, Phaeosphaeria, Phaeospora, Phaeosporis, Phaeostigme, Phaeostigme, Phaeostilbella, Phaeothrombis, Phaeotrabutiella, Phaeotrema, Phaeotremella, Phaeotrype, Phallaceae, Phallobata, Phallogaster, Phallus, Phalodictyum, Phalostauris, Phalothrix, Phanerascus, Phanerococcus, Phanerocorynelia, Phanerocorynenm, Phaneronipces, Phanosticta, Phanotylium, Pharcidia, Pharcidiella, Pharcidiopsis, Phelorina, Phellostroma, Phialea, Phialophoi-a, PhiU,psiella, Philocopra, Philonectria, Phlebia, Phlebophora, Phleboscyphus, Phlegmophiale, Phleogena, Phleospora, Phloeoconis, Phloeopeccania, Phlocophthora, Phlocosporella, Phlocosporina, Phlyctaena, Phlyctaeniella, Phlyctella, Phlyctidia, Phlyctidium, Phlyctis, Phlyctochytrium, riioenicostronia, Pholiota, Pholiotella, Phoma, Phomaceae, Phomachora, Phomales, Phomatospora, Phomatosporopsis, Phomopsina, Phomopsis, Phomyces, Phorcys, Phragmidiella, Phragmidium, Phragmocalosphaeria, Phragmocapnias, Phragmocarpella, Phraginocauma, Phragmodochium, Phragmodothella, Phragmodothidea, Phragmodothis, Phragmonaevia, Phragmopeltis, Phragmopyxine, Phragmopyxis, Phragmoscutella, Phragmosperma, Phragniotelium, Phragmothee, Phragmothyriella, Phragmothyrium, Phragmotrichum, Phthora, Phycascus, Phycodiscis, Phycomyces, Phycomycetes, Phycopsis, Phyllachora, Phyllachorae, PhylAachorella, Phyllactinia, Phylliscidium, Phylliscum, Phyllobathelium, Phylloblastia, Phyllobrassia, Phyllocarbon, Phyllocelis, PhylAocelis, Phyllocrea, Phylloedia, Phyllomyces, Phyllonochaeta, Phyllophthalmaria Phylloporna, Phylloporis, PhylAoporthe, Phylloporus, Phyllopsora, Phyllopsorae, PhylAosticta, PhylAostictina, Phyllotremella, Phymatodiscus, Phymatosphaeria, Phymatotrichum, Physalacria, Physalospora, Physalosporella, Physalosporina, Physcia, Physciaceae, Physcidia, Physma, Physmatomyces, Physoderma, Physopella, Physospora, Physosporella, Phytophthora, Pichia, Picoa, Piersonia, Piggotia, Pila, Pilacre, Pilacrella, Pilaira, Pileolaria, Pilgeriella, Pilidiella, Pilidium, Piline, Pilobolae, Pilobolus, Pilocratera, Pilophorum, Pilosace, Pilula, Piniina, Pinoyella, Pionnotes, Piptocephalis, Piptostoma, Piptostomum, Pirella, Piricauda, Piricularia, Piringa, Pirobasidium, Pirogaster, Pirostoma, Pirostomella, Pirostomella, Pirottaea, Pisolithus, Pisomyxa, Pistillaria, Pithomyces, Pitya, Pityella, Placasterella, Placidiopsis, Placodiplodia, Placodothis, Placographa, Placonema, Placonemina, Placopeziza, Placophomopsis, Placosoma, Placosphaerella, Placosphaeria, Placostroma, Placothelium, Placothyrium, Plactogene, Ilacuntium, Placynthium, Plaiorhabdus, Plagiostigme, riagiostoma, Ilagiostomella, Magiostroniella, Ilagiotrema, Plasmodiophora, Plasmodiophoraceae, Plasmopara, Plasmophagus, liatycarpiun; Platychora, Platygloea, riatypcltella, Ilatysticta, Platystomum, Plearthonis, Plectania, Plectodiscella, Plectonaemella, Plectopeltis, Plectophoma, Plectophomella, Plectophomopsis, Plectosira, Plectosphaera, Plectosphaerella, Plectospira, Plectothrix, Plenodomus, Plenophysa, Plenotrichum, Plenozythia, Pleochaeta, Pleochroma, ileococcum, Pleoconis, Pleocouturea, Pieocyta, Pleodothis, Pleogibberella, Pleoglonis, Pleolecis, Pleolpidium, Pleomassaria, Pleomeliola, Pleomelogramma, Ileomeris, Pleomerium, Pleonectria, Pleopatella, Pleophalis, Pleophragrma, Pleopyrenis, Pleoravenelia, Pleorinis, Pleoscutula, Pleosphaeria, Pleosphaeropsis, Pleosphaeropsis, Pleosphaerulina, Pleospilis, Pleospora, Pleosporopsis, Pleostictis, Pleostomella, Pleotrachelus, Plcurage, Pleurascus, Pleuroceras, Pleurocolla, Pleurocybe, Pleurocytospora, Pleurodiscula, Pleuronaema, Pleurophoma, Pleurophomella, Pleurophomopsis, Pleuroplaconema, Pleuroplacosphaeria, Pleurostoma, Pleurostomella, Pieurothecium, Pleurotheliopsis, Pleurothyriella, Pleurothyrium, Pleurotrema, Pleurotus, Plicaria, PHcariella, Plochmopeltideila, Plochmopeltineae, Plochmopeltis, Ploettnera, Plowrightia, Plownghtiella, Ilunporus, Pluteolus, Pluteus, Pocillum, Pocosphaeria, Podaleuris, Podaxon, Podocapsa, Podocapsium, Podochytrium, Podocrea, Podonectria, Podophacidium, Podoplaconema, Podosordaria, Podosphaera, Podospora, Podosporiella, Podosporium, Podostictina, Podostroma, Podostroma, Podoxyphium, Poecilosporium, Polhysterium, Polioma, Poliomella, Poliotelium, Polyascomyces, Polyblastia, Polyblastiopsis, Polycarpella, Polyychaetella, Polychaetum, Polychaetum, Polychidium, Polyclypeolum, Polycoccum, Polycyclina, Pdycyclus, Polydesmus, Polygaster, Poylagenochromatia, Polymorphomyccs, Polynema, Polyopeus, Polyphagus, Polyplocium, Polyporaceae, Polyporus, olyrhina, Polyrhizum, Polysaccopsis, Polysaccum, Polyscytalum, Polyspora, Polysporidium, Polystictus, Polystlgma, Polystigmina, Polystomella, Polystomellaceae, Polystomelleae, Polystroma, Polythelis, Polythelis, Polythrincium, Polythyrium, Polytrichia, Pompholyx, Poria, Porina, Porinopsis, Porocyphus, Poronia, Poropeltis, Poroptyche, Porostigme, Porothelium, Porphyrosoma, Porterula, Pragmopara, Preussia, Prilleuxia, Prilleuxina, Pringsheimia, Prismaria, Pritzeliella, Proabsidia, Prodisea, Promycetes, Pronectria, Prophytroma, Propoldium, Propolina, Propoliopsis, Propolis, Prospodium, Prosthecium, Prosthemiella, Prosthemium, Protascus, Protasia, Proteomyces, Protoachlya, Protoblastenia, Protocalicium, Protococcales, Protocoronis, Protocoronospora, Protodontia, Protoglossum, Protohydnum, Protomerulius, Protomyces, Protomycetaceae, Protomycopsis, Protopeltis, Protoscypha, Protoscypha, Protostegia, Protothyrium, Protoventuria, Protubera, Psalidosperma, Psalliota, Psammina, Psathyra, Psathyrella, Pseudacolium, Pseuderiospora, Pseudoabsidia, Pseudobalsamia, Pseudobeltrania, Pseudocamptoum, Pseudocenangium, Pseudocercospora, Pseudocytospora, Pseudodiaporthe, Pseudodichomera, Pseudodictya, Pseudodimerium, Pseudodimeriujn, Pseudodiplodia, Pseudodiscosia, Pseudodiscula, Pseudofumago, Pseudogaster, Pseudogenea, Pseudographis, Pseudographium, Pseudoguignardia, Pseudohaplis, Pseudohaplosporella, Pseudohelotium, Pseudoheppia, Pseudohydnotrya, Pseudolachnea, Pseudolecanactis, Pseuoblembosia, Pseudolizonia, Pseudolpidiopsis, Pseudolpidium, Pseudomassaria, Pseudombrophila, Pseiidomelasniia, Pseudomeliola, Pseudomicrocera, Pseudomonilia, Pseudomycoderma, Pseudonectria, Pseudoparmelia, Pseudoparodia, Pseuobparodiella, Pseudopatella, Pseudopatellina, Pseudoperis, Pseudoperisporium, Pseudoperonospora, Pseudopeziza, Pseudophacidium, Pseudophoma, Pseudophomopsis, Pseudophyllachora, Pseudophysalospora, Pseudopityella, Pseudoplasmopara, Pseudoplea, Pseudoplea, Pseudoplectania, Pseudopleospora, Pseudopoystgimina, Pseudopuccinia, Pseudopyrenula, Pseudorhynchia, Pseudorhytisma, Pseudosaccharomyces, Pseudosclerophoma, Pseudoseptoria, Pseudosphaerella, Pseudosphaeria, Pseudostegia, Pseudostictis, Pseudothiopsella, Pseudothis, Pseudothyridaria, Pseudotrochila, Pseudotryblidium, Pseudotrype, Pseudotthia, Pseudotthiella, Pseudovalsa, Pseudovularia, Pseudozythia, Psilocybe, Psiloglonium, Psilonia, Psilopezia, Psilospora, Psilosporina, Psilothecium, Psora, Psorella, Psoroglaena, Psorographis, Psoroma, Psoromaria, Psorotheciella, Psorotheciopsis, Psorotichia, Psyllidomyces, Pteridiospora, Pteromyces, PterophylAus, Pterula, Pterygiopsis, Pterygium, Ptychographa, Ptychopeltis, Puccinia, Pucciniaceae, Pucciniales, Pucciniastrum, Pucciniopsis, Pucciniosira, Pucciniospora, Pucciniostele, Puiggariella, Puiggarina, Pullularia, Pulparia, Pulveraria, Punctillum, Pustularia, Puttemannsia, Puttemannsiella, Pycnidiella, Pycnidiostroma, Pycnis, Pycnocarpum, Pycnochytrium, Pycnoderma, Pycnodothis, Pycnographa, Pycnomma, Pycnopeltis, Pycnosporium, Pycnostemma, Pycnostroma, Pycnostysanus, Pycnothyrium, Pyrertastrum, Pyrenidiae, Pyrenidium, Pyreniella, Pyrenobotrys, Pyrenochaeta, Pyrenochaetina, Pyrenocollema, Pyrenodiscus, Pyrenomyxa, Pyrenopezis, Pyrenopeziza, Pyrenopezizae, Pyrenopezizopsis, Pyrenophora, Pyrenopoyporus, Pyrenopsidae, Pyrenopsidium, Pyrenopsis, Pyrenostigme, Pyrenothamnia, Pyrenotheca, Pyrenothrix, Pyrenotrichum, Pyrenotrochila, Pyrenula, Pyrenulae, Pyrenyllium, Pyrgidium, Pyrgillus, Pyrhosorus, Pyronema, Pyronemella, Pythiae, Pythiocystis, Pythiogeton, Pythiomorpha, Pythiopsis, Pythium, Pyxidiophora, Pyxine, Quatemaria, Queletia, Questiera, Rabenhorstia, Rachisia, Raciborskiella, Kaciborskioiiyces, Racodium, Radaisella, Radulum, Ramalina. Ramalodium, Ramonia, Ramosiella, Ramsbottomia, Ramularia, Ramulariopsis, Raniulariospora, Ramularisphaerclla, Ramulaspera, Rainulispora, Ranojevicia, Ravenelia, Ravenelula, Readerella, Rebentischia, Reessia, Rehniella, Rehmiellopsis, Rehmiodothis, Rehmiomyces, Reinkella, “IC Resticularia, Reyesiella, Rhabdium, Rhabdocline, Rhabdogloeopsis, Rhabdogloeum, Rhabdopsora, Rhabdospora, Rhabdostroma, Rhabdostromella, Rhabdostromellina, Rhabdostromina, Rhabdothyrella, Rhabdothyrium, Rhachomyces, Rhacodiella, Rhacodium, Rhacophyllus, Rhadinomyces, Rhagadolobium, Rhagadostoma, Rhamphoria, Rhamphospora, Rhaphidisegestria, Rhaphidocyrtis, Rhaphidophora, Rhaphidopyris, Rhaphidospora, Rhaphidvlis, Rheumatopeltis, Rhinocladium, Rhinotrichum, Rhipidium, Rhipidocarpum, Rhizalia, Rhizidiocystis, Rhizidiomyces, Rhizidium, Rhizina, Rhizinae, Rhizocalyx, Rhizocarpum, Rhizoclosmatium, Rhizoctonia, Rhizogene, Rhizohypha, Rhizomorpha, Rhizomyces, Rhizomyxa, Rhizophidium, Rhizophlyctis, Rhizophoma, Rhizopogon, Rhizopus, Rhizosphaera, Rhizosphaerella, Rhizotexis, Rhizothyrium, Rhodobolites, Rhodochytrium, Rhodocybe, Rhodomyces, Rhodopaxillus, Rhodoseptoria, Rhodosticta, Rhodothrix, Rhodotorula, Rhodotus, Rhombostilbella, Rhopalidium, Rhopalocystis, Rhopalomyces, Rhopographella, Rhopographina, Rhopographus, Rhymbocarpus, Rhynchodiplodia, Rhynchomelas, Rhynchomeliola, Rhynchomyces, Rhynchomyces, Rhynchonectria, Rhynchophoma, Rhyncophoromyces, Rhynchophorus, Rhynchosphaeria, Rhynchosporium, Rhynchostoma, Rhynchostomopsis, Rhyparobius, Rhysotheca, Rhytidenglerula, Rhytidhysterium, Rhytidopeziza, Rhytisma, Rhytismella, Riccoa, Richonia, Rickia, Rickiella, Riessia, Rimbachia, Rinia, Rinodina, Robergea, Robertomyces, Robillardia, Robledia, Roccella, Roccellae, Roccellaria, Roccelina, Roccellographa, Rodwaya, Roesleria, Roestelia, Rollandina, Romellia, Rosellinia, Rosenscheldia, Rosenscheldiella, Rostkovites. Rostrella, Rostronitschkea, Rostrosphaeria, Rostrupia, Rotaea, Rotularia, Roumegueria, Roumegueriella, Roussoella, Rozella, Rozites, Ruhlandlella, Russula, Rutstroemia, Sabourauditcs, Saccardaea, Saccardia, Saccardiae, Saccardinula, Saccardoella, Saccardomyces, Saccharomyces, Saccharomycetaceae, Saccharomycodes, Saccharomycopsis, Saccoblastia, Saccobolus, Saccomyces, Saccothecium, Sachsia, Saddium, Sagediopsis, Sagiolechia, Saitomyces, Samarospora, Sampaioa, Santiella, Saprolegnia, Saprolegniaceae, Saprolegniae, Sapromyces, Sarcinella, Sarcinodochium, Sarcinomyces, Sarcographa, Sarcographina, Sarcomyces, Sarcophoma, Sarcopodium, Sarcopyrenia, Sarcoscypha, Sarcosphaera, Sarcosoma, Sarcotrochila, Sarcoxylum, Sarophorum, Sartorya, Scaphidium, Scelobelonium, Scenomyces, Sceptromyces, Schenckiella, Schiffnerula, Schinlia, Scdiinzinia, Schismatomma, Schistoods, Schistophorum, Schizachora, Schizacrospernnim, Schizocapnodium, Schizonella, Schizoparme, Schizopelte, Schizophyllum, Schizosaccharis, Schizosaccharomyces, Schizospora, Schizostege, Schizostoma, Schizothyrella, Schizothyrioma, Schizothyrium, Schizotrichum, Schizoxyum, Schneepia, Schoenbornia, Schroeterella, Schroeteria, Schroeteriaster, Schulzeria, Schwanniomyces, Schweinitziella, Sdodothis, Scirrhia, Scirrhiachora, Sdrrhiella, Sdrrhiopsis, Sdrrhodothis, Scirrhophragma, Sclerangium, Sclerochaeta, Sclerochaetella, Sderococcum, Scderocystis, Sclerodcpsis, Sderoderma, Sceroderris, Scderodiscus, Sderodothiorella, Sclerodothis, Sderographis, Sderographium, Scleromeris, Sderophoma, Sclerophomella, Sclerophomina, Sderophytum, Sderoplea, Sderoplella, Sderopycnium, Sclerosphaeropsis, Sclerospora, Sderostagonospora, Sderotelium, Sderotheca, Sclerothyrium, Sclerotinia, Sderotiomyces, Sderotiopsis, Sderotium, Scodellina, Scolecactis, Scoleciocarpus, Scolecobasis, Scolecoccoidea, Scolecodothis, Scolecodothopsis, Scoleconectria, Scolecopeltidella, Scolecopeltidium, Scolecopeltis, Scolecopeltium, Scolecopeltopsis, Scolecosporiella, Scolecotrichum, Scolecozythia, Scoliciosporium, Scolionema, Scopinella, Scopophoma, Scoptria, Scopularia, Scopulariopsis, Scorias, Scoriomyces, Scortechinia, Scutelinia, Scutellum, Scutula, Scutularia, Scutehinia, Scuteliniae, Scyphospora, Scyphostroma, Scytopezis, Sebacina, Secotium, Seismosarca, Selenophoma, Selenophomopsis, Selenotila, Selinia, Semigyalecta, Sepedonium, Septobasidium, Septochora, Septocladia, Septocylindrium, Septocyta, Septocytela, Septodothideopsis, Septogloeum, Septoideum, Septomazzantia, Septomyxa, Septonema, Septopatella, Septorella, Septona, Septoriella, Septoriopsis, Septorisphaerella, Septosporium, Septothyrella, Septotrullula, Sepultaria, Setchellia, Setella, Seuratia, Seynesia, Seynesiola, Seynesiopsis, Shearia, Shiraia, Shropshiria, Sigmatomyces, Simoidomyces, Sillia, Simblum, Simonyella, Siphonaria, Siphula, Sirentyloma, Sirexcipula, Sirexcipulina, Siridiella, Siridina, Siridium, Sirobasidium, Sirococcus Sirocyphis Sirodesmium, Sirodiplospora, Sirodochiela, Sirodothis, Sirogloea, Sirolegniella, Sirolpidium, Siropatela, Sirophoma, Siroplaconema, Siroplaconema, Siroscyphela, Siroscyphellina, Sirosperma, Sirosphaera, Sirospora, Sirosporium, Sirostromella, Sirothecium, Sirothyriela, Sirothyrium, Sirozythia, Sirozythiella, Sistotrema, Skepperia, Skepperiela, Skierkia, Skottsbergiella, Smeringomyces, Solanella, Solenia, Solenodonta, Solenoplea, Solenopsora, Solorina, Solorinella, Sommerstorffia, Sordaria, Sorica, Sorodiscus, Sorokinia, Sorolpidium, Sorosphaera, Sorosporium, Sorothelia, Sparassis, Spathularia, Spegazzinia, Spegazzinula, Spermatoloncha, Spennodennia, Spennophthora, Sphacelia, Sphaceliopsis, Sphacelotheca, Sphaerella, Sphaerellothedum, Sphaeriaceae, Sphaeriales, Sphaericeps, Sphaeridium, Sphaeriostromella, Sphaeriothyrium, Sphaerita, Sphaerobolus, Sphaerocista, Sphaerocolla, Sphaerocreas, Sphaeroderma, so Sphaerodermella, Sphaerodes, Sphaerodothis, Sphaerognomonia, Sphaerographium, Sphaeromyces, Sphaeronema, Sphacronemella, Sphaeronemina, Sphaeronemopsis, Sphaeropezia, Sphaerophoma, Sphaerophoropsis, Sphaerophorus, Sphaerophragmium, Sphaeropsis, Sphaerosoma, Sphaerospora, Sphaerosporium, Sphaerostilbe, Sphaerostilbella, Sphaerotheca, Sphaerothyrium, Sphaerulina, Sphaleromyces. Spheconisca, Sphenospora, Sphinctrina, Sphinctrinopsis. Spicaria, Spicularia, Spilodochium, Spilomium, Spilomyces, Spilonema, Spilopezis, Spilopodia, Spilosticta, Spinalia, Spinellus, Spira, Spiralia, Spirechina, Spirogramma, Spirographa, Spirogyrales, Spirospora, Spolverinia, Spondylocladium, Spongospora, Sporendonema, Sporhelminthiunm Sporobolomyces, Sporoclema, Sporoctclmorpha, Sporocybe, Sporocystis, Sporoderma, Sporodesmium, Sporodictyum, Sporodinia, Sporodiniopsis, Sporomega, Sporomyxa, Sporonema, Sporophlyctis, Sporophysa, Sporopodium, Sporormia, Sporormiella, Sporoschisma, Sporostachys, Sporotrichella, Sporotrichum, Spragueola, Spumatoria, Squamotubera, Stachybotryella, Stachybotrys, Stachylidium, Stagonopatela, Stagonopsis, Stagonospora, Stagonosporopsis, Stagonostroma, Stagonostromela, Staheliomyces, Stalagmites, Stamnaria, Starbaeckia, Starbaeckiella, Staurochaeta, Stauronema, Staurophoma, Staurothele, Steganopycnis, Steganosporium, Stegasphaeria, Stegastroma, Stegia, Stegopeziza, Stegopezizella, Stegophora, Stegothyrium, Steinera, Stella, Stemmaria, Stemphyliomma, Stemphyliopsis, Stemphyliopsis, Stemphylium, Stenocarpela, Stenocybe, Stephanoma, Stephanospora, Stephanotheca, Stephensia, Stereocaulum, Stereochlamys, Stereocrea, Stereolachnea, Stereostratum, Stereum, Sterigmatocystis, Stevensea, Stevensiela, Stevensula, Stichodothis, Stichomyces, Stichopsora, Stichospora, Stictata, Stictae, Stictidaceae, Stictina, Stictinae, Stictis, Stictochorela, Stictochorellina, Stictoclypeolum, Stictopatela, Stictophacidium, Stictostroma, Stigeosporium, Stigmatea, Stigmateae, Stigmatella, Stigmatodothis, Stigmatomyces, Stigmatopeltis, Stigmatophragmia, Stigmatopsis, Stigme, Sigmella, Stigmina, Stigmochora, Sigmopeltella, Zld Stigmopeltis, Stigmopsis, Stilbaceae, Stilbella, Stilbochalara, Stilbocrea, Stilbodendrum, StilbohypoxyIon, Stilbomyces, Stilbonectria, Stilbopeziza, Stilbospora, Stilbothamnium, Stilbum, Stirochaete, Stomatogene, Stomiopeltela, Stomiopeltis, Strasseria, Streptotheca, Streptothnx, Strickeria, Strigula, Strigulae, Strobilomyces, Stromatrma, Stromatographium, Stroinatostysanus, troninc, Stropharia, Strossmayera, Strumela, Strumellopsis, Stuartcla, Stylina, Stylobatcs, Stylonectria, Stypella, Stypinella, Stysanopsis, Stysanus, Subiilariella, Subulicola, Succinaria, Suiliis, Sydowia, Sydowiella, Sydowina, Sydowinula, Symphaeophyma, Symphaster, Symphyosira, Symplectromyces, Synalissa, Synarthonia, Syncarpella, Syncephalastrum, Syncephalidae, Syncephalis, Synchactophagus, Synchytriaceae, Synchytrium, Syncsiella, Synesiopeltis, Synglonium, Synnematium, Synomyces, Synostomella, Synpeltis, Synsporium, Syntexis, Synthctospora, Systremma, Systrcmmopsis, Syzygitcs, Taeniophora, Tangella, Tapellaria, Tapesia, Taphridium, Taphrina, Tarichiun, Tarzetta, Tassia, Teichospora, Teichosporella, Telcutospora, Telimena, Tcloconia, Tclospora, Tcphrosticta, reratomyces, Teratonema, Teratosperma, Teratosphaeria, Terfezia, Terfeziopsis, Termitaria, Testiculana, Testudina, Tetrachia, Tetrachytriuin, Tetracium, Tetracladium, Tetracoccosporis, Tetracoccosporium, Tetramyxa, Tetraploa, Thalassoascus, Tlialassomyces, Thallochaete, Thalloedema, Thamnidium, Thamnocephalis, Thamnolia, Thamnomyces, Thaxteria, Thaxteriella, Thecaphora, Theciopcltis, Thecopsora, Thecostroma, Thecotheus, Theissenia, Theissenula, Thelebolus, Thelenidia, Thelephora, Thelephoraceae, Thelidiopsis, Thelidium, Thelis, Thelocarpum, Theloporus, Thelopsis, Theloschistes, Thelospora, Thelotrema, Thermoidium, Themion yccs, Thermutis, Therrya, Thielavia, Thielaviopsis, Tholuma, Thoracella, Thozetia, Thrauste, Thraustotheca, Thrombium. Thuemenella, Thwaitesiella, Thyrea, Thyriascus, Thyridaria, Thyridella, Thyridium, Thyrinula, Thyriopsis, Thyriostoma, Thyriostroiiia, Thyrococciim, Thyrodochium, Thyronectria, Thyronectroidea, Thyrosoma, Thyrospora, Thyrostroma, Thyrostromella, Thyrsidiella, Thyrsidina, Thyrsidium, Thysanopyxis, Thysanothecium, Tiarospora, Tiarosporella, Tichospora, Tichosporella, Tichothecium, Tieeheniella, TilachlidioDsis, Tilachlidium, Tilletia, Tilletiaceae, Tilotus, Tirmania, Titaea, Titaeospora, Titaeosporina, Titanella, Titania, Tibodasia, Togninia, Tolypomyria, Tolyposporella, Tolyposporium, Tomasiella, Tomentellina, Tonduzia, Toninia, Topospora, Torrendia, lorrcndiella, Torrubiella, Torsellia, Torula, Torula, Torulina, Toruloidea, Torulopsis, Torulospora, Toxosporium, Trabutiella, Trachysphaera, Trachyspora, Tracbysporella, Trachythyriolum, Trachyxylaria, Tracya, Tracyella, Trailia, Trailia, Trametes, Tranzschelia, Traversoa, Treleasia, Treleasiella, Trematophoma, Trematosphaerella, Trematosphaeria, Trematosphaeriopsis, Trematosphaeris, Treinatovalsa, Tremella, Tremellaceae, Tremellales, Tremellidium, Tremellodendrum, Tremellodon, Tremellogaster, Tremellopsis, Tremotylium, Treubiomyces, Triactella, Tricella, Trichaegum, Trichaleurina, Trichaleuris, Tricharia, Tricharia, Trichaster, Trichasterina, Trichobacidia, Trichobelonium, Trichobotrys, Trichochora, Trichococcinus, Trichocladium, Trichocollonema, Trichocoma, Trichoconis, Trichocrea, Trichoderma, Trichodiscula, Trichodochium, Trichodothis, Trichodytes, Trichofusarium, Trichoglossum, Trichohleria, Tricholoma, Trichomerium, Trichonectria, Trichopelteae, Trichopeltella, Trichopeltina, Trichopeltis, Trichopeltium, Trichopeltopsis, Trichopeltula, Trichopeltulum, Trichophila, Trichophyma, Trichophytum, Trichopsora, Trichoscypha, Trichoseptoria, Trichosperma, Trichospermella, Trichosphaerella, Trichosphaeria, Trichosporina, Trichosporium, Trichosterigma, Trichostronia, Trichothallus, Tricliotheca, Trichothecium, Trichothelium, Trichothyriaceae, Trichothyriella, Trichothyriopsis, Trichothyrium, Trichotrema, Trichurus, Tridens, Trigphium, Trigonosporium, Trimmatostroma, Trimmatothele, Trinacrium, Tiphragmiopsis, Tiphragmium, Triplicaria, Tipospermum, Tripospora, Tiposporina, Triposporium, Trochila, Trochodium, Trogia, Tromcra, Troposporella, Troposporium, i Trotteria, Trotterula, Trullula, Tryblidaria, Tryblidiaceae, Tryblidiella, Tryblidiopsis, Tryblidiopycnis, Tryblidis, Tryblidium, Tryblis, Trypetheliae, Trypethelium, Tubaria, Tuber, Tuberaceae, Tuberales, Tubercularia, Tuberculariaceae, Tiibercularielia, Tiiberculariopsis, Tubercularis, Tuberculna, Tuberculis, Tubeufla, Tuburcinia, Tulasnella, Tylophilus, Tylophorella, Tylophorum, Tylostoma, Tympanis, Tympanopsis, Typhula, Typhulochaeta, Tyridiomyces, U Ulcodollella, Ulcodothis, Uleomyces, Uleopeltis, Uleothyrium, Ulocodla, Umbilicaria, Uncigera, Uncinula, Underwoodia, Unguicularia, Unguiculariopsis, Uredinopsis, Uredo, Umula, Urobasidium, Uroconis, Urocystis, Lrohcndersonia, Uromyces, Uromycladium, Uromycopsis, Urophiala, Urophlyctis, Uropolystigma, Uropyxis, Urospora, Urosporella, Urosponium, Usnea, Usneae, Ustilaginaceae, Ustilaginales, Ustilaginodes, Ustilago, Ustilagopsis, Ustulina, Valdensia, Valetoniella, Valsa, Valsaria, Valsella, Valseutypella, Valsonectria, Vanderystiella, Varicellaria, Varicosporium, Vasculomyces, Vaucheriales, yi Veloziella, Velutaria, Venturia, Venturiella, Vermicularia, Vermiculariella, Verpa, Verrucaria, Verrucariaceae, Verrucariae, Verrucaster, Vertidcladium, Verticilliae, Verticillidochium, Verticilliopsis, Verticillis, Verticillium, Vestergrenia, Vialaea, Vibrissea, Virgaria, Vittadinula, Vivianella, Vizella, Voeltzknowiella, Vokartia, Volutena, Voutellaria, Voutelis, Voutellopsis, Volutellops!s, Volutina, Volvaria, Volvariella, Volvoboletus, Vouauxiella, Wageria, Wallrothiella, Wardina, Wardomyces, Wawelia, Wecsea, Wegelina, Weinmannodora, Wentiomyces, Wettsteinina, Wiesnerina, Wiesneriomyces, Willeya, Williopsis, Winterella, Winterina, Winteromyces, Wojnowicia, Wolkia, Woodiella, Woronina, Woroninae, Woroninella, Wynnea, Mynnella, Xanthocarpia, Xanthopsora, Xanthopyrenia, Xanthoria, Xenodochus, Xenodomus, Xenogloea, Xenolophium, Xenomeris, Xenomyces, Xenonectna, Xenopeltis, Xenopus, Xenosphaeria, Xenosporella, Xenosporium, Xenostee, Xenostroma, Xenothedum, Xerotus, Xiphomyces, Xylaria, Xylariodiscus, Xylobotryum, Xyloceras, Xylocladium, Xylocrea, Xyloglyphis, Xylogramma, Xylographa, Xyloma, Xylopodium, Xyloschistes, Xyloscbizuin, Xylostroma, Xystozukalia, Yatesula, Yoshinagaia, Yoshinagamyces, Yoshinagella, Ypsilonia, Zaghouania, Zahlbrucknerella, Zignoella, Zimmermanniella, Zodiomyces, Zonosporis, Zoophagus, Zopfla, Zopfiella, Zukalia, Zukalina, Zukaliopsis, Zukaliopsis, Zygochytrium, Zygodesmella, Zygodesmus, Zygorhizidium, Zygosaccharis, Zygosaccharomyces, Zygosporium, Zythia and Zythiaceae.

### Further Embodiments

The present invention includes delivering skopobiota to soil, including skopobiota which comprises any of the microbiota described herein. The invention includes delivery of skopobiota to soil to prevent and/or treat infection by Pythium violae and/or Phytophthora infestans and/or Fusarium oxysporum and/or Rhizoctonia solani and/or Streptomyces scabies, which are of major concern in the soils of many rich horticultural crops. The present invention, and in particular this embodiment, may prevent and/or treat infection of a crop, such as Brassica, carrots, potato, tomato, cereals or onions.

Bentonite

Bentonite is a mineral absorbent aluminium phyllosilicate clay consisting mostly of montmorillonite. It is frequently used in clarifying juice and wines (both white and red). For example, it is often used by the winemaking industry to remove proteins and other undesirable components from wines in a process known as bentonite fining—It functions as a cation exchanger. It finds many other applications ranging from medical or cosmetic products, such as treating rashes and acne or as a hair mask, it may be added to foods or drinks with the aim of relieving digestive issues or removing toxins from the body. Addition of bentonite to the soil (in our case in a very low amount, v/v) will cause no harm, neither to the environment nor to the soil microbiota and plant roots. In a preferred embodiment, the bentonite selected comprised calcium bentonite.

Biochar

Biochar is a high-carbon, fine-grained residue that today is produced through modem pyrolysis processes; it is the direct thermal decomposition of biomass in the absence of oxygen (preventing combustion), which produces a mixture of solids (the biochar proper), liquid (bio-oil), and gas (syngas) products. It is used as a soil amendment for both carbon sequestration and soil health benefits. It may resuscitate soils that have been depleted by industrial agriculture.

Biochar has not been previously associated with the skopobiota.

Biochar compositions and effects on soil fertility vary widely depending on the biological material used as substrate for its preparation and on the industrial process of its production.

In a preferred embodiment, the biochar selected was prepared from Acacia tree wood.

An example of this may be obtained from Ibero Massa Florestal, S.A.

It had the following physicochemical properties.

Fixed Carbon≥95% (EN 1860-2) Moisture≤2% (EN 1860-2)

Ash<2% (EN 1860-2) Volatile matter≤3% (EN 1860-2)

Calorific value≥8000 Kcal/Kg Fluoranthene<0.02 mg/kg (m.s.)

Sum. I-TEQ (PCDD/F+PCBs Dioxin-like)<0.79 μg/g Pyrene<0.02 mg/kg (m.s.) Sum of PCB28, PCB52, PCB101, PCB138, PCB 153 e PCB180<590 μg/g Benzo(a)anthracene<0.02 mg/kg (m.s.) Sum of Dioxin (PCDD/F-TEQ-OMS)<0.06 pa/g Chrysene<0.02 mg/kg (m.s.)

Naphthalene 0.09 mg/kg (m.s.) Benzo(b)fluoranthene<0.02 mg/kg (m.s.)

Acenaphthylene<0.02 mg/kg (m.s.) Benzo(k)fluoranthene<0.02 mg/kg (m.s.)

Acenaphthen<0.02 mg/kg (m.s.) Benzo(a)pyrene<0.02 mg/kg (m.s.)

Fluorene<0.02 mg/kg (m.s.) Indeno(1,2,3-cd)pyrene<0.02 mg/kg (m.s.)

Phenanthrene<0.02 mg/kg (m.s) Dibenz(a,h)anthracene<0.02 mg/kg (m.s.)

Anthracene<0.02 mg/kg (m.s.) Benzo(g,h,i)perylene<0.02 mg/kg (m.s.)

These are only one example of biochars. Other embodiments may be produced from other biological material such as plants, organic waste, etc. In preferred embodiment, the selected biochar made be produced from algae in particular multicellular algae.

Bentochar

In one embodiment, it comprises a biochar in particular a powdery biochar (instead of common biochars which are produced as pellets). Bentonite was employed to ‘glue together’ the biochar dust, creating a compound which may be inoculated with the skopobiota. Therefore, bentochar was developed with the aim of greatly increasing the biochar active surface and control its porosity as well. As suggested by the results obtained, bentochar is a porous material which promotes plant growth and at the same time constitutes a suitable material to deliver the skopobiota to the soil, in a way that releases the microorganisms uniformly in a large volume of soil and in a slow, gradual manner.

Consequently, bentochar promotes the growth of plants, is not harmful to them and enhances the effects of the microorganisms which comprise the skopobiota.

Production of Bentochar (a Mixture of Biochar and Bentonite)

Multiple ‘formulations’ of bentochar (e.g. different types of biochar, different v/v ratios of bentonite/biochar) may be prepared. The following example is provided: A bentochar paste was produced by mixing volumes of bentonite, biochar dust and water in a ration of (1:2.6:1). The paste was manually molded in spheres of different volumes (<8 mm3) and dried in the oven (50° C.) overnight. FIG. 27 shows bentochar spheres.

Production of Two Fungal Skopobiota (SK-1 and SK-2)

Pure fungal cultures were grown in potato dextrose agar medium, at 25° C., in the dark. Conidia suspensions were produced as described in (Del Frari G, Gobbi A, Aggerbeck M R, Oliveira H, Hansen L H and Ferreira R B (2019) Fungicides and the grapevine wood mycobiome: a case study on tracheomycotic ascomycete Phaeomoniella chlamyobspora reveals potential for two novel control strategies. Front. Plant Sci. 10:1405. doi: 10.3389/fpls.2019.01405).

Two skopobiotas, SK-1 and SK-2, were produced by blending, in a water-based medium, an exact amount of conidia of four known fungal species.

SK-1 was composed by the following fungal species:
Cladosporium sp. strain CLO2
Penicillium sp. strain P01
Trichoderma harzianum strain CBS 121699
Alternaria infectoria strain 011

The concentration of conidia of each fungus was 1×105conidia/mL in the final skopobiota composition.

SK-2 was composed by the following fungal species:
Alternaria sp. strain A001
Aureobasidium sp. strain Au01
Didymella glomerata strain 005
Setophaeosphaeria citri strain 001

The concentration of conidia of each fungus was 1×10⁵ conidia/mL in the final skopobiota composition.

Inoculation of Skopobiotas in Bentochar

Dried bentochar was inoculated by depositing on each sphere a droplet of skopobiota suspension. A batch of bentochar inoculated with SK-1 and another batch inoculated with SK-2 were stored, separately, at 4° C. FIG. 28 shows Skopobiota inocluation into bentochar.

The Survivability of Skopobiotas in Bentochar

The survivability of fungi inoculated in bentochar was assessed weekly. The procedure is the following:
In a glass beaker, three bentochar spheres were smashed in water, allowing conidia to be released in the medium;
An aliquot of watery suspension was sampled and diluted up to 150 times;
1 mL of diluted suspension was placed in Petri dishes amended with chloramphenicol and incubated, for 10 days, at 25° C.;
The presence of skopobiota fungi was assessed by identifying them using morphological features.

The survivability assay was repeated weekly, for 4 weeks after inoculation, revealing that (a) all fungal species that belong to SK-1 were viable, (b) at least 3 out of 4 of fungal species that belong to SK-2 were viable. FIG. 29 shows survivability assay in Petri dishes of the skopobiotas SK-1 and SK-2 fungal components.

Delivery of Skopobiota-Loaded Bentochar (SLB) to the Soil

A total of three skopobiota compositions and three densities SLB/soil were tested.

1) For SK-1, we tested two densities D2 and D3, corresponding to 4% and 8% v/v (SLB/soil);

2) For SK-2, we tested two densities D2 and D3, corresponding to 4% and 8% v/v;

3) SK-3, we tested three densities D1, D2 and D3, corresponding to 2%, 4% and 8% v/v;

SK-3 consisted in equal volumes of SK-1 and SK-2 mixed together. Therefore, SK-3 D1 was composed by SK-1 1%+SK-2 1%, SK-3 D2 by SK-1 2%+SK-2 2%, and SK-3 D3 by SK-1 4%+SK-2 4%.

The corresponding volume of SLB, for each of the previously described combinations, was added to soil and thoroughly mixed. FIG. 30 is illustrative of the blending of SLB into soil.

A Note on Trichoderma Spp.

Trichoderma species are opportunistic fungi residing primarily in soil, tree bark and on wild mushrooms. Trichoderma is capable of killing other fungi and penetrating plant roots and is commonly used as both a biofungicide and inducer of plant defence against pathogens (Mendoza-Mendoza, A., et al., Molecular dialogues between Trichoderma and roots: Role of the fungal secretome, Fungal Biology Reviews (2017), as shown in https://doi.org/10.1016/j.fbr.2017.12.001).

However, the ineffectiveness of Trichoderma gamsii on Armillaria root rot (ARR) in peach trees has been documented (Schnabel, G., Rollins, A. P., and Henderson, G. W. 2011. Field evaluation of Trichoderma spp. for control of Armillaria root rot of peach. Online. Plant Health Progress. doi:10.1094/PHP-2011-1129-01-RS).

Recent findings point to the involvement of certain components of the Trichoderma secretome in inducing susceptibility to fungal attack in host plants (Lamdan N L, Shalaby S, Ziv T, Kenerley C M, Horwitz B A (2015) Secretome of Trichoderma interacting with maize roots: role in induced systemic resistance. Molecular & Cellular Proteomics 14: 1054-1063 http://www.mcponline.org).

Two recent findings that merit special mention are that some of the “elicitor-like” proteins actually suppress plant defence to facilitate access of Trichoderma to root apoplast, and the inheritable nature of Trichoderma-induced “priming” across generations of plants. Trichoderma-plant interaction is quite unique in being neither a typical symbiotic one, like mycorrhiza, nor a pathogenic one, like Fusarium; this relationship is probably still evolving towards typical symbiosis. There is a dialogue between Trichoderma and plants at the root zone that leads to mutual benefits. At this time the receptor for the “elicitor^{M} proteins has not been identified (with the exception of EIX). Possible mechanisms include these proteins migrating inside the plants. However clearly the intricacies of Trichoderma-plant interactions lead to growth promotion and induced immunity, boosting agricultural growth (Mendoza-Mendoza, A., et al., Molecular dialogues between Trichoderma and roots: Role of the fungal secretome, Fungal Biology Reviews (2017), https://doi.org/10.1016/j.fbr.2017.12.001).

Effects on the Growth of Nine-Week-Old Carrots

Twelve carrot seeds cv Nantes were sown in soil containing the different combinations of SLB described above, as well as in positive and negative controls, for a total of 12 treatments (see below). Four weeks after sowing, six mycelium plugs of Trichoderma gamsii were added to each treatment tests, with the exception of the ‘negative control’.

1) Negative control. Soil without bentochar.

2) Positive control. Soil with addition of Trichodermagamsiuand without bentochar.

3) B-D1. Soil with bentochar (2%). No skopobiota added.

4) B-D2. Soil with bentochar (4%). No skopobiota added.

5) B-D3. Soil with bentochar (8%). No skopobiota added.

6) SK-1 D2.

7) SK-1 D3.

8) SK-2 D2.

9) SK-2 D3.

10) SK-3 D1.

11) SK-3 D2.

12) SK-3 D3.

Nine weeks after sowing, carrots were examined for three parameters: (1) above-ground growth, (2) number of fully unfolded leaves, and (3) presence of necrotic lesions or browning in roots. FIG. 31 shows carrots grown under different treatments, 9 weeks after sowing.

FIG. 32 shows root browning and root necrosis in carrots.

Table 6. shows growth parameters recorded in carrots grown under different treatments.

| | Above-ground growth |
|---|---|
| | (% of control) |
| | Mean ± SD |
| Negative control | 100% ± 31% |
| Positive control | 100% ± 20% |
| B-D treatments | 96% ± 20% |
| SK-1 D treatments | 106% ± 22% |
| SK-2 D treatments | 102% ± 24% |
| SK-3 D treatments | 111% ± 29% |

Effects of Biotic Stressors

A combination of factors, in which the presence of Trichoderma gamsii was determinant, induced the presence of root browning and necrotic lesion, as shown in FIG. 32.

Carrot roots of each biological replicate, grown under different treatments, were examined to assess differences in the development of root browning and necrotic lesions. Carrot roots that did not develop root browning or necrotic lesions were given a score of 0, roots that developed root browning were given a score of 1, while roots that developed 1 or more necrotic lesions (with or without the simultaneous presence of browning) were given a score of 2. The sum of the scores given to each biological replicate, per treatment, are summarized in the table below.

Table 7 shows the total score of root symptoms recorded in carrot roots grown under different treatments.

| | Total score |
|---|---|
| Negative control | 2 |
| Positive control | 10 |
| B-D treatments | 8 |
| SK-1 D treatments | 6 |
| SK-2 D treatments | 8 |
| SK-3 D treatments | 6 |

The presence of bentochar alone was sufficient to reduce the total score of root symptomatology, when compared with the positive control. However, the addition of skopobiota to the bentochar further reduced the total score. This shows a positive action of both bentochar and the included skopobiota in reducing the development of symptomatology caused by biotic factors.

Synergistic Effect of Skopobiota-Loaded Bentochar and Blad Spray

Similarly to what described in section ‘Effects on the growth of nine-week-old carrots’, the same skopobiota-loaded bentochar treatments were tested also in combination with a weekly spray of Blad on carrot leaves. The tested Blad concentration was 2 mL Blad per L of water.

In addition to the treatments described in ‘Effects on the growth of nine-week-old carrots’, there is:

13) Blad spray. Soil with addition of Trichodermagamsiiand without bentochar.

14) SK-1 D2 Blad.

15) SK-1 D3 Blad.

16) SK-2 D2 Blad.

17) SK-2 D3 Blad.

18) SK-3 D1 Blad.

19) SK-3 D2 Blad.

20) SK-3 D3 Blad.

The same parameters described in ‘Effects on the growth of nine-week-old carrots’ were assessed.

Biostimulant Effect of Blad, Blad+Bentochar & Blad+Bentochar+Skopobiota on Carrots Growth

The above-ground growth of carrots was positively affected by the weekly spray with Blad. However, the effect is evident in all treatments, but not in the Positive control. In fact, the above-ground growth of carrots in absence of skopobiota-loaded bentochar, with or without treatment with Blad was not observed for very young carrots. This shows that there is a synergistic effect of skopobiota-loaded bentochar and Blad, that results in an increased growth.

This is also evident in what concerns to the number of leaves, but in this case there was also an average increase in the number of leaves as induced by Blad (Positive control). In most treatments, as well as in the Positive control, the application of Blad increases the overall number of leaves, when compared with treatments without Blad.

Table 8 shows the growth parameters recorded in carrots grown under different treatments.

| | Above-ground growth | | Number of leaves | |
|---|---|---|---|---|
| | (% of control) | | (% of control) | |
| | Mean ± SD | | Mean ± SD | |
| | Without | With | Without | With |
| | Blad | Blad | Blad | Blad |
| Negative control | 100% ± 31% | — | 100% ± 11% | — |
| (no Blad added) | | | | |
| Positive control | 100% ± 20% | 98% ± | 96% ± 17%→ | 108% ± |
| (sprayed with | | 27% | | 17% |
| Blad) | | | | |
| B-D treatments | 96% ± 20% | — | 95% ± 23% | — |
| SK-1 D \| SK-1 | 106% ± 22%→ | 113% ± | 98% ± 19%→ | 115% ± |
| D Blad treatments | | 19% | | 17% |
| SK-2 D \| SK-2 | 102% ± 24%→ | 115% ± | 92% ± 19%→ | 98% ± |
| D Blad treatments | | 23% | | 20% |
| SK-3 D \| SK-3 | 111% ± 29%→ | 118% ± | 92% ± 10% | 92% ± |
| D Blad treatments | | 22% | | 25% |

Effects of Biotic Stressors

The spray with Blad strongly reduced the development of root symptoms when compared with the Positive control. A strong reduction is also observed when comparing most of the treatments, with and without the use of Blad. This suggests that the spray with Blad is capable reducing the development root browning and necrosis to near Negative control-level, under different skopobiota-loaded bentochar treatments.

Table 9 shows total score of root symptoms recorded in carrot roots grown under different treatments.

| | Total score | |
|---|---|---|
| | Without Blad | With Blad |
| Negative control | 2 | — |
| Positive control \| Blad Spray | 10 → | 2 |
| B-D treatments | 8 | — |
| SK-1 D3 \| SK-1 D3 Blad treatments | 6 | 6 |
| SK-2 D3 \| SK-2 D3 Blad treatments | 8 → | 2 |
| SK-3 D1 \| SK-3 D1 Blad treatments | 6 → | 2 |

### REFERENCES TO LITERATURE

Agrios G N (2005). Plant Pathology. 5th Edition. Amsterdam, the Netherlands: Elsevier Academic Press. ISBN: 9780120445653.
Amann R I, Ludwig W, Schleifer K H (1995) Phylogenetic identification and in situ detection of individual microbial cells without cultivation. Microbiological Reviews 59, 143-169.
Baldé A, Neves D, Garca-Breijo F J, Pais M S, Cravador A (2017) De novo assembly of Phlomis purpurea after challenging with Phytophthora cinnamomi BMC Genomics 18, 700. doi: 10.1186/s12864-017-4042-6
Baldi P, La Porta N (2017) Xylella fastidiosa. Host Range and Advance in Molecular Identification Techniques. Frontiers in Plant Science 8, 944. doi: 10.3389/fpls.2017.00944
Berg G, Grube M, Schloter M, Smalla K (2014) Unraveling the plant microbiome: looking back and future perspectives. Frontiers in Microbiology 5, 148. doi: 10.3389/fmicb.2014.00148
Berg G, Köberl M, Rybakova D, Muller H, Grosch R, Smalla F (2017) Plant microbial diversity is suggested as the key to future biocontrol and health trends. FEMS Microbiology Ecology 93, fix050. doi: 10.1093/femsec/fix050
Bever J D (1994) Feedback between plants and their soil communities in an old field community. Ecology 75, 1965-1977. doi: 10.2307/1941601
Blazewicz S J, Barnard R L, Daly R A, Firestone M K (2013) Evaluating rRNA as an indicator of microbial activity in environmental communities: limitations and uses. The International Society for Microbial Ecology Journal 7, 2061-2068. doi: 10.1038/ismej.2013.102
Brown, A V, Brasier C M (2007) Colonization of tree xylem by Phytophthora ramorum, P. kernoviae and other Phytophthora species. Plant Pathology 56, 227-241. doi: 10.1111/j.1365-3059.2006.01511.x
Bruez E, Baumgartner K, Bastien 5, Travadon R, Guérin-Dubrana L, Rey P (2016) Various fungal communities colonise the functional wood tissues of old grapevines externally free from grapevine trunk disease symptoms. Australian Journal of Grape and Wine Research 22, 288-295. doi: 10.1111/ajgw.12209
Bruez E, Vallance J, Gerbore J, Lecomte P, Da Costa J-P, Guerin-Dubrana L, Rey P (2014) Analyses of the temporal dynamics of fungal communities colonizing the healthy wood tissues of Esca leaf-symptomatic and asymptomatic vines. PLoS ONE 9(5), e95928. doi: 10.1371/journal.pone.0095928
Bulgarelli D, Rott M, Schlaeppi K, Ver Loren van Themaat E, Ahmadinejad N, et al (2012) Revealing structure and assembly cues for Arabidopsis root inhabiting bacterial microbiota. Nature 488, 91-95. doi: 10.1038/nature11336
Carabottia M, Scirocco A, Maselli M A, Severi C (2015) The gut-brain axis: interactions between enteric microbiota, central and enteric nervous systems. Annals of Gastroenterology 28, 203-209.
Carreira A, Ferreira J B, Pereira I, Ferreira J, Filipe P, Ferreira R B, Monteiro S (2018) Blad-Containing Oligomer: a novel fungicide used in crop protection as an alternative treatment for tinea pedis and tinea versicolor. Journal of Medical Microbiology 67,198-207. doi: 10.1099/jmm.0.000675
Casieri L, Hofstetter V, Viret O, Gindro K (2009) Fungal communities living in the wood of different cultivars of young Vitis vinifera plants. Phytopathologia Mediterranea 48, 73-83. doi: 10.14601/Phytopathol_Mediterr-2876
Coelho V (2009) Efeito do fosfonato de potbssio na proteceoo das raizes do castanheiro (Castanea sativa Mill.) contra Phytophthora cinnamomi M.Sc. thesis, Instituto Politécnico de Braganca, Braganca, Portugal. Available at: https://bibliotecadigital.ipb.pt/bitstream/10198/1200/1/Tese %20Mestrado %20%20%28 Valentim %20Coelho %29.pdf
Cristescu M E (2014) From barcoding single individuals to metabarcoding biological communities: towards an integrative approach to the study of global biodiversity. Trends in Ecology & Evolution 29, 566-571. doi: 10.1016/j.tree.2014.08.001
Dissanayake A, Purahong W, Wubet T, Hyde K D, Zhang W, Xu H, Zhang G, Fu C, Liu M, Xing Q, Li X, Yan J (2018) Direct comparison of culture-dependent and culture-independent molecular approaches reveal the diversity of fungal endophytic communities in stems of grapevine (Vitis vinifera). Fungal Diversity 90, 85-107. doi: 10.1007/s13225-018-0399-3
EFSA (2016) Scientific report on the update of a database of host plants of Xylella fastidiosa- 20 Nov. 2015. EFSA Journal 14, 4378-4418. doi: 10.2903/j.efsa.2016.4378
Erwin D C & Ribeiro O K (1996). Phytophthora Diseases Worldwide. Minnesota, USA: The American Phytopathological Society. ISBN: 0-89054-212-0.
Ferreira R B, Monteiro 5, Freitas R, Santos C N, Chen Z, Batista L M, Duarte J, Borges A & Teixeira A R (2006) Fungal pathogens: The battle for plant infection. Critical Reviews in Plant Sciences. 25, 505-524. doi: 10.1080/07352680601054610
Fitzpatrick C R, Copeland J, Wang P W, Guttman D S, Kotanen P M, Johnson M T J (2018) Assembly and ecological function of the root microbiome across angiosperm plant species. Proceedings of the National Academy of Sciences USA 115, (6) E1157-E1165. doi: 10.1073/pnas.1717617115
Friesen M L, Porter 55, Stark S C, Von Wettberg E J, Sachs J L, Martinez-Romero E (2011) Microbially mediated plant functional traits. Annual Review of Ecology, Evolution, and Systematics 42, 23-46. doi: 10.1146/annurev-ecolsys-102710-145039
Galland L (2014) The Gut Microbiome and the Brain. Journal of Medicinal Food 17, 1261-1272. doi: 10.1089/jmf.2014.7000
González V, Snchez-Torres P, Hinarejos R, Tuset J J (2009) Inonotus hispidus fruiting bodies on grapevines with esca symptoms in mediterranean areas of Spain. Journal of Plant Pathology 91, 465-468. doi: 10.4454/jpp.v91i2
Gonzalez V, Tello M L (2011) The endophytic mycota associated with Vitis vinifera in central Spain. Fungal Diversity 47, 29-42. doi: 10.1007/s13225-010-0073-x
Gramaje D, Urbez-Torres J R, Sosnowski M R (2018) Managing grapevine trunk diseases with respect to etiology and epidemiology current strategies and future prospects. Plant Disease 102, 12-39. doi: 10.1094/PDIS-04-17-0512-FE
Guest D, Grant B (1991) The complex action of phosphonates as antifungal agents. Biological Reviews 66, 159-187. doi: 10.1111/j.1469-185X.1991.tb01139.x
Hacquard S, Schadt C W (2014) Towards a holistic understanding of the beneficial interactions across the Populus microbiome. New Phytologist 205, 1424-1430. doi: 10.1111/nph.13133
Halleen F, Mostert L, Crous P W (2007) Pathogenicity testing of lesser-known vascular fungi of grapevines. Australasian Plant Pathology 36, 277-285. doi: 10.1071/AP07019
Haňáčková Z, Havrdová L, Čern\check{y} K, Zahradnik D, Koukol O (2017) Fungal endophytes in ash shoots—diversity and inhibition of Hymenoscyphus fraxineus. Baltic Forestry 23, 89-106.
Hardham A R, Blackman L M (2018). Pathogen profile update Phytophthora cinnamomi Molecular Plant Pathology 19, 260-285. doi: 10.1111/mpp.12568.
Haynes J D (2016) The developmental morphology of Anthostoma gastrinum. Mycologia, 61. 518-525. doi: 10.2307/3757241
Hugenholtz P, Goebel B M, Pace N R (1998) Impact of culture-independent studies on the emerging phylogenetic view of bacterial diversity. Journal of Bacteriology 180, 4765-4774.
Jaklitsch W M, Fournier J, Rogers J D, Voglmayr H (2014) Phylogenetic and taxonomic revision of Lopadostoma. Persoonia, 32, 52-82. doi: 10.3767/003158514X679272
Jayawardena R S, Purahong W, hang W, Wubet T, Li X, Liu M, Zhao W, Hyde K D, Liu J, Yan J (2018) Biodiversity of fungi on Vitis vinifera L. revealed by traditional and high-resolution culture-independent approaches. Fungal Diversity 90, 1-84. doi: 10.1007/s13225-018-0398-4
Kolařík M, Spakowicz D J, Gazis R, Shaw J, Kubátová A, Nováková A, Chudíčová M, Forcina G C, Kang K W, Kelnarová I, Skaltsas D, Portero C E, Strobel S A, Narváez-Trujillo A (2017) Biatriospora (Ascomycota: Pleosporales) is an ecologically diverse genus including facultative marine fungi and endophytes with biotechnological potential. Plant Systematics and Evolution, 303, 35-50. doi: 10.1007/s00606-016-1350-2
Landum M C, Felix M R, Alho J, Garcia R, Cabrita M J, Rei F, Varanda C M (2016) Antagonistic activity of fungi of Olea europaea L. against Colletotrichum acutatum. Microbiological Research 183, 100-108, doi: 10.1016/j.micres.2015.12.001
Larena I, Cal A, Melgarejo P (2004) Solid substrate production of Epicoccum nigrum conidia for biological control of brown rot on stone fruits. International Journ al of Food Microbiology 94, 161-167. doi: 10.1016/j.jfoodmicro.2004.01.007
Laroche O, Wood S A, Tremblay L A, Lear G, Ellis J I, Pochon X. (2017) Metabarcoding monitoring analysis: the pros and cons of using co-extracted environmental DNA and RNA data to assess offshore oil production impacts on benthic communities. PeerJ 5:e3347. doi: 10.7717/peerj.3347
Lebeis S L, Paredes S H, Lundberg D S, Breakfield N, Gehring J, McDonald M, Malfatti S, Glavina del Rio T, Jones C D, Tringe S G, Dangl J L (2015) Salicylic acid modulates colonization of the root microbiome by specific bacterial taxa. Science 349, 860-864. doi: 10.1126/science.aaa8764
Ling L L, Schneider T, Peoples A J, Spoering A L, Engels I, et al. (2015) A new antibiotic kills pathogens without detectable resistance. Nature 517, 455-459. doi: 10.1038/nature14098
Li Y, Xia L Q, Wang Y N, Liu X Y, Zhang C H, Hu T L, Cao K Q (2013) The inhibitory effect of Epicoccum nigrum strain XF1 against Phytophthora infestans. Biological Control 67, 462-468. doi: 10.1016/j.biocontrol.2013.09.007
Lundberg D S, Lebeis, S L, Paredes S H, Yourstone S, Gehring J, et al (2012) Defining the core Arabidopsis thaliana root microbiome. Nature 488, 86-90. doi: 10.1038/nature11237
Marco S, Osti O, Mugnai L (2011) First studies on the potential of a copper formulation for the control of leaf stripe disease within esca complex in grapevine. Phytopathologia Mediterrania 50 (Suppl), 5300-5309. doi: 10.14601/Phytopathol_Mediterr-10233
Mateus M C, Neves D, Dacunha B, Laczko E, Maia C, Teixeira R, Cravador A (2016) Structure, anti-Phytophthora and anti-tumor activities of a nortriterpenoid from the rhizome of Phlomis purpurea (Lamiaceae). Phytochemistry 131, 158-164. doi: 10.1016/j.phytochem.2016.09.004
Monteiro, S, Freitas, R, Rajasekhar B T, Teixeira A R, Ferreira R B (2010) The unique biosynthetic route from Lupinus β-conglutin gene to Blad. PLoS ONE 5(1), e8542. doi:10.1371/journal.pone.0008542
Monteiro S A, Carreira A, Freitas R, Pinheiro A M, Ferreira R B (2015) A nontoxic polypeptide oligomer with a fungicide potency under agricultural conditions which is equal or greater than that of their chemical counterparts. PLoS ONE 10(4), e0122095. doi:10.1371/journal.pone.0122095
Monteiro S A, Freitas R M, Teixeira A N, Ferreira R B (2003) Lupinus albus β-conglutin precursor mRNA. 1,791 nucleotides. Accession no. AY500372, GenBank.
Monteiro S A, Freitas R M, Teixeira A N, Ferreira R B (2006) Stable protein intermediary of Lupinus albus β-conglutin catabolism with defense properties. 519 nucleotides. Accession no. DQ142920, GenBank.
Morgan H H, Toit M, Setati M E (2017) The grapevine and wine microbiome: insights from high-throughput amplicon sequencing. Frontiers in Microbiology 8, 820. doi: 10.3389/fmicb.2017.00820
Musetti R, Polizzotto R, Vecchione A, Borselli S, Zulini L, D'Ambrosio M, Toppi L S, Pertot I (2007) Antifungal activity of diketopiperazines extracted from Alternaria alternata against Plasmopara viticola an ultrastructural study. Micron 38, 643-650. doi: 10.1016/j.micron.2006.09.001
Nelson S C (2008) Late Blight of Tomato (Phytophthora infestans). Plant Disease PD-45. https://www.ctahr.hawaii.edu/oc/freepubs/pdf/PD-45.pdf
Neves D, Caetano P, Oliveira J, Maia C., Horta M, Sousa N, Salgado M, Dionísio L, Magan N, Cravador A (2014) Anti-Phytophthora cinnamomi activity of Phlomis purpurea plant and root extracts. European Journal of Plant Pathology 138, 835-846. doi: 10.1007/s10658-013-0357-6
Nichols D, Cahoon N, Trakhtenberg E M, Pham L, Mehta A, Belanger A, Kanigan T, Lewis K, Epstein 55 (2010) Use of ichip for high-throughput in situ cultivation of “uncultivable” microbial species. Applied and Environmental Microbiology 76, 2445-2450. doi: 10.1128/AEM.01754-09
Nogales A, Nobre T, Valadas V, Ragonezi C, Doring M, Polidoros A, et al (2016) Can functional hologenomics aid tackling current challenges in plant breeding? Briefings in Functional Genomics 15, 288-297. doi: 10.1093/bfgp/elv030
Núnez-Trujillo G, Cabrera R, Burgos-Reyes R L, Silva E, Giménez C, Cosoveanu A (2012) Endophytic fungi from Vitis vinifera L. isolated in Canary Islands and Azores as potential biocontrol agents of Botrytis cinerea Pers.:Fr. Journal of Horticulture, Forestry and Biotechnology 16,1-6.
O'Niell J (2014) Review on antimicrobial resistance. Tackling drug-resistant infections globally. Wellcome Trust and the UK Government. URL: file:///E:/RBF-02Out2017-HD7/Info %20artigos,%201ivros %20et %20al/Microrganismos/MDR/Review %20on %20A MR_2014_Governo % 20UK.pdf
Ouimette D G, Coffey M D (1989) Comparative antifungal activity of four phosphonate compounds against isolates of nine Phytophthora species. Disease Control and Pest Management 79, 761-767. doi: 10.1094/Phyto-79-761
Pancher M, Ceol M, Comeo P E, Longa C M O, Yousaf S, Pertot I, Campisano A (2012) Fungal endophytic communities in grapevines (Vitis vinifera L.) respond to crop management. Applied and Environmental Microbiology 78, 4308-4317. doi: 10.1128/AEM.07655-11
Paulo S B M, Delvaux C J, Oliveira M N V, Moreira B C, Borges A C, Totola M R, Neves J C L, Costa M D (2017) Diversity and distribution of the endophytic fungal community in eucalyptus leaves. African Journal of Microbiology Research 11, 92-105. doi: 10.5897/AJMR2016.8353
Pawlowski J, Esling P, Lejzerowicz F, Cedhagen T, Wilding T A (2014) Environmental monitoring through protist NGS metabarcoding: assessing the impact of fish farming on benthic foraminifera communities. Molecular Ecology Resources 14, 1129-1140. doi: 10.1111/1755-0998.12261
Pinheiro A M, Carreira A, Rollo F, Fernandes R, Ferreira R B, Monteiro S (2016) Blad-containing oligomer fungicidal activity on human pathogenic yeasts. From the outside to the inside of the target cell. Frontiers in Microbiology 7, Art° 1803. doi: 10.3389/fmicb.2016.01803
Pinheiro A M, Carreira A, Prescott T, Ferreira R B, Monteiro S (2017) Bridging the gap to nontoxic fungal control: Lupinus-derived Blad Containing Oligomer as a novel candidate to combat human pathogenic fungi. Frontiers in Microbiology 8, 1182. doi: 10.3389/fmicb.2017.01182
Pinheiro A M, Carreira A, Ferreira R B, Monteiro S (2018) Fusion proteins towards fungi and bacteria in plant protection. Microbiology 164, 11-19. doi: 10.1099/mic.0.000592
Polizzotto R, D'Agostin S, Grisan S, Assante G, Pertot I, Andersen B (2009) Activity of endophytic Alternaria spp. strains in the control of Plasmopara viticola. Journal of Plant Pathology 91 (4, Supp), 54.79-80. doi: 10.4454/jpp.v91i4sup.581
Qin J, Li R, Raes J, Arumugam M, et al (2010) A human gut microbial gene catalogue established by metagenomic sequencing. Nature 464, 59-65. doi: 10.1038/nature08821
Ramos P C R, Ferreira R M S B, Franco E, Teixeira A R N (1997) Accumulation of a lectin-like breakdown product of β-conglutin catabolism in cotyledons of germinating Lupinus albus L. seeds. Planta, 203, 26-34. doi: 10.1007/s00050161
Roberti R, Osti F, Innocenti G, Rombolb A D, Di Marco S (2017) An environmentally sustainable approach for the management of Phaeoacremonium minimum, the main agent of wood diseases in Actinidia deliciosa. European Journal of Plant Pathology 148, 151-162. doi: 10.1007/s10658-016-1079-3
Santos C A, Sobrinho N M B A, Costa E S P, Diniz C S, Carmo M G F (2017) Liming and biofungicide for the control of clubroot in cauliflower. Pesquisa Agropecu\acute{a}ria Tropical Goi\hat{a}nia 47, 303-311. doi: 10.1590/1983-40632016v4746936
Santos-Medellin C, Edwards J, Liechty Z, Nguyen B, Sundaresan V (2017) Drought stress results in a compartment-specific restructuring of the rice root-associated microbiomes. mBio 8:e00764-17. doi: 10.1128/mBio.00764-17
Staley J T, Konopka A (1985) Measurement of in situ activities of nonphotosynthetic microorganisms in aquatic and terrestrial habitats. Annual Review of Microbiology 39, 321-346. doi: 10.1146/annurev.micro.39.1.321
Suetrong S, Hyde K D, Zhang Y, Bahkali A H, Jones G (2011) Trematosphaeriaceae fam. nov. (Dothideomycetes, Ascomycota). Cryptogamie, Mycologie 32, 343-358. doi: 10.7872/crym.v32.iss4.2011.343
Surico G (2009) Towards a redefinition of the diseases within the esca complex of grapevine. Phytopathologia Mediterranea 48, 5-10. doi: 10.14601/Phytopathol_Mediterr-2870
Thambugala K M, Hyde K D, Tanaka K, Tian Q, Wanasinghe D N, Ariyawansa H A, Liu Z Y (2015) Towards a natural classification and backbone tree for Lophiostomataceae, Floricolaceae, and Amorosiaceae fam. nov. Fungal Diversity 74, 199-266. doi: 10.1007/s13225-015-0348-3
Thines M (2013). Taxonomy and Phylogeny of Phytophthora and Related Oomycetes. In: Lamour K (Ed). Phytophthora: A Global Perspective. Wallingford, UK: CABI.
Van Der Heijden M G A, Schlaeppi K (2015) Root surface as a frontier for plant microbiome research. Proc Natl Acad Sci USA 112, 2299-2300. doi: 10.1073/pnas.1500709112
Varanda C M R, Oliveira M, Materatski P, Landum M, Clara M I E, Felix M R (2016) Fungal endophytic communities associated to the phyllosphere of grapevine cultivars under different types of management. Fungal Biology 120, 1525-1536. doi: 10.1016/j.funbio.2016.08.002
Vicente M R-S, Plasencia J (2011) Salicylic acid beyond defence: its role in plant growth and development. Journal of Experimental Botany 62, 3321-3338. doi: 10.1093/jxb/err031
Wikipedia contributors (2018, December 27). Fecal microbiota transplant. In Wikipedia, The Free Encyclopedia. Retrieved 12:02, Feb. 17, 2019, from https://en.wikipedia.org/w/index.php?title=Fecal_microbiota_transplant&oldid=87 5511741
Xing X K, Chen J, Xu M J, Lin W H, Guo S X (2011) Fungal endophytes associated with Sonneratia (Sonneratiaceae) mangrove plants on the south coast of China. Forest Pathology 42 334-340. doi: 10.1111/j.1439-0329.2010.00683.x

1. Use of an adaptogen for the treatment and/or control and/or prevention of a disease or infestation of a plant by application of said adaptogen to the plant, wherein:
(1) the adaptogen is Blad or a variant thereof and the location of the disease or infestation is in a region of the plant which is different to the region where the adaptogen is applied; or
(ii) the adaptogen is skopobiota which is used to inoculate said plant or part thereof;
and preferably said adaptogen is in the form of a composition.

2. Use according to claim 1 of a composition for the treatment and/or control and/or prevention of a disease or infestation of a plant by application of said composition to the plant, wherein the location of the disease or infestation is in a region of the plant which is different to the region where the composition is applied, wherein said composition comprises a polypeptide comprising the Blad sequence shown in SEQ ID NO: 4 or an active variant thereof which has Blad activity and which comprises a sequence which has at least 70% identity to either SEQ ID NO: 4 or a fragment of SEQ ID NO: 4 which is at least 100 amino acids in length.

3. Use according to claim 1 or 2, for the treatment and/or control and/or prevention of a disease or infestation of the vasculature of a plant by application of said composition to non-vasculature regions of a plant; wherein said composition has an antimicrobial agent which comprises a polypeptide comprising the Blad sequence shown in SEQ ID NO: 4 or an active variant thereof which has antimicrobial activity and which comprises a sequence which has at least 70% identity to either SEQ ID NO: 4 or a fragment of SEQ ID NO:4 which is at least 100 amino acids in length.

4. Use according to claim 1 of a composition comprising skopobiota for the treatment and/or control and/or prevention of a disease or infestation of a plant by inoculation of said plant or part thereof with said composition.

5. Use according to claim 4 for the treatment and/or control and/or prevention of a disease or infestation of the vasculature of a plant by inoculation of said plant or part thereof with said composition.

6. Use according to claim 4 or 5, wherein said skopobiota comprises fungi selected from endophytes and/or saprophytes of healthy plant vasculatures, optionally in combination with one or more pathogenic fungi.

7. Use according to claim 6, wherein said skopobiota comprises two or more endophytes or saprophytes selected from the following genera: Absidia Acremonium Acrocalymma Acrospermum Acrostalagmus Actinomucor Agaricus Albifimbria Alfaria Alternaria Amerosporium Ampelomyces Amphisphaeria Angustimassarina Annulohypoxylon Anthostoma Apiotrichum Aplosporella Apodus Arachnomyces Armillaria Arthrinium Arthrobotrys Arthrographis Articulospora Arxiomyces Ascobolus Ascochyta Ascorhizoctonia Aspergillus Asperisporium Athelia Aureobasidium Bactrodesmium Bartalinia Beauveria Bensingtonia Bertia Biatriospora Bionectria Bipolaris Biscogniauxia Bjerkandera Blumena Boeremia Botryodiplodia Botryosphaeria Botrytis Briosia Buckleyzyma Cadophora Calonectria Caloplaca Calycella Camarosporium Camillea Campylocarpon Candida Capnodium Capronia Catenulostroma Cenococcum Cephalosporium Ceratobasidium Cercospora Chaetomium Chaetothyrium Chalastospora Cheilymenia Chrysosporium Circinotrichum Citeromyces Cladochytrium Cladophialophora Cladosporium Clathrospora Clathrus Claviceps Clitopilus Clonostachys Cochliobolus Colacogloea Colletotrichum Collophorina Coniella Coniocessia Coniochaeta Coniolariella Coniothecium Coniothyrium Cophinforma Coprinellus Coriolopsis Corticium Coryneopsis Corynespora Coryneum Crepidotus Cryptocine Cryptococcus Cryptophaeella Cryptosphaeria Cryptosporella Cryptovalsa Curvularia Cutaneotrichosporon Cyberlindnera Cylindrocarpon Cylindrodadiella Cyphellophora Cystobasidium Cystofilobasidium Cytospora Dacrymyces Dactylellina Dactylonectria Debaryomyces Deconica Dendrophoma Desarmillaria Desmazierella Devriesia Diaporthe Diatrype Diatrypella Dictyosporium Didymella Didymosphaeria Dinemasporium Dioszegia Diplodia Diplodina Discohainesia Discosia Discostroma Doratomyces Dothiorella Elsinoe Emericella Endobasidium Endoconidioma Engyodontium Epicoccum Eriocercosporella Eriosphaeria Erysiphe Erythricium Erythrobasidium Eucasphaeria Eutypa Eutypella Excipula Exidia Exobasidium Exophiala Exosporium Exserohilum Fellomyces Filobasidium Floricola Fomes Fomitiporella Fomitiporia Funneliformis Fusarium Fuscoporia Fusicladium Ganoderma Geomyces Geotrichum Gibellulopsis Glomerella Gloniopsis Glonium Golovinomyces Gonatobotrys Gonatobotryum Graphium Greeneria Grovesinia Guehomyces Gymnascella Gymnopus Gyrothrix Hannaella Hanseniaspora Hansfordia Hapalopilus Helicobasidium Helminthosporium Hendersonia Herpotrichia Heterobasidion Holtermanniella Hormonema Humicola Hyaloceras Hydnum Hymenochaetopsis Hyphoderma Hyphodermella Hyphodontia Hypocrella Hypoderma Hypoxylon Hysterium Hysterobrevium Hysterographium Ilyonectria Inocutis Inonotus IrpexItersonilia Kalmusia Karstenula Kazachstania Kernia Kluyveromyces Knufia Kondoa Kuehneola Kurtzmanomyces Lachancea Lachnella Lachnum Laetiporus Lasiodiplodia Lecanicillium Lecanidion Lecythophora Lentinus L enzites Leprocaulon Leptodothiorella Leptosphaeria Leptothyrium Leucosporidium Leucostoma Libertella Lopadostoma Lophidium Lophiostoma Lophiotrema L oranitschkia Lycoperdon Macrophoma Macrophomina Macrosporium Magnaporthe Malassezia Marasmius Mariannaea Marssonina Massariella Massarina Meira Meliola Merismodes Metarhizium Metasphaeria Metschnikowia Meyerozyma Microascus Microdiplodia Microdochium Micropera Microthyrium Minimedusa Moeszia Mollisia Monilinia Monochaetia Monochaetinula Monodictys Monographella Mortierella Mrakia Mucor Mycena Mycosphaerella Myrothecium Myxosporium Naganishia Nectria Nemania Neoanthostomella Neodevriesia Neoerysiphe Neofusicoccum Neomassaria Neonectria Neopestalotiopsis Neophysalospora Neoplaconema Neoscytalidium Neurospora Nrospora Nodulisporium Occultifur Oidiodendron Ophiocordyceps Ophiostoma Orbilia Ostreichnion Ostreola Paecilomyces Papiliotrema Papulospora Paraconiothyrium Paraphaeosphaeria Paraphoma Parasola Pareutypella Passalora Patellaria Penicillium Peniophora Peniophorella Penzigomyces Perenniporia Periconia Pestalotiopsis Petriella Phacidiella Phaeoacremonium Phaeococcomyces Phaeomoniella Phaeotheca Phaeotrichoconis Phakopsora Phallus Phanerochaete Phelinidium Phellinus Phialemoniopsis Phialophora Phialosimplex Phlebia Phlebiopsis Phoma Phyllosticta Physcia Phytophthora Pilidium Pionnotes Plagiostoma Plasmopara Pleospora Pleurophoma Pleurostoma Pleurotus Podospora Preussia Psathyrella Pseudallescheria Pseudocamarosporium Pseudocercospora Pseudogymnoascus Pseudolachnea Pseudopestalotiopsis Pseudopezicula Pseudotaeniolina Pseudozyma Psiloglonium Punctulariopsis Pyrenochaeta Pyrenophora Pyrgemmula Pythium Ramicandelaber Ramichloridium Ramularia Rhabdospora Rhinocladiella Rhizoctonia Rhizomucor Rhizopus Rhodosporidium Rhodotorula Robillarda Roesleria Rosellinia Saccharomyces Sakaguchia Sarcoporia Sarocladium Scheffersomyces Schizophyllum Schizothyrium Sclerostagonospora Sclerotinia Sderotium Scolicotrichum Scopulariopsis Scopuloides Scytalidium Scytinostroma Seimatosporium Seiridium Selenophoma Septoria Septoriella Setophaeosphaeria Simplicillium Sistotremastrum Solicoccozyma Sordaria Spencermartinsia Sphaeropsis Spiromastix Spiromyces Sporidiobolus Sporobolomyces Sporocadus Sporoschisma Stachybotrys Stagonospora Stemphylium Stereum Stergmatomyces Stigmina Strickeria Stromatoneurospora Talaromyces Taphrina Terana Tetracladium Tetracoccosporium Thanatephorus Thaxteriella Thelonectria Thielavia Tilletiopsis Tomentella Torula Torulaspora Toxicocladosporium Trametes Trematosphaeria Trichaptum Trichocladium Trichoderma Trichosporon Trichothecium Trullula Truncatella Tubaria Typhula Ulocladium Umbelopsis Uncispora Valsaria Veronaea Verpa Verrucocladosporium Verticillium Vishniacozyma Volutella Wallemia Xanthoria Xeromyces Xerotus Xylaria Xylodon Yamadazyma Zetiasplozna Zymoseptoria.

8. A use according to claim 5, wherein said skopobiota comprises fungi selected from at least 3, at least 4, or at least 5 genera.

9. A use according to claim 7, wherein said skopobiota comprises one or more of the following fungi: Alternaria, alternata, Epicoccum nigrum, Cladosporium sp., Aureolasidium pullulans.

10. A method of treatment and/or control and/or prevention of a disease or infestation of a plant by inoculation of said plant or part thereof with a composition containing skopobiota or at least one of said group of endophytes or saprophytes and by the external application of a composition containing an antimicrobial agent comprising a polypeptide comprising the Blad sequence shown in SEQ ID NO: 4 or an active variant thereof which has antimicrobial activity and which comprises a sequence which has at least 70% identity to either SEQ ID NO:4 or a fragment of SEQ ID NO: 4 which is at least 100 amino acids in length.

11. A method according to claim 10, wherein said skopobiota comprises fungi selected from endophytes and/or saprophytes of healthy plant vasculatures; optionally in combination with pathogenic fungi.

12. A method according to either claim 10 or claim 11, wherein said Skopobiota comprises two or more endophytes or saprophytes selected from the following genera: Absidia Acremonium Acrocalymma Acrospermum Acrostalagmus Actinomucor Agaricus Albifimbria Alfaria Alternaria Amerosporium Ampelomyces Amphisphaeria Angustimassarina Annulohypoxylon Anthostoma Apiotrichum Aplosporella Apodus Arachnomyces Armillaria Arthrinium Arthrobotrys Arthrographis Articulospora Arxiomyces Ascobolus Ascochyta Ascorhizoctonia Aspergillus Asperisporium Athelia Aureobasidium Bactrodesmium Bartalinia Beauveria Bensingtonia Bertia Biatriospora Bionectria Bipolaris Biscogniauxia Bjerkandera Blumeria Boeremia Botryodiplodia Botryosphaeria Botrytis Briosia Buckleyzyma Cadophora Calonectria Caloplaca Calycella Camarosporium Camillea Campylocarpon Candida Capnodium Capronia Catenulostroma Cenococcum Cephalosporium Ceratobasidium Cercospora Chaetomium Chaetothyrium Chalastospora Cheilymenia Chrysosporium Circinotrichum Citeromyces Cladochytrium Cladophialophora Cladosporium Clathrospora Clathrus Claviceps Clitopilus Clonostachys Cochliobolus Colacogloea Colletotrichum Collophorina Coniella Coniocessia Coniochaeta Coniolariella Coniothecium Coniothyrium Cophinforma Coprinellus Coriolopsis Corticium Coryneopsis Corynespora Coryneum Crepidotus Cryptocline Cryptococcus Cryptophaeella Cryptosphaeria Cryptosporella Cryptovalsa Curvularia Cutaneotrichosporon Cyberlindnera Cylindrocarpon Cylindrocladiella Cyphellophora Cystobasidium Cystoflobasidium Cytospora Dacrymyces Dactylellina Dactylonectria Debaryomyces Deconica Dendrophoma Desarmillaria Desmazierella Devriesia Diaporthe Diatrype Diatrypella Dictyosporium Didymella Didymosphaeria Dinemasporium Dioszegia Diplodia Diplodina Discohainesia Discosia Discostroma Doratomyces Dothiorella Elsinoe Emericella Endobasidium Endoconidioma Engyodontium Epicoccum Eriocercosporella Eriosphaeria Erysiphe Erythricium Erythrobasidium Eucasphaeria Eutypa Eutypella Excipula Exidia Exobasidium Exophiala Exosporium Exserohilum Fellomyces Filobasidium Floricola Fomes Fomitiporella Fomitiporia Funneliformis Fusarium Fuscoporia Fusicladium Ganoderma Geomyces Geotrichum Gibellulopsis Glomerella Gloniopsis Glonium Golovinomyces Gonatobotrys Gonatobotryum Graphium Greeneria Grovesinia Guehomyces Gymnascella Gymnopus Gyrothrix Hannaella Hanseniaspora Hansfordia Hapalopilus Helicobasidium Helminthosporium Hendersonia Herpotrichia Heterobasidion Holtermanniella Hormonema Humicola Hyaloceras Hydnum Hymenochaetopsis Hyphoderma Hyphodermella Hyphodontia Hypocrella Hypoderma Hypoxylon Hysterium Hysterobrevium Hysterographium Ilyonectria Inocutis Inonotus Irpex ftersonilia Kalmusia Karstenula Kazachstania Kernia Kluyveromyces Knufia Kondoa Kuehneola Kurtzmanomyces Lachancea Lachnella Lachnum Laetiporus Lasiodiplodia Lecanicillium Lecanidion Lecythophora Lentinus Lenzites Leprocaulon Leptodothiorella Leptosphaeria Leptothyrium Leucosporidium Leucostoma Libertella Lopadostoma Lophidium Lophiostoma Lophiotrema L oranitschkia Lycoperdon Macrophoma Macrophomina Macrosporium Magnaporthe Malassezia Marasmius Mariannaea Marssonina Massariella Massarina Meira Mebiola Merismodes Metarhizium Metasphaeria Metschnikowia Meyerozyma Microascus Microdiplodia Microdochium Micropera Microthyrium Minimedusa Moeszia Mollisia Monilinia Monochaetia Monochaetinula Monodictys Monographella Mortierella Mrakia Mucor Mycena Mycosphaerella Myrothecium Myxosporium Naganishia Nectria Nemania Neoanthostomella Neodevriesia Neoerysiphe Neofusicoccum Neomassaria Neonectria Neopestalotiopsis Neophysalospora Neoplaconema Neoscytalidium Neurospora Nigrospora Nodulisporium Occultifur Oidiodendron Ophiocordyceps Ophiostoma Orbilia Ostreichnion Ostreola Paecilomyces Papiliotrema Papulospora Paraconiothyrium Paraphaeosphaeria Paraphoma Parasola Pareutypella Passalora Patellaria Penicillium Peniophora Peniophorella Penzigomyces Perenniporia Periconia Pestalotiopsis Petriella Phacidiella Phaeoacremonium Phaeococcomyces Phaeomoniella Phaeotheca Phaeotrichoconis Phakopsora Phallus Phanerochaete Phelinidium Phellinus Phialemoniopsis Phialophora Phialosimplex Phlebia Phlebiopsis Phoma Phyllosticta Physcia Phytophthora Pilidium Pionnotes Plagiostoma Plasmopara Pleospora Pleurophoma Pleurostoma Pleurotus Podospora Preussia Psathyrella Pseudallescheria Pseudocamarosporium Pseudocercospora Pseudogymnoascus Pseudolachnea Pseudopestalotiopsis Pseudopezicula Pseudotaeniolina Pseudozyma Psiloglonium Punctulariopsis Pyrenochaeta Pyrenophora Pyrgemmula Pythium Ramicandelaber Ramichloridium Ramularia Rhabdospora Rhinocladiella Rhizoctonia Rhizomucor Rhizopus Rhodosporidium Rhodotorula Robillarda Roesleria Rosellinia Saccharomyces Sakaguchia Sarcoporia Sarocladium Scheffersomyces Schizophyllum Schizothyrium Sclerostagonospora Sclerotinia Sderotium Scolicotrichum Scopulariopsis Scopuloides Scytalidium Scytinostroma Seimatosporium Seiridium Selenophoma Septoria Septoriella Setophaeosphaeria Simplicillium Sistotremastrum Solicoccozyma Sordaria Spencermartinsia Sphaeropsis Spiromastix Spiromyces Sporidiobolus Sporobolomyces Sporocadus Sporoschisma Stachybotrys Stagonospora Stemphylium Stereum Sterigmatomyces Stigmina Strickeria Stromatoneurospora Talaromyces Taphrina Terana Tetracladium Tetracoccosporium Thanatephorus Thaxteriella Thelonectria Thielavia Tilletiopsis Tomentella Torula Torulaspora Toxicocladosporium Trametes Trematosphaeria Trichaptum Trichocladium Trichoderma Trichosporon Trichothecium Trullula Truncatella Tubaria Typhula Ulocladium Umbelopsis Uncispora Valsaria Veronaea Verpa Verrucocladosporium Verticillium Vishniacozyma Volutella Wallemia Xanthoria Xeromyces Xerotus Xylaria Xylodon Yamadazyma Zetiasplozna Zymoseptoria.

13. A method according to claim 12, wherein said skopobiota comprises:
fungi selected from at least 3, at least 4, or at least 5 genera, and/or
a microorganism selected from any list, figure or table disclosed herein, and/or
at least 5, 10 or 20 species of microorganisms from any list, figure or table disclosed herein.

14. A method according to either claim 12 or claim 13, wherein said skopobiota comprises one or more of the following fungi: Alternaria alternata, Epicoccum nigrum, Cladosporium sp., Aureolasidium pullulans.

15. A method according to any one of claims 10 to 14, wherein said Blad or variant containing composition is applied to foliage.

16. Use or method according to any one of the preceding claims, wherein:
said disease relates to the xylem of grapevines or any grapevine trunk disease, and is optionally esca-related, and/or
said adaptogen is applied to a plant part that is at least 0.1 m, at least 0.2 m, at least 0.3 m, at least 0.4 m, or at least 0.5 m away from the location of the disease in the plant, and/or
said skopobiota comprises at least 10 different species of microorganism which are present in a healthy microorganism population in or on the plant, and/or
said skopobiota comprises at least 5 species of microorganism which are not present in any microorganism population which is present in or on the plant, and/or
the microorganism populations present in or on the plant have been affected by an environmental factor or a disease, and/or
inoculating the plant with skopobiota increases the amount of at least 5 species of microorganisms present in a population in or on the plant by at least 50%.

17. Use or method according to claim 16, wherein said esca-related disease is one or more of the following: Phaeomoniella chlamydospora, Phaeoacremonium minimum and Fornitiporia mediterranea.

18. Use or method according to any of the preceding claims, wherein said plant is a grapevine.

19. Use or method according to any one of claims 1 to 15, wherein said disease is related to the genus Phytophthora.

20. Use or method according to any one of the preceding claims, wherein said plant is a tree, preferably oak or a chestnut tree.

21. Use or method according to any one of the preceding claims, wherein said plant is an herbaceous plant.

22. Use or method according to any of the preceding claims, wherein said disease is bacterial.

23. Use or method according to claim 22, wherein said bacterial disease includes Xylella fastidiosa.

24. Use or method according to claim 23, wherein said plant is an olive tree.

25. Use or method according to any one of the preceding claims, wherein said plant is a kiwi plant.

26. Use or method according to any one of the preceding claims, wherein said disease is cauliflower clubroot.

27. Use or method according to any of the preceding claims, wherein said plant is cauliflower.

28. Use or method according to any one of the preceding claims wherein:
said plant is typed or diagnosed in some way to identify the type and/or location of disease; and/or
the skopobiota is a characterized defined population of microorganisms, which optionally has been chosen based on typing or diagnosis of the plant; and/or
the plant is typed or diagnosed after the adaptogen is applied to monitor the activity of the adaptogen against the disease.

29. A method of detection of a disease in the vasculature of an externally asymptomatic plant comprising the steps of inoculation of said plant or part thereof with a composition containing skopobiota, the external application of a composition containing an antimicrobial agent that comprises a polypeptide comprising the Blad sequence shown in SEQ ID NO:4 or an active variant thereof which has antimicrobial activity and which comprises a sequence which has at least 70% identity to either SEQ ID NO:4 or a fragment of SEQ ID NO:4 which is at least 100 amino acids in length; and obtaining an image of part of the vasculature of said plant, preferably by X-ray or tomography, in order to determine the extent of any infection present therein.

30. Method of assessing the health of a plant comprising typing the microbiome of the plant to thereby determine whether the plant is in need of inoculation with microorganisms to improve its microbiome, wherein the typing preferably comprises DNA barcoding of the microbiome, preferably by typing or sequencing of any region of the genome defined herein.

31. Use of a skopbiota for the treatment and/or control and/or prevention of a disease or infestation of a plant by application of said skopobiota to soil where the plant is growing, wherein a porous material comprising biochar or a mixture of bentonite and biochar is contacted with the skopbiota to enhance delivery of the skopobiota to the plant.

32. A method of treatment and/or control and/or prevention of a disease or infestation of a plant by inoculation of said plant or part thereof with a skopobiota, wherein said skopbiota is contacted with soil in which the plant is growing and its delivery to the plant is enhanced by use of a porous material comprising biochar or a mixture of bentonite and biochar.

33. A use or method according to claim 31 or 32 wherein:
the skopobiota and/or plant is as defined in any one of the preceding claims; and/or
the disease or infestation is as defined in any one of the preceding claims; and/or
said use or method is combined with any use or method according to the any one of the preceding claims.

34. A method or use according to any one of claims 31 to 33 wherein:
the porous material comprises at least 30% or 50% of biochar by weight; and/or
the porous material comprises at least 30% or 50% of a mixture of biochar and bentonite by weight; and/or
the porous material comprises bentochar, for example at least 30% or 50% bentochar by weight; and/or
the skopobiota is contacted with the soil in the form of a mixture with the porous material; and/or
the porous material releases the skopobiota into soil over an extended period of time, for example over at least 3, 5, 10 or 20 days.

35. Bentochar, preferably in association with a skopbiota as defined in any one of the preceding claims.
